(19) United States
(12) Patent Application Publication
(10) Pub. No.: US 2021/0340550 A1
(43) Pub. Date: Nov. 4, 2021

(54) NUCLEIC ACID MOLECULES INSERTED EXPRESSION REGULATION SEQUENCES, EXPRESSION VECTOR COMPRISING NUCLEIC ACID MOLECLUES AND PHARMACEUTICAL USE THEREOF

(71) Applicant: THE CATHOLIC UNIVERSITY OF KOREA INDUSTRY-ACADEMIC COOPERATION FOUNDATION, Seoul (KR)

(72) Inventors: Jae Hwan NAM, Goyang-si (KR); Hyo Jung PARK, Seoul (KR); Hae Li KO, Bucheon-si (KR); Hun KIM, Suwon-si (KR); Hae Won KWAK, Seoul (KR)

(73) Assignee: THE CATHOLIC UNIVERSITY OF KOREA INDUSTRY-ACADEMIC COOPERATION FOUNDATION, Seoul (KR)

(21) Appl. No.: 17/050,503

(22) PCT Filed: Apr. 26, 2019
(86) PCT No.: PCT/KR2019/005099

§ 371 Date: Oct. 26, 2020

(30) Foreign Application Priority Data
Apr. 27, 2018 (KR) 10-2018-0049272

(51) Int. Cl. C12N 15/67; C12N 7/00

(52) U.S. Cl. CPC C12N 15/67; C12N 7/00; C12N 2840/203; C12N 2840/60; C12N 2770/24021; C12N 2770/32021; C12N 2770/36021; C12N 2770/22022; C12N 2840/105

(57) ABSTRACT

A nucleic acid molecule including at least one expression control sequence having an Internal Ribosomal Entry Site (IRES) sequence, at least one coding region, and optionally multiple adenosines or thymidines upstream of the at least one expression control sequence is disclosed as an expression system. Besides, a recombinant expression vector including the nucleic acid molecule and pharmaceutical or medicinal use of the nucleic acid molecule are disclosed.

### REFERENCE TO SEQUENCE LISTING SUBMITTED VIA EFS-WEB

This application includes an electronically submitted sequence listing in .txt format. The .txt file contains a sequence listing entitled “2021-05-06_6245-0117PUS1_ST25.txt” created on May 6, 2021 and is 46,193 bytes in size. The sequence listing contained in this .txt file is part of the specification and is hereby incorporated by reference herein in its entirety.

### TECHNICAL FIELD

The present disclosure relates to a nucleic acid molecule, and more specifically, to a nucleic acid molecule enhancing expression efficiency, an expression vector comprising the nucleic acid molecule and pharmaceutical use thereof.

### BACKGROUND ART

As biotechnology has been developed, various expression systems that express a gene of Interest (GOI) have been known. Among the expression systems, cell-based expression systems typically uses natural expression mechanisms of micro-organisms or eukaryotes, while other expression systems generally use purified RNA polymerases, ribosome, tRNA and ribonucleotides. In particular, proteins originated from eukaryotes perform post-translational modification such as phosphorylation, methylation and glycosylation. Since micro-organisms do not have such post-translational modification mechanisms, eukaryotic expression systems have been used in case expressing eukaryotic originated proteins.

Eukaryotic expression systems may be utilized to a gene therapy in which GOI having an open reading frame (ORF) encoding a peptide or a protein for curing various diseases is inserted in the expression systems or to a genetic vaccine in which GOI having ORF encoding a peptide or a protein such as antigens is inserted in the expression systems. The expression systems generally use nucleic acid sequences regulating transcription and/or translation of GOI so that they can express GOI efficiently within thereof. Typically, the expression systems enhance transcriptional efficiency using promoters with enhanced transcriptional efficiency, and use capping system unique to eukaryotes with regard to improving translation efficiency of GOI.

Capping system typically haw 5′ cap structure of 7-methyl-guanosine (m7G) at 5′ end so as to translate GOI efficiently. Translation Initiation Complex comprising translational regulation factors of eukaryotes such as eI4FA, eIF4E and eIF4G recognizes and binds to 5′ cap site to form capping structure and to initiate translation for synthesizing proteins. When the capping structure is formed at the translation initiation site, the capping structure initiates protein synthesis, while it prevents mRNA degradations by nuclease actions.

It is necessary to perform in vitro transcription (IVT) process to fabricate a nucleic acid molecule with the capping structure. For example, the nucleic acid molecule with the capping structure may be fabricated by treating plasmid DNA (pDNA) with restriction enzymes so as to linearize the pDNA, translating the linearized pDNA using RNA polymerases to fabricate mRNA, and attaching m7G(5′)-ppp-(5′)G, i.e. regular cap analog to the mRNA at 5′ end to make capped mRNA. However, such a cap analog often binds to 5′ end with opposite direction, and m7G nucleotides cannot act as a cap. About one of third among the fabricated mRNA does not have methylation at the cap site, and such mRNA cannot initiate protein synthesis.

Alternatively, IVT process was performed without the cap analog, and then, cap reaction was performed using commercially available vaccinia virus capping enzymes. Besides, protein synthesis can be induced using anti-reverse cap analog (ARCA) which prevents the reverse direction reaction of the cap (ARCA-capped mRNA). It has been known that ARCA-capped mRNA can synthesize proteins as twice as the regular cap analog-capped mRNA and has much longer half-life. However, performing an artificial capping reaction (e.g. ARCA reaction) in vitro is very expensive and has low efficiency. Accordingly, it is necessary to develop an expression system that has increasing efficiencies and can be utilized as a genetic vaccine, a gene therapy, and the likes.

Immune system means a biological structure or a mechanism that detects and removes pathogens or cancer cells within an organism and thereby, protecting the organisms from various diseases. The immune system may be divided into innate immune system (inherent immune system, natural immune system) and adaptive immune system (acquired immune system).

The innate immune system is mechanism that defends a host so as to avoid an infection un-specifically and instantly responds to the pathogens without memorizing a specific pathogen. All kinds of animals and plants have innate immune system, and plants, fungi and insect have only innate immune system. In contrast, the adaptive immune system is specific to an antigen or a pathogen, and it is necessary to recognize non-self antigen through antigen-presentation process in the adaptive immune system. Accordingly, it is possible to induce a specific immune response against a specific antigen or against cells infected by the specific antigens through the adaptive immune system. Since memory cells of the adaptive immune system can recruit immune response that was performed in past, it is possible to remove the pathogen rapidly when the same pathogen infiltrate to body.

In addition, immune system can be divided into humoral immunity and cell-mediated immunity (CMI). In the humoral immunity, B lymphocyte derived from a bone-marrow recognizes antigens, differentiates to secrete antibodies consisting of glycol-protein, i.e. immunoglobulin (Ig), and then the secreted antibodies remove the infected pathogens. The CMI is an immune response that T lymphocytes derived thymus recognizes antigens so as to secrete lymphokines or kill the infected cells directly.

Vaccine antigens, which inoculate a whole pathogen or a part thereof for inducing immune responses against to the pathogens, have been used for preventions or treatments of various diseases. In this case, it is preferable to induce various immune responses caused by the vaccine antigens. Recently, sub-unit vaccines have been mainly developed in placed of early-developed attenuated live vaccines or inactivated killed vaccines because the sub-unit vaccines contain evident structures and components. However, the sub-unit vaccines use adjuvant for enhancing immune responses since the vaccines show lower immunogenicity compared to the prior art vaccines.

Since antibodies act as primary defense actors against most of pathogenic bacteria or viruses, only antibodies induced by vaccine antigens can prevent various diseases. But, since cell-mediated immune responses act significantly on infection diseases against which vaccines have not been developed in preventions or treatments. In this case, it is possible to develop vaccines efficiently when using adjuvant inducing cell-mediated immune response.

Currently, alum, metal salts such as aluminum hydroxide, aluminum phosphate or aluminum hydroxide phosphate sulphate, and MF59, oil-in-water emulsion type adjuvant based on squalene, have been mainly used as adjuvants for human vaccines adjuvant. Such commonly used adjuvants induce little cell-mediated immunity while induce mainly humoral immunity. Accordingly, such adjuvants can be utilized only in case antibodies can defend infections, and they were not proper for vaccines requiring cell-mediated immune responses.

Micro-organisms as the typical pathogens have pathogen-associated molecular patterns (PAMPs) such as lipopolysaccharide (LPS), betha-1,3-glucan and peptidoglycans in cell walls thereof. A specific protein consisting of immune system of a host, for example, pattern recognition receptors (PRRs) or pattern recognition proteins (PRPs) can recognize such PAPMs. Each of PRRs or PRPs can recognize a proper PAMPs on the surface of the pathogens to form a complex that induce a series of immune responses such phagocytosis, nodule formation, encapsulation, proteinase cascade activation, and anti-bacterial peptides synthesis. Toll-like receptors (TLRs) are representative PRR, and TLR agonist have been developed as vaccine adjuvants because they show strong activities to immunocytes. For example, an endotoxin LPS showed strong immunity activities against TLR4 on immunocytes.

Unlike genomic DNA in higher organism such as human, bacterial DNA does not have methylated cytosine in CpG motif. The immunocytes in higher organisms can bacterial DNA in which cytosine of CpG motif is not methylated as non-self antigens. In this case, a specific receptor TLR9 recognizes the bacterial DNA. TLR9 agonists can enhance various immune responses, and TLR9 agonist such as oligo-nucleotides including CpG motif have been developed as adjuvants. However, LPS and CpG motif used as TLR agonists have very strong toxicity, causes cases side effects such as inflammatory response in the body.

### DISCLOSURE

### Technical Problem

Accordingly, the present disclosure is directed to a nucleic acid molecule, an expression vector and pharmaceutical or medicinal applications that can reduce one or more of the problems due to the limitations and disadvantages of the related art.

An object of the present disclosure is to provide an expression system that express peptides or proteins of interest efficiently without incurring complex and expensive processes.

Another object of the present disclosure is to provide a pharmaceutical composition such as adjuvant that can induce or stimulate cell-mediated immune response as well as humoral immune response.

### Solution to Problem

According to an aspect, the present disclosure provides a nucleic acid molecule comprises at least one expression control sequence comprising a viral Internal Ribosomal Entry Site (IRES) element; and at least one coding region linked operatively to the at least one expression control sequence and encoding a peptide or a protein.

In one embodiment, the nucleic acid molecule may further comprise at least one of multiple adenosines and multiple thymidines located upstream of the at least one expression control sequence.

The viral IRES element may be derived from at least one of Picornaviridae family, Togaviridae family, Dicistroviridae family, Flaviridae family, Retroviridae family and Herpesviridae family, for example, may be derived from at least one of Picornaviridae family and Dicistroviridae family.

In an exemplary embodiment, the viral IRES element derived from the Picornaviridae may be derived from at least one of Enterovirus genus, Cardiovirus genus, Apthovirus genus, Hepatovirus genus and Teschovirus genus, and the viral IRES element derived from the Dicistroviridae family may be derived from Cripavirus genus. For example, the viral IRES element may be derived from at least one of coxsackie B virus, Cricket paralysis virus, Japanese Encephalitis virus, Encephalomyocarditis virus and Sindbis virus.

In another exemplary embodiment, the at least one expression control sequence may comprise a viral 5′ untranslated region (5′ UTR). If necessary, the nucleic acid molecule may further comprise a viral 3′ Untranslated Region (3′ UTR) located downstream of the 5′ UTR, and wherein the at least one coding region is located between the 5′ UTR and the 3′ UTR.

In one embodiment, the at least one coding region may encode an antigen or fragments thereof. Alternatively, the at least one coding region encodes a protein or fragments thereof for treating disease.

In another exemplary embodiment, the at least one expression control sequence comprises a first expression control sequence having a first IRES element and a second expression control sequence located downstream of the first expression control sequence and having a second IRES element. The at least one coding region may comprise a first coding region located between the first and second expression control sequences and a second coding region located downstream of the second expression control sequence. The nucleic acid molecule may further comprise at least one of multiple adenosines or multiple thymidines located upstream of at least one of the first expression control sequence and the second expression control sequence. The first expression control sequence may comprise a first viral IRES element derived from coxsackie B virus or Cricket paralysis virus, and the second expression control sequence may comprise a second viral IRES element derived from Encephalomyocarditis virus.

Alternatively, the nucleic acid molecule may further comprise a transcription control sequence located downstream of the at least one expression control sequence, and/or a polyadenylation signal sequence or a poly adenosine sequence located downstream of the at least one coding region. The nucleic acid molecule may be RNA.

In another aspect, the present invention provides a recombination vector comprising the nucleic acid molecule described above.

In still another aspect, the present invention provides a method of stimulating, inducing and/or enhancing an immune response in a subject, the method comprising administering a pharmaceutically effective amount of the nucleic acid molecule described above to the subject. The at least one coding region of the nucleic acid molecule may encode an antigen or fragments thereof. For example, the at least one coding region may encode a peptide or a protein selected from the group consisting of a viral pathogen, a viral antigen and combination thereof.

### Advantageous Effects of Invention

In order to efficiently express a gene of interest using the conventional capping structure, there has been a problem that an expensive enzyme has to be used, and only one peptide or protein has to be expressed in one expression system.

However, the nucleic acid molecule of the present disclosure can efficiently express the desired peptide or protein in vivo without using an expensive enzyme. In addition, the present disclosure comprises IRES as an expression control sequence, so that, if necessary, the same or different peptides or proteins can be operatively linked to other IRES sequences to simultaneously produce desired peptides and proteins in a single nucleic acid molecule, and the present disclosure can increase the expression efficiency.

According to the present disclosure, a nucleic acid molecule comprising an immunogenic target sequence that can be expressed by a viral expression control sequence can enhance the immune response caused by the immunogenic substance. Therefore, the nucleic acid molecule of the present disclosure can be utilized as an adjuvant for enhancing an immune response by an immunogenic substance.

### BRIEF DESCRIPTION OF DRAWINGS

FIG. 1 is a schematic diagram illustrating components of a polynucleotide or a nucleic acid molecule that includes one expression cassette or one expression unit according to an exemplary embodiment of the present disclosure;

FIG. 2 is a schematic diagram illustrating components of a polynucleotide or a nucleic acid molecule that includes multiple expression cassettes or multiple expression units according to another exemplary embodiment of the present disclosure;

FIGS. 3A and 3B are graphs illustrating expression levels of GOI (Renilla luciferase; R/L) by administering nucleic acid molecules of RNA platform including IRES element to cells measured in accordance with an Example of the present disclosure. FIG. 3A is a graph illustrating expression levels of R/L in A204 cells and FIG. 3B is a graph illustrating expression levels of R/L in 293 cells;

FIG. 4 is a graph illustrating expression levels of GOI (Renilla luciferease, R/L) by administering nucleic acid molecules of RNA platform including IRES element to A204 cells measured in accordance with an Example of the present disclosure;

FIG. 5 is a graph illustrating expression levels of GOI (Renilla luciferase, R/L) by administering nucleic acid molecules of RNA platform including multiple IRES elements to 293T cells measured in accordance with an Example of the present disclosure;

FIGS. 6A and 6B are graphs illustrating expression levels of GOIs (Renilla luciferase, R/L; and firefly luciferase, F/L) by administering nucleic acid molecules of RNA platform including IRES element to cells measured in accordance with an Example of the present disclosure. FIG. 6A is a graph illustrating expression levels of R/L and F/L in 293T cells and FIG. 6B is a graph illustrating expression levels of R/L and F/L in Nor10 cells;

FIG. 7 is a graph illustrating MERS S protein-specific IgG1 levels measured by ELISA in accordance with an Example of the present disclosure;

FIG. 8 is a graph illustrating MERS S protein-specific IgG2c levels measured by ELISA in accordance with an Example of the present disclosure;

FIGS. 9A to 9C are graphs illustrating activated dendritic cells (CD11c+CD40+, CD11c+CD80+ and CD11c+Cd86+) derived from mice bone-marrow dendritic cells (mBMDCs), each of which CD4+ cells proliferation with regard to cell-mediated immune response, measured by flow cytometry in accordance with an Example of the present disclosure;

FIGS. 10A and 10B are graphs illustrating Th1 related cytokines, i.e. IL-12 and IL-6 production in the supernatant of mBMDCs measured by ELIS 24 hours layer in accordance with an Example of the present disclosure;

FIG. 11 is a photograph illustrating mice tissues treated with different concentrations of a nucleic acid molecules in accordance with an Example of the present disclosure;

FIG. 12 is a schematic diagram illustrating a immunization schedule of mice inoculated with MERS S protein formulated with a nucleic acid molecule in accordance with an Example of the present disclosure;

FIGS. 13A and 13B are graphs illustrating MERS S-specific IgG1 levels measured by ELISA in accordance with an Example of the present disclosure;

FIGS. 14A and 14B are graphs illustrating MERS S protein-specific IgG2c levels measured by ELISA in accordance with an Example of the present disclosure;

FIG. 15 is a graph illustrating IFN-γ producing cells in spleenocytes of mice stimulated with MERS S protein formulated with a nucleic acid molecule measured by ELISPOT in accordance with an Example of the present disclosure;

FIG. 16 is a schematic diagram illustrating a immunization schedule of mice inoculated with MERS S protein formulated with a nucleic acid molecule in accordance with an Example of the present disclosure;

FIG. 17 is a graph illustrating MERS-CoV specific neutralizing antibodies levels in serum of mice immunized with MERS S protein formulated with a nucleic acid molecule determined by Plaque Reduction Neutralization Tests (PRNT) in accordance with en Example of the present disclosure;

FIGS. 18A and 18B are graphs illustrating MERS S protein-specific IgG1 levels measured by ELISA in accordance with an Example of the present disclosure;

FIGS. 19A and 19B are graphs illustrating MERS S protein-specific IgG2c levels measured by ELISA in accordance with an Example of the present disclosure;

FIG. 20 is a schematic diagram illustrating a immunization schedule of mice inoculated with HPV protein vaccines formulated with a nucleic acid molecule in accordance with an Example of the present disclosure;

FIGS. 21A to 21C are graphs illustrating HPV protein-specific total IgG, IgG1 and IgG2 levels measured by ELISA in accordance with an Example of the present disclosure;

FIG. 22A to 22C are graph illustrating MERS S protein-specific total IgG, IgG1 and IgG2 levels at 2 weeks after 1st immunization in accordance with an Example of the present disclosure;

FIG. 23A to 23C are graph illustrating MERS S protein-specific total IgG, IgG1 and IgG2 levels at 2 weeks after 2nd immunization in accordance with an Example of the present disclosure;

FIG. 24A is a graph illustrating IFN-γ producing cells in spleenocytes of mice immunized with HPV proteins vaccine with a nucleic acid molecule measured by ELISPOT in accordance with an Example of the present disclosure and FIG. 24B illustrates IFN-γ secreting cells in the mice spleenocytes;

FIGS. 25A to 25D are graphs illustrating Th1 related cytokines, i.e. IL-2, IL-6 and IFN-γ production in the mice spleenocytes immunized with HPV protein vaccines formulated with a nucleic acid molecule measured by ELISA in accordance with an Example of the present disclosure;

FIG. 26 is a schematic diagram illustrating a immunization schedule of mice inoculated with inactivate Influenza vaccine formulated with a nucleic acid molecule in accordance with an Example of the present disclosure;

FIG. 27 is a graph illustrating influenza specific neutralizing antibody levels in serum of mice immunized with inactivated influenza vaccine formulated with a nucleic acid molecule determined by PRNT in accordance with en Example of the present disclosure;

FIG. 28 is a graphs illustrating IFN-γ producing cells in spleenocytes of mice stimulated with inactivated influenza vaccine and a nucleic acid molecule measured by ELISPOT in accordance with an Example of the present disclosure;

FIG. 29 is a graphs illustrating IL-2 producing cells in the spleenocytes of mice stimulated with inactivated influenza vaccine and a nucleic acid molecule measured by ELISPOT in accordance with an Example of the present disclosure;

FIG. 30 is a graph illustrating IL-6 production in the mice spleenocytes immunized with inactivated influenza vaccine formulated with a nucleic acid molecule measured by ELISA in accordance with an Example of the present disclosure;

FIG. 31 is a graph illustrating IFN-γ production in the mice spleenocytes immunized with inactivated influenza vaccine formulated with a nucleic acid molecule measured by ELISA in accordance with an Example of the present disclosure;

FIGS. 32A and 32B are graphs illustrating MERS S protein-IgG1 levels measured by ELISA in accordance with an Example of the present disclosure;

FIGS. 33A and 33B are graphs illustrating MERS S protein-IgG2c levels measured by ELISA in accordance with an Example of the present disclosure;

FIG. 34 is a graph illustrating neutralizing antibody level levels in serum of mice immunized with MERS S protein vaccine formulated with a nucleic acid molecule determined by PRNT in accordance with en Example of the present disclosure;

FIG. 35 is a graph illustrating IFN-γ producing cells in spleenocytes of mice immunized MERS S protein vaccine with a nucleic acid molecule measured by ELISPOT in accordance with an Example of the present disclosure;

FIG. 36 is a graph illustrating a frequencies of INF-γ, IL-2 and TNF-α producing polyfunctional CD4 T cells assayed by flow cytometry in accordance with an Example of the present disclosure;

FIGS. 37A and 37B are graphs illustrating VZV vaccine-specific IgG1 and IgG2a levels measured by ELISA in accordance with an Example of the present disclosure;

FIG. 38A is a graph illustrating IFN-γ producing cells in spleenocytes of mice immunized with VZV vaccine formulated with a nucleic acid molecule measured by ELISPOT in accordance with an Example of the present disclosure;

FIG. 38B is a graph illustrating IL-2 producing cells in spleenocytes of mice immunized with VZV vaccine formulated with a nucleic acid molecule measured by ELISPOT in accordance with an Example of the present disclosure; and

FIG. 39 is a graph illustrating neutralizing antibody titers in serum of mice immunized with VZV vaccine formulated with a nucleic acid molecule measured by FAMA in accordance with an Example of the present disclosure.

### BEST MODE FOR CARRYING OUT THE INVENTION

### Definition

As used herein, the term “amino acid” is used in the broadest sense and is intended to include not only L-amino acid but also D-amino acid, chemically-modified amino acids, and amino acid analogs.

As used herein, the term “peptide” includes any of proteins, fragments of the proteins and peptides that are isolated from naturally-occurring environment or synthesized by recombinant technique or chemical synthesis. For example, the peptides of the present disclosure may comprise, but are not limited to, at least 5, preferably 10 amino acids.

As used herein, the term “polynucleotide” or “nucleic acid” are used interchangeably, refers to polymers of any lengths of nucleotides, and includes comprehensibly DNA (i.e. cDNA) and RAN molecules. “Nucleotide”, which is a subunit of nucleic acid molecules, may comprises, but are not limited to, a deoxyribonucleotide, a ribonucleotide, a modified deoxyribonucleotide or a ribonucleotide, analogs thereof, and/or any substrates that can be incorporated into polynucleotides by DNA or RNA polymerase or synthetic reactions. Polynucleotide may comprise modified nucleotides, analogues having modified bases and/or polysaccharides such as methylated nucleotides and analogues thereof (See, Scheit, Nucleotide Analogs, John Wiley, New York, 1980; Uhlman and Peyman, Chemical Reviews, 90:543-584, 1990).

As used herein, the term “vector” means a construct or a vehicle that can be transfected or delivered into the host cells, and enables one or more genes of interest (or target genes of target sequences) to be expressed within the cells. For example, the vector may include, but are not limited to, viral vectors, DNA or RNA expression vectors, plasmid, cosmid, or phage vectors, DNA or RAN expression vectors linked to CCA (cationic condensing agents), DNA or RNA expression vectors packaged with liposomes, specific eukaryotic cells such as producer cells and the likes.

As used herein, the term “expression control sequence” (ECS) may mean nucleic acid sequences regulating or controlling transcriptional processes of the nucleic acid molecules and/or translational processes of the transcribed nucleic acid molecules. Alternatively, the term may be used to indicate nucleic acid sequences regulating or controlling the translational processes of the transcribed nucleic acid molecules. In this case, the term may be used interchangeably with the term “translation control sequence”. As used herein, the term “transcription control sequence” (TCS) means that nucleic acid sequences regulating or controlling the transcriptional process of the nucleic acid molecules. For example, the transcription control sequence comprises promoters such as a constitutive promoter or an inducible promoter, enhancers, and the likes. Each of the expression control sequence, the transcription control sequence and the translation control sequence is operatively linked to the target sequences to be expressed.

As used herein, the term “operatively linked” means a functional linkage between expression control sequence such as promoters, signal sequences, or array at transcription regulatory factor linkage sites and other nucleic acid sequences so that the expression control sequence may regulate transcriptions and/or translations of the other nucleic acid sequences.

As used herein, the term “pharmaceutically effective amount” or “therapeutically effective amount” means an amount of sufficiently accomplishing efficacy or activation of an active ingredient, a peptide or fragments thereof and/or nucleic acids encoding the peptide or fragments thereof. For example, the pharmaceutical composition containing peptides or gene delivery vehicles including nucleic acid molecules encoding the peptides.

Nucleic Acid Molecule

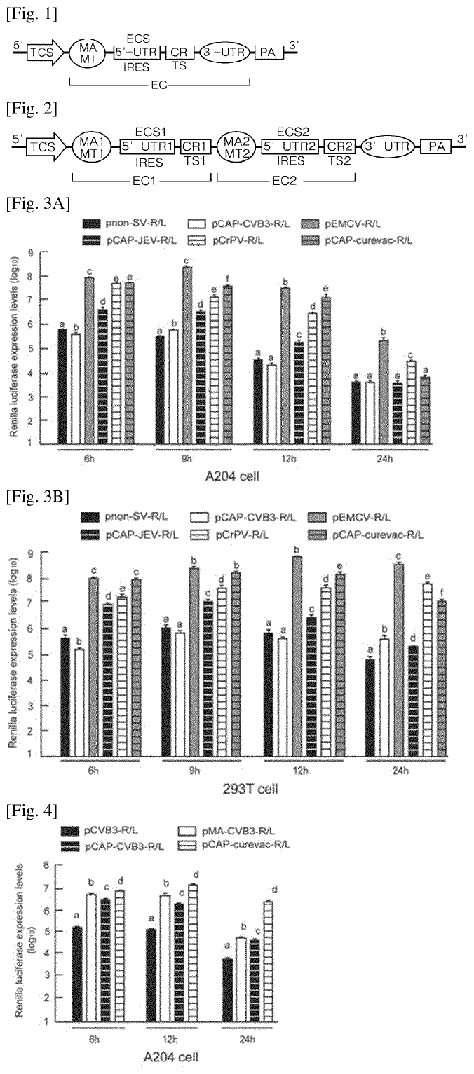

The present disclosure relates to a nucleic acid molecule or a polynucleotide comprising at least one expression control sequence having an Internal Ribosomal Entry Site (IRES) activity so as to at least one gene of interest (GOI) or target sequences. FIG. 1 is a schematic diagram illustrating components or elements of a polynucleotide or a nucleic acid molecule includes one expression cassette or expression unit according to an exemplary embodiment of the present disclosure. As illustrated in FIG. 1, the nucleic acid molecule may comprise an expression control sequence (ECS) comprising a nucleotide sequence of IRES activity and coding region (CR) linked operatively to the expression control sequence (ECS) and comprising an open reading frame (ORF) or target sequence (TS) of GOI encoding a peptide or a protein. In an exemplary embodiment, the expression control sequence (ECS) may comprise a 5′ untranslated region (5′ UTR) having IRES activity.

The coding region (CR) may be located downstream, i.e. at 3′ end of the expression control sequence (ECS), for example 5′ UTR having IRES activity and target sequence (TS) encoding the peptide of the protein. In an exemplary embodiment, the target sequence (TS) may comprises, but are not limited to, nucleotides encoding peptides or proteins with regard to immunogens, reporter peptides or proteins, drugs, pharmaceuticals, biologics and the likes.

The nucleic acid molecule may be DNA or RNA. In an exemplary embodiment, the nucleic acid molecule has an RNA platform type. In this case, the coding region (CR) may comprise transcript sequences of the GOI.

The expression control sequence (ECS) comprises nucleotide sequences having IRES activity linked operatively to the coding region (CR) inserting ORF of GOI. As described above, the expression control sequence (ECS) may have 5′ UTR structure comprising nucleotides having IRES activity. 5′ UTR is a region to which translation initiation complex bind in the course of translational processes of the peptide or the proteins expressed in the coding region (CR), and IRES is cis-acting nucleotide sequences inducing translation of the coding region (CR) by forming complex second and tertiary structure with the translation initiation complex.

In one exemplary embodiment, the expression control sequence (ECS) may comprise a viral IRES element. For example, the expression control sequence (ECS) may have 5′ UTR structure comprising viral IRES element. In an exemplary embodiment, the viral IRES element may be derived from at least one of Picornaviridae family, Togaviridae family, Dicistroviridae family, Flaviridae family, Retroviridae family and Herpesviridae family and the likes.

An IRES element has a unique secondary structure or tertiary structure and can be divided into four classes based on the molecular folding structure of the RNA and the mode of action of translation, such as that involving canonical eukaryotic initiation factors (eIFs) or specific stimulatory IRES trans-acting factors. Class I IRESs require most translational initiation factors, with the exception of eIF4E, recruit 40S ribosome complex as in the canonical scanning model, and are found in Picornaviridae family such as coxsackie B3 virus (CVB3). Class II IRESs initiate translation directly at start codons without any scanning at the 5′ end of RNA sequences and they require most eIFs, as in the case in Class I IRESs, and are found in some Picornaviridae family such as encephalomyocarditis virus (EMCV). Class III IRESs also initiate translation directly at start codons by recognizing RNA fold structures as pseudoknots without scanning but require fewer eIFs that do Class I and II IRESs and are found in Flaviviridae family such as the Japanese encephalitis virus (JEV). Class IV IRESs have a simple translational mode that does not require any eIFs. It involves only the factor 2 (eIF2) to stabilize translocation intermediates and has complicated RNA folding structure. In contrast with other IRESs, which are generally located in the 5′ UTR of RNA sequences, Class IV IRESs are found in intergenic regions (IGRs) of Dicistroviridae family such as the cricket paralysis virus (CrPV).

For example, the viral IRES element belonged to Picornaviridae may be derived from at least one of Enterovirus genus, Cardiovirus genus, Apthovirus genus, Hepatovirus genus and Teschovirus genus. In one exemplary embodiment, the viral IRES element belonged to Enterovirus genus may be derived from anyone of Enterovirus A to Enterovirus J types and/or anyone of Rhinovirus A to Rhinovirus C types.

In another exemplary embodiment, the viral IRES element belonged to Picornaviridae family may be derived from, but are not limited to, at least one of Enterovirus genus such as poliovirus (PV), Rhinovirus (RV), Coxsackie virus, for example, coxsackie B virus (CVB) such as coxsackie B3 virus (CVB3) and/or enterovirus 71 (EV71); Cardiovirus genus such as Encephalomyocarditis virus (EMCV) and/or theiler murine encephalomyelitis virus (TMEV); Apthovirus genus such as Foot-and-mouth disease virus (FMDV); Hepatovirus genus such as Hepatitis A virus (HAV); and Teschovirus genus such as porcine teschovirus (PTV), for example, PTV-1.

In an alternative embodiment, the viral IRES element belonged to Togaviridae family may be derived from, but are not limited to, at least one of Alphavirus genus such as Sindbis virus (SV). In another embodiment, the viral IRES element belonged to Dicistroviridae family may be derived from, but are not limited to, Cripavirus genus such as plautia stail intestine virus (PSIV), cricket paralysis virus (CrPV), Triatoma virus and/or Rhopalosiphum padi virus (RXPD).

In an exemplary embodiment, the viral IRES element belonged to Flaviridae family may be derived from, but are not limited to, at least one of Hepacivirus genus such as hepatitis C virus (HCV); Flavivirus genus such as Japanese encephalitis virus (JEV); Pestivirus genus such as classical swine fever virus (CSFV) and/or bovine viral diarrhea virus (BVDV). In another exemplary embodiment, the viral IRES element belonged to Retroviridae family may be derived from, but are not limited to, at least one of Gammaretrovirus genus such as friend murine leukemia virus (FMLV) and/or moloney murine leukemia virus (MMLV); and/or Alpharetrovirus genus such as rous sarcoma virus (RSV). In still another embodiment, the viral IRES element belonged to Herpesviridae family may be derived from, but are not limited to, Mardivirus such as Marek's disease virus (MDV).

In an exemplary embodiment, the viral IRES element of the expression control sequence (ECS) may be derived from at least one of Picomaviridae family and Dicistroviridae family. In this case, the viral IRES element belonged to Picomaviridae family may be derived from at least one of Enterovirus genus, Cardiovirus genus and Apthovirus genus, preferably from Enterovirus genus. For example, the viral IRES element belonged to Picornaviridae family may be derived from Enterovirus genus such as CVB3 and/or Cardiovirus genus such as EMCV. In addition, the viral IRES element belonged to Dicistroviridae family may be derived from Cripavirus genus such as PSIV and/or CrPV, preferably CrPV.

In an exemplary embodiment, the expression control sequence (ECS) may have a viral IRES element derived from, but are not limited to, at least one of SV, CVB3, EMCV, JEV and CrPV. For example, the expression control sequence (ECS) may comprise, but are not limited to, a SV-derived viral IRES element (SEQ ID NO: 1), a CVB3-derived viral IRES element (SEQ ID NO: 2), an EMCV-derived viral IRES element (SEQ ID NO: 3 and/or SEQ ID NO: 4), a JEV-derived viral IRES element (SEQ ID NO: 5), a CrPV-derived viral IRES element (SEQ ID NO: 6) and combination thereof. Alternatively, 5′ end of some viral IRES elements can be modified so as to have Cap-similar structures derived from viral proteins.

In an alternative embodiment, the nucleic acid molecule of the present disclosure may have other elements or nucleic acid sequences that can enhance expression efficiency of ORF in the coding region (CR). For example, multiple adenosines (MA) or multiple thymidines (MT) may be located adjacently to, preferably upstream (5′ end) of, the expression control sequence (ECS). In one exemplary embodiment, about 20 to about 400, preferably about 30 to about 300, more preferably about 30 to about 200, and most preferably about 30 to about 100 adenosines or thymidines may be inserted upstream of the expression control sequence (ECS) having at least one IRES element. For example, the expression control sequence (ECS) located adjacently to multiple adenosines (MA) and/or multiple thymidines (MT) comprise a viral IRES element derived from at least one of Picomaviridae, Togaviridae, Dicistroviridae, Flaviridae, Retroviridae and Herpesviridae.

In one exemplary embodiment, the viral IRES element located adjacently to multiple adenosines (MA) and/or multiple thymidines (TA) may be derived from Picomaviridae and/or Dicistroviridae. For example, the at least one of multiple adenosines (MA) and/or multiple thymidines (MT) may be inserted upstream of the expression control sequence (ECS) comprising a viral IRES element derived from, but are not limited to, Picomaviridae such as Enterovirus (e.g. CVB3) and/or Cardiovirus (e.g. EMCV) and Dicistroviridae such as Cripavirus (e.g. CrPV).

The coding region (CR) may comprise nucleotides of ORFs encoding corresponding peptides or proteins and linked operatively to the expression control sequence (ECS). The coding region (CR) may be located downstream (3′ end) of the expression control sequence (ECS). In one exemplary embodiment, the coding region (CR) may comprise ORFs encoding anyone of reporter peptides/proteins, marker or selection peptides/proteins, antigens, antibodies, drugs, pharmaceuticals, biologics, fragments thereof, variants thereof and/or derives thereof. For example, the coding region (CR) may comprise ORFs encoding peptides or proteins such as antigens or epitopes thereof in case the coding region (CR) encodes an immunogenic peptides or proteins.

In one exemplary embodiment, the ORF in the coding region (CR) may encode luciferases such as Renilla luciferease (SEQ ID NO: 16 and/or SEQ ID NO; 17) and/or Firefly luciferease (SEQ ID NO: 18), green fluorescent protein (GFP), enhanced green fluorescence protein (EGFP) and/or beta-galactosidase in case the coding region (CR) encodes the reporter proteins or peptides. The ORF encoding the marker or selection proteins or peptide may comprise nucleotides encoding alpha-globin, galactokinase, xanthine guanine phosphoribosyl transferase, and the likes. Other ORFs encoding other report proteins/peptides and/or marker or selection proteins/peptides may be inserted within the coding region (CR).

In another exemplary embodiment, the coding region (CR) may comprise ORFs encoding antigens, fragment thereof, variants or derivatives thereof. For example, the antigens can be expressed from the coding region (CR) may comprise tumor antigens, animal antigens, vegetation antigens, viral antigens, bacterial antigens, fugal antigens, protozoan antigens, autoimmune antigens and/or allergic antigens. Preferably, the antigens may have secreted forms of surface antigens of tumor cells, viral pathogens, bacterial pathogens, fungal antigens and/or protozoan antigens.

If necessary, the antigens may be in the nucleic acid molecule according to the present disclosure, or as heptene bound to an appropriate carrier. Other antigenic components, for example, inactivated or attenuated pathogens may be used.

Particular preferred tumor antigen expressed from the coding region (CR) may be tumor-specific surface antigens (TSSA). Such tumor antigens may be, but are not limited to, selected from the group consisting of p53, CA125, EGFR, Her2/neu, hTERT, PAP, MAGE-A1, MAGE-A3, Mesothelin, MUC-1, GP100, MART-1, Tyrosinase, PSA, PSCA, PSMA, STEAP-1, VEGF, VEGFR1, VEGFR2, Ras, CEA or WT1, and preferably from PAP, MAGE-A3, WT1, and MUC-1.

In another exemplary embodiment, the pathogenic antigens may be expressed from the coding region (CR) is originated from pathogenic organisms inducing immune responses by mammalian individuals, particularly by humans. For example, the pathogenic antigens may be originated from bacterial, viral or protozoan (multi-cellular) pathogenic organisms. In one exemplary embodiment, the pathogenic antigens may be surface antigens located on the surface of organisms such as viruses, bacteria or protozoa, for example, proteins (or fragment of proteins such as external parts of the surface antigens).

In another exemplary embodiment, the pathogenic antigens may be expressed form the coding region (CR) is peptide or protein antigens originated from infectious-diseases associated pathogens. The pathogens associated with the infectious diseases may comprise, but are not limited to, influenza virus, Respiratory syncytial virus (RSV), Herpes simplex virus (HSV), Human papillomavirus (HPV), Human immunodeficiency virus (HIV), Plasmodium genus, Staphylococcus genus, Dengue viruses, Chlamydia trachomatis, Cytomegalovirus (CMV), Hepatitis B Virus (HBV), Mycobacterium tuberculosis, Rabies virus, Yellow fever virus, Middle East respiratory syndrome coronavirus (MERS-CoV), and/or zika virus.

In one exemplary embodiment, the coding region (CR) may comprise, but are not limited to, an ORF (SEQ ID NO: 19) encoding a spike peptide in MERS-CoV or an ORF (SEQ ID NO: 20) encoding a fragment a spike peptide in MERS-CoV, an ORF encoding L1 region or its fragments in HPV, for example, an ORF (SEQ ID NO: 21) encoding L1 region or its fragment in HPV-16, an ORF (SEQ ID NO: 22) encoding L1 region or its fragment in HPV-18, an ORF (SEQ ID NO: 23) encoding haemagglutin (HA) or its fragment in influenza viruses, an ORF (SEQ ID NO: 25) encoding gE or its fragment in Varicella-Zoster virus (VZV) and/or equivalent nucleotides thereof.

In one exemplary embodiment, the peptide or protein may be expressed from the coding region (CR) is an immunogenic peptide or protein such as antigens.

In one exemplary embodiment, the nucleic acid molecule of the present disclosure may be an RNA platform. In this case, the nucleic acid molecule of the present disclosure may utilized as RNA vaccines that can be injected into an individual or a subject when the coding region (CR) comprises ORFs encoding peptides and/or proteins such as pathogenic antigens that can induce immune responses in the individual. RNA expression platforms have many advantages over DNA expression platforms.

RNA expression platforms have a high degree of safety because they do not require nuclear entry and host chromosomal integration, and lack an antibiotic gene, thus avoiding antibiotic resistance. Besides, RNA expression platforms show paradoxically fast degradation, thus avoiding the immune-toxicity caused by repeated injections. Also, RNA expression platforms show convenient production in vitro because of the lack of any need for unnecessary biological processes, such as mass cell culture and live pathogen culture, thereby escaping the requirement for biological facilities such as bioreactors. In addition, the RNA expression platforms afford a well-balanced induction of immune responses, such as T-helper cell 1 (Th1) and T-helper cell 2 (Th2) activation, as well as humoral and cellular responses because of the characteristic ability of RNAs to activate innate immune pathways.

In another exemplary embodiment, the coding region (CR) may comprise ORFs encoding therapeutic peptides and/or proteins with regard to curing or treating diseases. In this case, the nucleic acid molecule of the present disclosure may be utilized as gene therapy and/or an adjuvant enhancing immune responses with regard to treating diseases.

In an exemplary embodiment, the therapeutic peptide or the protein with regard to treating diseases may comprise, but are not limited to, therapeutic peptides or proteins used for treating metabolic or endocrine disorders; therapeutic peptides or proteins used for treating blood disorders, circulatory system disorders, respiratory system disorders, cancer or tumor disorders, infections disorders or immune-deficiency; therapeutic peptides or proteins used for treating hormone replacement therapies; therapeutic peptides or proteins used for differentiating reversely somatic cells into omni- or pluri-potent stem cells; therapeutic peptides or proteins selected from adjuvant or immune-stimulatory proteins; and/or antibodies. Such peptides or proteins may constitute pharmaceutically active ingredients among a pharmaceutical composition as described below.

For example, the peptides or proteins used for treating metabolic or endocrine disorders may comprise, but are not limited to, Bone morphogenetic protein (BMP), Epidermal growth factor (EGF), Fibroblast Growth Factor (FGF), Insulin-like growth factor 1 (IGF-1), IGF-1 analog and the likes. These and other proteins are understood to be therapeutic, as they are meant to treat the subject by replacing its defective endogenous production of a functional protein in sufficient amounts. Accordingly, such therapeutic proteins are typically mammalian, in particular human proteins.

Also, adjuvant or immune-stimulatory proteins may be used to induce or improve an immune response in an individual to treat a particular disease or ameliorate condition of the individual. In a still another exemplary embodiment, the therapeutic peptide or the protein with regard to adjuvant or immune-stimulatory proteins may comprise, but are not limited to, human adjuvant proteins, in particular the pattern recognition receptors such as TLR1, TLR2, TLR3, TLR4, TLR5, TLR6, TLR7, TLR8, TLR9, TLR10, TLR11; NOD1, NOD2, NOD3, NOD4, NOD5, NALP1, NALP2, NALP3, NALP4, NALP5, NALP6, NALP6, NALP7, NALP7, NALP8, NALP9, NALP10, NALP11, NALP12, NALP13, NALP14, I IPAF, NAIP, CIITA, RIG-I, MDA5 and LGP2.

Pathogenic adjuvant proteins, typically comprise any pathogenic adjuvant protein that is capable of eliciting an innate immune response in a mammal, more preferably selected from pathogenic adjuvant proteins derived from bacteria, protozoa, viruses or fungi and the likes, e.g. bacterial adjuvant proteins, protozoan adjuvant proteins (e.g., profiling-like protein of Toxopolasm gondi), viral adjuvant proteins, or fungal adjuvant proteins.

Particularly, bacterial adjuvant proteins may be selected from the group consisting of bacterial heat shock proteins or chaperons, including Hsp60, Hsp70, Hsp90, Hsp100; OmpA (Outer membrane protein) from gram-negative bacteria; bacterial porins, including OmpF; bacterial toxins, including pertussis toxin (PT) from Bordetella pertussis, pertussis adenylate cyclase toxin CyaA and CyaC from Bordetella pertussis, PT-9K/129G mutant from pertussis toxin, pertussis adenylate cyclase toxin CyaA and CyaC from Bordetella pertussis, tetanus toxin, cholera toxin (CT), cholera toxin B-subunit, CTK63 mutant from cholera toxin, CTE112K mutant from CT, Escherichia coli heat-labile enterotoxin (LT), B subunit from heat-labile enterotoxin (LTB) Escherichia coli heat-labile enterotoxin mutants with reduced toxicity, including LTK63, LTR72; phenol-soluble modulin; neutrophil-activating protein (HP-NAP) from Helicobacter pylori; Surfactant protein D; Outer surface protein A lipoprotein from Borrelia burgdorferi, Ag38 (38 kDa antigen) from Mycobacterium tuberculosis; proteins from bacterial fimbriae; Enterotoxin CT of Vibrio cholerae, Pilin from pili from gram negative bacteria, and Surfactant protein A; and the likes, or any species homolog of any of the above bacterial (adjuvant) proteins.

Bacterial adjuvant proteins may also comprise bacterial flagellins. In one embodiment, bacterial flagellins may be selected from flagellins from organisms comprising, but are not limited to, Agrobacterium, Aquifex, Azospirillum, Bacillus, Bartonella, Bordetella, Borrelia, Burkholderia, Campylobacter, Caulobacte, Clostridium, Escherichia, Helicobacter, Lachnospiraceae, Legionella, Listeria, Proteus, Pseudomonas, Rhizobium, Rhodobacter, Roseburia, Salmonella, Serpulina, Serratia, Shigella, Treponema, Vibrio, Wolinella, Yersinia, more preferably from flagellins from the species including, without being limited thereto, Agrobacterium tumefaciens, Aquifex pyrophilus, Azospirillum brasilense, Bacillus subtilis, Bacillus thuringiensis, Bartonella bacilliformis, Bordetella bronchiseptica, Borrelia burgdorferi, Burkholderia cepacia, Campylobacter jejuni, Caulobacter crescentus, Clostridium botulinum strain Bennett clone 1, Escherichia coli, Helicobacter pylori, Lachnospiraceae bacterium, Legionella pneumophila, Listeria monocytogenes, Proteus mirabilis, Pseudomonas aeroguinosa, Pseudomonas syringae, Rhizobium meliloti, Rhodobacter sphaeroides, Roseburia cecicola, Rosebuds hominis, Salmonella typhimurium, Salmonella bongos, Salmonella typhi, Salmonella enteritidis, Serpulina hyodysenteriae, Serratia marcescens, Shigella boydii, Treponema phagedenis, Vibrio alginolyticus, Vibrio cholerae, Vibrio parahaemolyticus, Wolinella succinogenes and Yersinia enterocolitica.

Protozoan (adjuvant) proteins are a further example of pathogenic adjuvant proteins. Protozoan (adjuvant) proteins may be selected from any protozoan protein showing adjuvant character, more preferably, from the group consisting of, but are not limited to, Tc52 from Trypanosoma cruzi, PFTG from Trypanosoma gondii, Protozoan heat shock proteins, LeIF from Leishmania spp., profiling-like protein from Toxoplasma gondii, and the likes.

Viral (adjuvant) proteins are another example of pathogenic adjuvant proteins. In this context, viral (adjuvant) proteins may be selected from any viral protein showing adjuvant character, more preferably, from the group consisting of, but are not limited to, Respiratory Syncytial Virus fusion glycoprotein (F-protein), envelope protein from MMT virus, mouse leukemia virus protein, Hemagglutinin protein of wild-type measles virus, and the likes.

Fungal (adjuvant) proteins are even a further example of pathogenic adjuvant proteins. In the context of the present invention, fungal (adjuvant) proteins may be selected from any fungal protein showing adjuvant character, more preferably, from the group consisting of, fungal immunomodulatory protein (FIP; LZ-8), and the likes.

Besides, adjuvant proteins may furthermore be selected from the group consisting of, Keyhole limpet hemocyanin (KLH), OspA, and the likes.

In a further embodiment, therapeutic proteins may be used for hormone replacement therapy, particularly for the therapy of women in the menopause. These therapeutic proteins are preferably selected from oestrogens, progesterone or progestins, and sometimes testosterone.

Furthermore, therapeutic proteins may be used for reprogramming of somatic cells into pluri- or omnipotent stem cells. For this purpose several factors are described, particularly Oct-3/4, Sox gene family (Sox1, Sox2, Sox3, and Sox15), Klf family (Klf1, Klf2, Klf4, and Klf5), Myc family (c-myc, L-myc, and N-myc), Nanog, and LIN28.

As mentioned above, also therapeutic antibodies are defined herein as therapeutic proteins. These therapeutic antibodies are preferably selected from antibodies which are used inter alia for the treatment of cancer or tumor diseases.

In one exemplary embodiment, the coding region (CR) may comprise ORFs corresponding to the pharmaceutically active ingredient in the pharmaceutical composition, i.e. encoding the pharmaceutically active ingredients or fragments thereof. For example, the coding region (CR) may comprise ORFs encoding antigens or antibodies or fragments thereof when the pharmaceutically active ingredients comprise peptides or proteins such as the antigens or the antibodies.

There is no limitation in the length of the ORFs in the coding region (CR), and the expression efficiency depending on the ORF length is not considered in developing a nucleic acid molecule, a recombinant vector, and pharmaceutical or medicinal applications for preventing or treating diseases using the molecule. Codon usage is not considered in developing human vaccines or gene therapies because codon usage basis in human has not affect on common peptides/proteins expression significantly. But, it may be preferable that start codon have Kozak sequence and nucleotides adjacent to termination codon may be optimized. If necessary, the third codon among GOI or its transcript mRNA codon to be expressed may be changed “G/C” without changing amino acid so that mRNA may have improved stability.

The nucleic acid molecule may comprise at least one Cloning Site, preferably Multiple Cloning Site (MCS) for inserting the coding region (CR) therein. The at least one Cloning Site may comprise at least one restriction endonuclease recognition site and/or site cut by at least one restriction endonuclease. In one embodiment, the restriction endonuclease may comprise artificially engineered restriction endonuclease (e.g. zinc finger nuclease or restriction endonuclease based on DNA binding site of TAL effector or PNA-based PNAzymes) as well as naturally-occurring endonuclease found in bacterial or archaebacteria. For example, the naturally-occurring restriction endonuclease may be classified into 1) Type I endonuclease (cuts sites spaced apart from recognition site and requires ATP, S-adenosyl-L-methionine and Mg2+), 2) Type II endonuclease (cuts within or spaced apart from recognition site and most requires Mg2+), 3) Type III endonuclease (cuts apart from recognition site and requires only ATP without hydrolysis of ATP), 4) Type IV endonuclease (targets modified sites as methylation, hydroxyl methylation or glucosyl-hydroxyl methylation), and 5) Type V endonuclease (e.g. CRISPR cas9-mRNA complex).

For example, the following restriction endonuclease recognition site and/or cutting site may be used: 5′-ATCGAT-3′(AngI), 5′-AGGCCT-3′(AatI), 5′-TGATCA-3′(AbaI), 5′-GGATCC-3′(BamHI), 5′-GCAGC(N)8-3′(BbvI), 5′-(N)10CGA(N)6TGC(N)12-3′(BcgI), 5′-(N)8GAG(N)5CTC(N)13-3′(BplI), 5′-GTCTC(N)-3′(BsmAI; Alw26I), 5′-ACTGGN-3′(BsrI), 5′-ATCGAT-3′(ClaI), 5′-CTCTTCN-3′(EarI), 5′-CTGAAG(N)16-3′(Eco57I), 5′-GAATTC-3′(EcoRI), 5′-CCWGG-3′(EcoRII?; W is A or T), 5′-GATATC-3′(EcoRV), 5′-GGATG(N)9-3′(FokI), 5′-GGCC-3′(HaeIII?), 5′-AAGCTT-3′(HindIII?), 5′-CCGG-3′(HpaIII?), 5′-GGTGA(N)8-3′(HphI), 5′-GGTACC-3′(KpnI), 5′-GATC-3′(MboI), 5′-ACGCGT-3′(MluI), 5′-GCCGGC-3′(NaeI), 5′-GATATG-3′(NdeII?), 5′-GCCGGC-3′(NgoMIV?), 5′-CATG-3′(NlaIII?), 5′-GCGGCCGC-3′(NotI), 5′-TTAATTAA-3′(PacI), 5′-CTGCAG-3′(PstI), 5′-GAGCTC-3′(SacI), 5′-CCGCGG-3′(SacII?), 5′-GTCGAC-3′(SalI), 5′-GCATC(N)5-3′(SfaNI), 5′-CCCGGG-3′(SmaI), 5′-TCGA-3′(TaqI), 5′-TCTAGA-3′(XbaI), 5′-CTCGAG-3′(XhoI) and combination thereof. In one exemplary embodiment, the cloning site may comprise multi-closing site (MCS).

Besides, the nucleic acid molecule of the present disclosure may comprise optionally 3′ UTR so as to enhance expression efficiency of the ORF in the coding region (CR) in case the expression control sequence (ECS) has 5′ UTR including an IRES element. In an embodiment, the coding region (CR) may be located between 5′ UTR and 3′ UTR. 3′ UTR may enhance translation efficiency of GOI or its transcript together with 5′ UTR and has a significant role in stabilizing transcript mRNA in cells. In one exemplary embodiment, 3′ UTR may be derived from viral sources as 5′ UTR including IRES elements. 3′ UTR may be derived from identical or different viruses from 5′ UTR.

In one exemplary embodiment, a viral 3′ UTR may be derived from at least one of Picornaviridae family, Togaviridae family, Dicistroviridae family Flaviridae family, Retroviridae family and Herpesviridae family.

For example, viral 3′ UTR belonged to of Picornaviridae family may be derived from, but are not limited to, at least one of Enterovirus genus (e.g. PV, RV, coxsackie virus such as CVB3, and/or EV71), Cardiovirus genus (e.g. EMCV and/or TMEV), Apthovirus genus (e.g. FMDV), Hepatovirus genus (e.g. HAV) and Teschovirus genus (e.g. PTV such as PTV-1).

In addition, viral 3′ UTR belonged to Togaviridae family may be derived from, but are not limited to, Alphavirus genus (e.g. SV), and viral 3′ UTR belonged to Dicistroviridae family may be derived from, but are not limited to, Cripavirus genus (e.g. PSIV, CrPV, Triatoma virus and/or RXID). In an alternative embodiment, viral 3′ UTR belonged to Flaviridae family may be derived from, but are not limited to, Hepacivirus genus (e.g. HCV), Flavivirus genus (e.g. JEV), Flavivirus genus (e.g. JEV) and/or Pestivirus genus (e.g. CSFV and/or BVDV). In another embodiment, viral 3′ UTR belonged to Retroviridae family may be derived from, but are not limited to, Alpharetrovirus genus (e.g. RSV), and viral 3′ UTR belonged to Herpesviridae family may be derived from, but are not limited to, Mardivirus genus (e.g. MDV).

In one exemplary embodiment, the viral 3′ UTR may comprise, but are not limited to, 3′ UTR derived from SV (SEQ ID NO: 7), 3′ UTR derived from CVB3 (SEQ ID NO: 8), 3′ UTR derived from EMCV (SEQ ID NO: 9) and/or 3′ UTR derived from JEV (SEQ ID NO: 10).

Besides, the nucleic acid molecule of the present disclosure may further comprise transcription control sequence (TCS) located adjacently to the expression control sequence (ECS) for promoting transcription of thereof. For example, the transcription control sequence (TCS) may be located upstream (5′ end) of the expression control sequence (ECS). Such transcription control sequence (TCS) is not limited to specific elements, and will be described in the following recombinant vector section in more detail.

Further, the nucleic acid molecule may other elements for inducing expression of ORFs in the coding region (CR) as well as the expression control sequence (ECS), the coding region (CR), 3′ UTR and the transcription control sequence (TCS). In one exemplary embodiment, the nucleic acid molecule may have Kozak sequence/element inserted between the expression control sequence (ECS) (e.g. 5′ UTR having IRES element) and the start codon of the coding region (CR). If necessary, the nucleic acid molecule further comprises downstream hairpin structure (DLP) at 3′ end of the expression control sequence (ECS). For example, DLP element or sequence (e.g. SEQ ID NO: 11) derived from SV may be inserted between 5′ UTR and the coding region (CR) when 5′ UTR derived from SV (e.g. SEQ ID NO: 1) as the expression control sequence is applied.

In another alternative embodiment, nucleotides as start codon (e.g. CCTGCT) and/or another recognition sequence (e.g. ATGGCAGCTCAA)(SEQ ID NO: 29) for enhancing expression of GOI may be inserted downstream of the expression control sequence.

Also, a polyadenylation signal sequence and/or polyadenosine sequence (PA) may be inserted downstream of the coding region (CR), or 3′ UTR in case of using 3′ UTR so as to stabilized the transcribed nucleic acid molecule and further enhance translation efficiency of ORFs in the coding region (CR). For example, the polyadenosine sequence (PA) may comprise about 25 to about 400, preferably about 30 to about 400, more preferably about 50 to about 250, and most preferably about 60 to about 250 adenosine nucleotides when the nucleic acid molecule of the present disclosure comprise RNA transcript nucleotides.

In still another exemplary embodiment, polyadenylation signal sequences may be located downstream of the coding region (CR) in case the nucleic acid molecule comprises DNA platform nucleotides. The polyadenylation signal sequence may have common structure of 5′-NNUANA-3′ motif (wherein N is any base or nucleotide of adenine/adenosine, cytosine/cytidine, thymine/thymidine, guanine/guanidine and uracil/uridine). For example, the polyadenylation signal sequence may common structures such as 5′-AAUAAA′-3′ or 5′-AUUAAA-3′. For example, the polyadenylation signal sequence may be derived from, but are not limited to, SV40, human growth factor (hGH), bovine growth factor (BGH) and/or rabbit beta-globin (rbGlob).

In FIG. 1, the nucleic acid molecule has only expression cassette (EC) including only one expression control sequence (ECS) and only one coding region (CR). In a different embodiment, a nucleic acid molecule of the present disclosure may comprises multiple expression control sequences having IRES elements and multiple coding regions encoding peptides or proteins that can be expressed by at least one of multiple expression control sequences. FIG. 2 is a schematic diagram illustrating components or elements of a polynucleotide or a nucleic acid molecule that includes multiple expression cassettes or multiple expression units according to another exemplary embodiment of the present disclosure.

As illustrated in FIG. 2, the nucleic acid molecule according to another embodiment of the present disclosure comprises two expression cassettes “EC1” and “EC2”. The first expression cassette “EC1” comprises a first expression control sequence “ECS1” (e.g. 5′ UTR 1) having an IRES element and a first coding region “CR1” linked operatively to the first expression control sequence “ECS1” and comprising ORF as a first target sequence “TS1”. The second expression cassette “EC2” comprises a second expression control sequence “ECS2” (e.g. 5′ UTR 2) having an IRES element and a second coding region “CR2” linked operatively to the second expression control sequence “ECS2” and comprising ORF as a second target sequence “TS2”. In one exemplary embodiment, the second expression control sequence “ECS2” may be located downstream of the first expression control sequence “ECS1”, the first coding region “CR1” may be located between the first expression control sequence “ECS1” and the second expression control sequence “ECS2”, and the second coding region “CR2” may be located downstream of the second expression control sequence “ECS2”.

In one exemplary embodiment, each the first expression control sequence “ECS1” and the second expression control sequence “ECS2” may 5′ UTR having the viral IRES elements as described above with reference with FIG. 1. The first expression control sequence “ECS1” and the second expression control sequence “ECS2” may have viral IRES elements derived from identical source. Alternatively, the first expression control sequence “ECS1” and the second expression control sequence “ECS2” may have viral IRES elements derived from different sources.

In one alternative embodiment, the nucleic acid molecule may further comprise multiple adenosines or multiple thymidines inserted adjacently to, for example, upstream (5′ end) of at least one of the multiple expression control sequences “ECS1” and “ECS2”. In FIG. 2, multiple adenosines or multiple thymidines “MA1/MT1” are inserted upstream of the first expression control sequence “ECS1” that is located adjacently to the transcription control sequence (TCS), and anther multiple adenosines or multiple thymidines “MA2/MT2” are inserted upstream of the second expression control sequence “ECS2”. However, multiple adenosines or multiple thymidines, each of which enhances respective ORF in the coding regions “CR1” and “CR2”, may be inserted adjacently to, preferably upstream of, at least one of the first and second expression control sequences “ECS1” and “ECS2”.

In one exemplary embodiment, at least one of the first and second expression control sequences “ECS1” and “ECS2” may comprise a viral IRES element. Concretely, the first and/or second expression control sequences “ECS1” and “ECS2” may comprises a viral IRES element derived from at least one of Picornaviridae family, Togaviridae family, Dicistroviridae family, Flaviridae family, Retroviridae family and Herpesviridae family.

In an embodiment, the first and/or second expression control sequences “ECS1” and “ECS2” may comprise a viral IRES element derived from at least one of Picornaviridae family and Dicistroviridae family. For example, the first and/or second expression control sequences “ECS1” and “ECS2” including a viral IRES element belonged to Picornaviridae family may comprise a viral IRES element derived from at least one of Enterovirus genus, Cardiovirus genus, Apthovirus genus, Hepatovirus genus and Teschovirus genus, and the viral IRES element derived from the Dicistroviridae family is derived from Cripavirus genus, preferably Enterovirus genus. For example, the first and/or second expression control sequences “ECS1” and “ECS2” including a viral IRES element belonged to Picornaviridae family may comprise a viral IRES element derived from Enterovirus genus (e.g. coxsackie virus such as CVB3) and/or a viral IRES element derived from Cardiovirus genus (e.g. EMCV). In another embodiment, the first and/or second expression control sequences “ECS1” and “ECS2” including a viral IRES element belonged to Dicistroviridae family may comprise a viral IRES element derived from Cripavirus genus (e.g. PSIV and/or CrPV).

Besides, the coding region comprise a first coding region “CR1” located between the first and second expression control sequences “ECS1” and “ECS2”, and a second coding region “CR2” located downstream of the second expression control sequence “ECS2”. Each of the first and second coding regions “CR1” and “CR2” may comprise ORF of GOI or its transcript encoding peptides or proteins. For example, each of the first and second coding regions “CR1” and “CR2” may comprise ORFs encoding reporter peptides or proteins, marker or selection peptides or proteins, antigens and/or peptides or proteins with regard to treating diseases. In one exemplary embodiment, the first coding region “CR1” may be linked operatively to the first expression control sequence “ECS1” (e.g. 5′ UTR 1), and the second coding region “CR2” may be linked to operatively to the second expression control sequence “ECS2” (e.g. 5′ UTR 2).

In one exemplary embodiment, each of the first target sequence “TS1” as an ORF in the first coding region “CR1” and the second target sequence “TS2” as anther ORF in the second coding region “CR2” may have ORFs encoding different peptides or proteins. In this case, the nucleic acid molecule can express different peptides or proteins. For example, when each of the first and second coding regions “CR1” and “CR2” comprises ORFs encoding different antigens or fragment thereof one another, the nucleic acid molecule or the recombinant vector comprising the molecule can express different antigens and can be utilized genetic vaccines for preventing multiple diseases. In another embodiment, when each of the first and second coding regions “CR1” and “CR2” comprises ORFs encoding different therapeutic peptides or proteins, the nucleic acid molecule or the recombinant vector comprising the molecule can be utilized for treating or curing multiple diseases. Alternatively, each of the first target sequence “TS1” as an ORF in the first coding region “CR1” and the second target sequence “TS2” as anther ORF in the second coding region “CR2” may have ORFs encoding the same peptides or proteins.

Similar to the nucleic acid molecule illustrated in FIG. 1, the nucleic acid molecule illustrated in FIG. 2 including multiple expression control sequences “ECS1” and “ECS2” and multiple coding regions “CR1” and “CR2”, may comprise further nucleotides for expression of “GOT 1” and “GOI 2” in each of the coding regions “CR1” and “CR2”. For example, the nucleic acid molecule may further comprise 3′ UTR when the first and/or second expression control sequences “ECS1” and “ECS2” includes 5′ UTR having IRES element such as a viral IRES element. 3′ UTR may be located downstream of the second coding region “CR2”. The 3′ UTR may comprise the viral 3′ UTR as described above.

For example, 3′ UTR may be derived from the same sources at least one of 5′ UTR 1 in the first expression control sequence “ECS1” or 5′ UTR 2 in the second expression control sequence “ECS2”. In an exemplary embodiment, 3′UTR may be derived from, but are not limited to, the same source of 5′ UTR 1.

In addition, the nucleic acid molecule in FIG. 2 may further comprise a transcription control sequence (TCS) adjacently to, preferably upstream of the first expression control sequence “ECS1”, and Kozak sequence between each of the expression control sequences “ECS1” and “ECS2” and each of the coding sequences “CR1” and “CR2”. Besides, if necessary, the nucleic acid molecule may further comprise DLP sequence, start codon sequences and/or recognition sequences for enhancing expression of “GOI 1” and “GOI 2” downstream of each of the expression control sequences “ECS1” and “ECS2”. Also, in one exemplary embodiment, the nucleic acid molecule may further comprise polyadenylation signal sequence or poly adenosine sequences (PA) downstream of the second coding region “CR2”, or 3′ UTR if the 3′ UTR is inserted.

While FIG. 2 shows two expression control sequences “ECS1” and “ECS2” and two coding regions “CR1” and “CR2”, the nucleic acid molecule may have three or more expression control sequences and/or coding regions.

Recombinant Vector and Pharmaceutical Application

The nucleic acid molecules shown in FIGS. 1 and 2 may be inserted into a vector. The vector may have another expression control sequence linked operatively to the nucleic acid molecules. If necessary, the nucleic acid molecule may be linked to another nucleic acid molecule so as to encode fused peptides or fused proteins.

For example, the vector may include, but are not limited to, viral vectors, DNA or RNA expression vectors, plasmid, cosmid, or phage vectors, DNA or RNA expression vectors linked to CCA (cationic condensing agents), DNA or RNA expression vectors packaged with liposomes, specific eukaryotic cells such as producer cells and the likes.

In one exemplary embodiment, the nucleic acid molecules of the present disclosure are construed in order to transfect into mammalian cells and express peptides or proteins of interest. Such a construction is particularly useful for the purposes of treatment. There are many processes to express a nucleic acid molecule in the host cells and it is possible to adopt any appropriate processes. For example, the nucleic acid molecules of the present disclosure may be inserted into viral vectors such as adenovirus, adeno-associated virus, retrovirus, vaccinia virus, Lentivirus, baculovirus or other pox viruses (e.g. avian pox virus), and the likes. It has already been well-known that techniques of inserting nucleic acid molecules, for example DNA, into such vectors. It is possible to insert additionally targeting moieties such as selection marker genes for making easy certification or selection for the transfected cells and/or genes encoding ligands acting as a receptor to a particular target cell in the retrovirus vector. Targeting may be performed by known processes using specific antigens.

It is possible to use plural vectors that are commercially available and known to in the art for the purposes of the present disclosure. Selecting appropriate vectors will be mainly dependent upon the sizes of the nucleic acid molecules to be inserted into the vectors and specific host cells transfected with the vectors. Each vector contains various components, depending upon its functions (amplification and/or expression of foreign polynucleotides) and compatibilities to the specific host cells having thereof.

For example, the recombinant vector of the present disclosure may comprise another expression control sequences, which may have an effect on the expression of the peptides or proteins, such as a initiation codon, a termination codon, a polyadenylation signal sequences, enhancers, signal sequences for membrane-targeting or secretions, and the likes. The polyadenylation sequence makes transcript safety increase and facilitates cytoplasm transportation of the transcript. Enhancer sequences are nucleic acid sequences which are located at various sites with regard to transcription control sequence, e.g. promoter and increase transcription activity compared to a transcription activity by the promoter without the enhancer sequences. Signal sequences comprise, but are not limited to, PhoA signal sequence, OmpA signal sequence and the likes in case the host cell is bacteria in Escherichia spp., α-amylase signal sequence, subtilisin sequence and the likes in case the host cell is bacteria in Bacillus spp., MF-α signal sequence, SUC2 signal sequence and the likes in case the hose cell is yeast, and insulin signal sequence, α-interferon signal sequence, antibody molecule signal sequence and the likes in case the host cell is mammals.

A category of vector is a ‘plasmid’ which refers to a circular, double-stranded DNA loop into which additional nucleic acid molecule may be ligated. Another category of vector is a phage vector. Still another category of vector is viral vectors into which additional nucleic acid molecule may be ligated into the viral genome. Specific vectors can replicate autonomously into the host cells having the transfected the vectors (e.g. viral vectors and episome mammalian vectors having bacterial replication origins). Other vectors (e.g. non-episome mammalian vectors) may be integrated into the genome of a host cell as they transfect the host cell, and thereby, being replicated together with the genome of the host cell. Besides, specific vectors may direct the expression of genes operatively linked to the vectors. Such vectors are referred herein as a “recombinant expression vector (or, shortly, “recombinant vector”). Generally, the expression vectors, which may be useful for recombinant DNA technologies, exist as a shape of plasmid.

Constitutively or inducible promoters can be used as the transcription control sequence (TCS) in the present disclosure. Plural promoters that recognized by various possible host cells have been widely known in the art. Selected promoters may be linked operatively to the nucleic acid molecule having at least one coding region “CR”, “CR1” and “CR2” comprising ORF of appropriate GOI by removing the promoters from suppliers nucleic acid molecule through restriction enzyme digestions and then inserting the isolated promoter sequences into the selection vectors. It is possible to direct amplification and/or expression of the target genes using both natural promoter sequences and a plurality of foreign promoters. But, foreign promoters are generally more preferable to the natural targeting polypeptide promoters because the foreign promoters allows much transcription and high yield of the expressed target genes compared to the natural targeting polypeptide promoters.

Besides, when the recombinant vector of the present disclosure is a replicable repression vector, it may comprise a replication origin, which is a specific nucleic acid sequence for initiating replication. In addition, the recombinant vectors may comprise sequences encoding selectable markers. The selectable markers are intended to screen transfected cells by the vectors and markers giving selectable phenotypes such as drug resistances, nutritional requirements, cytotoxic agent resistances, or expressions of surface proteins may be used. The vectors of the present invention may comprise antibiotics resistant genes which have been conventionally used in the art, for example, ampicillin, gentamicin, carbenicillin, chloramphenicol, streptomycin, kanamycin, geneticin, neomycin, and tetracycline resistant genes as selectable markers. It is possible to screen the transfected cells because only cells expressing the selectable markers can survive in an environment of treating elective agents. Representative example of the selectable markers may comprise an auxotrophic marker, ura4, leu1, his3 and the likes, but the selectable markers can be used in the present invention is not limited to such an example.

It is possible to use any host cells known in the art as long as the host cells make the vectors stably and continuously clone and express.

The vector injected into the host cells may be expressed within the cell in which large amount of recombinant peptides or proteins are obtained. For example, when the expression vector includes lac promoter, it is possible to induce gene expression by treating IPTG to the host cells.

Besides, the present disclosure relates to a pharmaceutical composition that comprises a pharmaceutically effective amount of a nucleic acid molecule or a gene carrier including the nucleic acid molecule and a pharmaceutically acceptable carrier. For example, the nucleic acid molecule or the gene vehicle may be used as a genetic vaccine, a gene therapy or an adjuvant. For example, the pharmaceutical composition may comprise the nucleic acid molecule or the gene carrier including the molecule as an adjuvant, a pharmaceutically acceptable carrier, and optionally a pharmaceutically active ingredient. In an exemplary embodiment, the nucleic acid molecule may be administered to a subject directly or as the gene delivery vehicle.

In one embodiment, the pharmaceutical composition as a vaccine may includes a nucleic acid molecule. In this case, the nucleic acid molecule may comprise at least one expression control sequence “MCS”, “MCS1” and “MCS2” including a viral IRES element and at least one coding region “CR”, “CR1” and “CR2” linked operatively to the at least one expression control sequence “MCS”, “MCS1” and “MCS2” and encoding a peptide or a protein, and optionally at least one of multiple adenosines or multiple thymidines upstream of the at least one expression control sequence “MCS”, “MCS1” and “MCS2”.

As an example, the at least one expression control sequence “MCS”, “MCS1” and “MCS2” of the nucleic acid molecule in the pharmaceutical composition as a vaccine may comprise a viral 5′ untranslated region (5′ UTR). In this case, the nucleic acid molecule may further comprise a viral 3′ Untranslated Region (3′ UTR) located downstream of the 5′ UTR, and the at least one coding region “CR”, “CR1” and “CR2” may be located between the 5′ UTR and the 3′ UTR. In one embodiment, the at least one coding region “CR”, “CR1” and “CR2” may encode an antigen or fragments thereof, particularly a peptide or a protein selected from the group consisting of a viral pathogen, a viral antigen and combination thereof. Alternatively, the at least one coding region “CR”, “CR1” and “CR2” may encode a protein or fragments thereof for treating disease.

Alternatively, the at least one expression control sequence “MCS” of the nucleic acid molecule in the pharmaceutical composition as a vaccine may comprise a first expression control sequence “MCS1” having a first IRES element (e.g. 5′ UTR 1) and a second expression control sequence “MCS2” located downstream of the first expression control sequence “MCS2” and having a second IRES element (e.g. 5′ UTR 2). In this case, the at least one coding region (CR) may comprise a first coding region “CR1” located between the first and second expression control sequences “MCS1” and “MCS2” and a second coding region “CR2” located downstream of the second expression control sequence “MCS2”. Besides, the nucleic acid molecule may further comprise at least one of multiple adenosines or multiple thymidines upstream of at least one of the first expression control sequence “MCS1” and the second expression control sequence “MCS2”. As an example, the first expression control sequence “MCS1” may comprises a first viral IRES element derived from coxsackie B virus or Cricket paralysis virus, and the second expression control sequence “MCS2” may comprise a second viral IRES element derived from Encephalomyocarditis virus. If necessary, the nucleic acid molecule may further comprise a transcription control sequence (TCS) located upstream of the at least one expression control sequence “MCS1”, and a polyadenylation signal sequence or a poly adenosine sequence (PA) downstream of the at least one coding region (CR). Preferably, the nucleic acid molecule may have RNA platform.

In another embodiment, the pharmaceutical composition as an adjuvant may includes a nucleic acid molecule. In this case, the nucleic acid molecule may act as an adjuvant. The nucleic acid molecule as an adjuvant may comprise at least one expression control sequence “MCS”, “MCS1” and “MCS2” including a viral IRES element. Optionally, the nucleic acid molecule as an adjuvant may further comprise at least one coding region “CR”, “CR1” and “CR2” linked operatively to the at least one expression control sequence “MCS”, “MCS1” and “MCS2” and encoding a peptide or a protein, and optionally at least one of multiple adenosines or multiple thymidines located upstream of the at least one expression control sequence “MCS”, “MCS1” and “MCS2”.

As an example, the at least one expression control sequence “MCS”, “MCS1” and “MCS2” of the nucleic acid molecule as an adjuvant may comprise a viral 5′ untranslated region (5′ UTR). In this case, the nucleic acid molecule as an adjuvant may further comprise a viral 3′ Untranslated Region (3′ UTR) located downstream of the 5′ UTR, and the at least one coding region “CR”, “CR1” and “CR2” may be located between the 5′ UTR and the 3′ UTR. In one embodiment, the at least one coding region “CR”, “CR1” and “CR2” may encode an antigen or fragments thereof, particularly a peptide or a protein selected from the group consisting of a viral pathogen, a viral antigen and combination thereof. Alternatively, the at least one coding region “CR”, “CR1” and “CR2” may encode a protein or fragments thereof for treating disease.

Alternatively, the at least one expression control sequence “MCS” of the nucleic acid molecule as an adjuvant may comprise a first expression control sequence “MCS1” having a first IRES element (e.g. 5′ UTR 1) and a second expression control sequence “MCS2” located downstream of the first expression control sequence “MCS2” and having a second IRES element (e.g. 5′ UTR 2). In this case, the at least one coding region (CR) may comprise a first coding region “CR1” located between the first and second expression control sequences “MCS1” and “MCS2” and a second coding region “CR2” located downstream of the second expression control sequence “MCS2”. Besides, the nucleic acid molecule as an adjuvant may further comprise at least one of multiple adenosines or multiple thymidines upstream of at least one of the first expression control sequence “MCS1” and the second expression control sequence “MCS2”. As an example, the first expression control sequence “MCS1” may comprises a first viral IRES element derived from coxsackie B virus or Cricket paralysis virus, and the second expression control sequence “MCS2” may comprise a second viral IRES element derived from Encephalomyocarditis virus. If necessary, the nucleic acid molecule as an adjuvant may further comprise a transcription control sequence (TCS) upstream of the at least one expression control sequence “MCS1”, and a polyadenylation signal sequence or a poly adenosine sequence (PA) downstream of the at least one coding region (CR). Preferably, the nucleic acid molecule may have RNA platform.

In one embodiment, when the nucleic acid molecule or the gene delivery vehicle including the molecule is used as vaccine, the pharmaceutical composition may further comprise an adjuvant for enhancing immunogenicity of the vaccine. Such adjuvant may be selected by its immunogenicity and other pharmaceutical properties of the ingredients.

In one exemplary embodiment, the pharmaceutical composition is formulated as liquid, the pharmaceutically acceptable carrier may comprise, but are not limited to, pyrogen-free water; isotonic saline or buffered (water) solution such as phosphate or citrate; plant oil such as peanut oil, cotton seed oil, sesame oil, olive oil, corn oil and cacao fruit oil; glycols such as propylene glycol, glycerol, sorbitol, mannitol and polyethylene glycol; and polyol such as alginic acid. In this case, aqueous buffer including sodium salts, calcium salts, and optionally potassium salts can be used for injecting liquid pharmaceutical composition into bodies. Sodium salts, calcium salts and potassium salts may have halogenized type such as iodine or bromine, hydroxide, carbonate salt, hydrogen carbonate salt or sulfonate salts.

When the pharmaceutical composition is formulated as solid, the pharmaceutically acceptable carrier may comprise solid carrier such as solid filter, liquid filter or diluents, and encapsulating compound may be used as the carrier for administering the composition. For example, the pharmaceutically acceptable carrier may comprise, but are not limited to, sugar such as lactose, glucose and sucrose; starch such as corn starch of potato starch; cellulose or its derivative such as sodium carboxylmethyl cellulose, ethyl cellulose and cellulose acetate; powdered tragacanth; malt; gelatins; tallow; solid lubricant such as stearic acid and magnesium stearate; and calcium sulfate.

The pharmaceutically acceptable carrier may comprise hydrogel, adjusted release devices, delayed release devices, polylactic acid and collagen matrix for injection. The pharmaceutically acceptable carrier appropriate for local uses may comprises lotion, cream, gel and similar thereof. If the composition is orally administered, tablet, capsule is preferred unit dosage form.

The pharmaceutically acceptable carrier may be selected as the administering types of the composition. In one embodiment, the composition may be administered systemically. The administering route may comprise in oral, intracutaneous, intravenous, intra muscular, intra-articular, intrsynovial, intrathecal, intrhepatic, intralesional, intracranial, transdermal, intradermal, intrapumonal, intraperitoneal, intracardial, intraarterial, sublingual topical and/or intranasal.

The pharmaceutical composition may be administered with any convenient type, for example, tablet, powder, capsule, solution, dispersion, suspension, syrup, spray, suppository, gel, emulsion, and patch. The pharmaceutical composition may further common additives such as buffer agent, stabilizing agent, surfactant, wetting agent, lubricant, emulsifier, suspendered agent, conservative, anti-oxidant, opacifying agent, slip modifier, processing aids, coloring agent, sweetener, perfume, flavoring agent, diluents and other additives. Besides, the pharmaceutical composition may comprise any enhancing agent such as cytotoxic agent, cytokine, chemo-therapeutics, growth-inhibitor or growth-enhancer. For example, the pharmaceutical composition may contain emulsifier such as Tween; wetting agent such as sodium lauryl sulfate; coloring agent; taste-imparting agent; tablet-forming agents, stabilizing agent; anti-oxidant; and conservatives.

As described above, the pharmaceutical composition may comprise the gene delivery vehicle. The gene delivery vehicle is fabricated in order to transfer and express the nucleic acid molecule. In one embodiment, the transcript of GOI may be within an appropriate expression construct so as to fabricate the gene delivery vehicle and may be linked operatively to the transcription control sequence, e.g. promoter within the expression construct. The promoter linked operatively to GOI may act within animal cells, preferably mammalian cells so as to regulate the transcription of the nucleic acid molecule, and may comprise mammalian-virus derived promoters, mammalian-genome derived promoters and bacteriophage derived promoters. For example, the promoter may comprise, but are not limited to, Cytomegalovirus (CMV) promoter, adenovirus late promoter, vaccinia virus 7.5K promoter, SV40 promoter, tK promoter of HSV, T7 promoter, T3 promoter, SM6 promoter, RSV promoter, EF1 alpha promoter, metallothionein promoter, beta-action promoter, human IL-2 gene promoter, human IFN gene promoter, human IL-4 gene promoter, human lymphotoxin gene promoter, human GM-CSF gene promoter, tumor-cell specific promoter (e.g. TERT promoter, PSA promoter, PSMA promoter, CEA promoter, E2F promoter and AFT promoter) and tissue-specific promoter (e.g. albumin promoter). Beside, the expression construct may comprise polyadenylation signal sequence (e.g. bovine growth hormone terminator and/or SV40-derived polyadenylation signal sequence).

An appropriated transcription control sequence (TCS) enabling IVT may be located upstream of the expression control sequence (ECS), “ECS1” and (ECS) in case the nucleic acid molecules are utilized as RAN vaccine. Since the nucleic acid molecules of RNA platform can be utilized as RNA vaccine and can be synthesized IVT process, it is not necessary to treat living viruses or pathogenic bacteria used in fabricating general live vaccine or killed vaccine and to culture host cells such as yeast, E. coli and/or insect cells to express the recombinant peptides or proteins.

For example, the nucleic acid molecules of DNA platform are inserted into plasmid and then are transcribed into mRNA via IVT process in which the mRNA is synthesized in vitro by RNA polymerase using a linear DNA with cutting ends by restriction endonuclease so as to produce RNA vaccine using the nucleic acid molecules. The transcription control sequence (TCS) derived from bacteriophage may be located upstream of the expression control sequence (ECS) for transcribing linearized DNA to RNA. For example, the transcription signal sequence (TCS) may be any promoters can transcribe linearized DNA into mRNA and may comprise, but are not limited to, T7 bacteriophage promoter, T3 bacteriophage promoter and SP6 bacteriophage promoter. In one exemplary embodiment, the transcription control sequence (TCS) may be located adjacently to, preferably upstream of the expression control sequence (ECS).

The gene delivery vehicle may be fabricated with various forms, for example, plasmid, viral vector, and/or liposomes or niosome including the plasmid. In one embodiment, the transcript of GOI may be applied into any gene delivery system, for example, plasmid, adenovirus (Lockett L. J., et al., Clin. Cancer Res. 3:2075-2080, 1997), adeno-associated virus (AAV; Lashford L. S., et al., Gene Therapy Technologies, Applications and Regulations Ed. A. Meager, 1999), retrovirus (Gunzburg W. H., et al., Retroviral vectors. Gene Therapy Technologies, Applications and Regulations Ed. A. Meager, 1999), lentivirus (Wang G. et al., J. Clin. Invest. 104(11): R55-62, 1999), herpes simplex virus (Chamber R., et al., Proc. Natl. Acad. Sci USA 92:1411-1415, 1995), vaccinia virus (Puhlmann M. et al., Human Gene Therapy 10:649-657, 1999), liposome (Methods in Molecular Biology, Vol 199, S. C. Basu and M. Basu (Eds.), Human Press, 2002) and/or niosome.

Beside, the gene delivery vehicle may be transfected into host cells by known various methods. In one embodiment, the gene delivery vehicle may be transfected in accordance with kwon viral infection methods in case it is fabricated based upon viral vectors. The infection of host cells using the viral vector was described in the above literatures, each of which is incorporated herein by reference with its entirety.

For example, the gene delivery system comprises a naked DNA molecule or plasmid, it can be transfected into the host cells using anyone of microinjection method (Capecchi, M. R., Cell, 22:479, 1980; Harland and Weintraub, J. Cell Biol. 101:1094-1099, 1985), calcium-phosphate precipitate method (Graham, F. L. et al., Virology, 52:456, 1973; and Chen [symbol] and Okayama, Mol. Cell. Biol. 7:2745-2752, 1987), electroporation method (Neumann, E. et al., EMBO J., 1:841, 1982; and Tur-Kaspa et al., Mol. Cell Biol., 6:716-718 (1986)), liposome-mediated transfection method (Wong, T. K. et al., Gene, 10:87, 1980; Nicolau and Sene, Biochim. Biophys. Acta, 721:185-190, 1982; and Nicolau et al., Methods Enzymol., 149:157-176, 1987), DEAE-dextran treating method (Gopal, Mol. Cell Biol., 5:1188-1190, 1985), and gene bombardment (Yang et al., Proc. Natl. Acad. Sci., 87:9568-9572, 1990), each of which is incorporated herein by reference with its entirety.

In an exemplary embodiment, the nucleic acid molecule and/or the gene delivery vehicle including the molecule may be used as an adjuvant. For example, the nucleic acid molecule may be stabilized in the pharmaceutical composition using cationic polymers, cationic peptides or cationic polypeptides. The cationic (poly) peptides as a stabilizing agent may be comprise multiple cationic polymers such as poly-lysine and poly-arginine, cationic lipids or lipofectants. More concretely, the stabilizing agent may comprise, but are not limited to, a histone, a nucleoline, protamine, oligofectamine, spermine or spermidine, and cationic polysaccharides, in particular chitosan, TDM, MDP, muramyl dipeptide, pluronics, and/or derivatives thereof. Histones and protamines are cationic proteins which naturally compact DNA. As histones which may be used in the context of the present disclosure to form a complex with the nucleic acid molecule as the adjuvant may be made of histones H1, H2a, H3 and H4. Also, as protamines which may be used in the context of the present disclosure to form a complex with the nucleic acid molecule may be made of protamin P1 or P2 or cationic partial sequences of protamine. If necessary, other compounds that can form a complex with the nucleic acid molecule may be another adjuvant additionally used.

The nucleic acid molecule as an adjuvant may induce a non-antigen specific immune responses. T lymphocytes is differentiated into T-helper 1 (Th1) cells and T-helper 2 (Th2) cells and immune system can destroy intra-cellular pathogens (e.g. antigens) by Th1 cells and extra-cellular pathogens by Th2 cells. Th1 cells helps cell-mediated immune response by activating macrophages and cytotoxic T-cells, while Th2 cells facilitates humoral immune responses by enhancing B-cell for transformation into cytoplasmic cells and by forming antibodies against the antigens. Accordingly, the ratio of Th1 cells/Th2 cells in immune response is very significant. The nucleic acid molecule can enhance and induce Th1 immune response, i.e. cell-mediated immune responses. In one exemplary embodiment, when the nucleic acid molecule is injected into body together with a pharmaceutically active ingredient, e.g. immunity enhancing components, the nucleic acid molecule may act as adjuvant that enhancing specific immune responses induced by the pharmaceutically active ingredients.

Accordingly, the pharmaceutical composition may comprise a pharmaceutically active ingredient as well as the nucleic acid molecule as the adjuvant. In one exemplary embodiment, the pharmaceutically active ingredient may be an immunity enhancer such as an immunogen. For example, the pharmaceutically active ingredient may comprise a compound treating and/or preventing cancers, infectious diseases, autoimmune diseases and/or allergies. In one embodiment, the pharmaceutically active ingredient may comprise, but are not limited to, peptides, proteins, nucleic acids, therapeutically active low-molecular organic or inorganic compounds, sugars, antigens or antibodies, therapeutics known to the art, antigen cells, fragments of antigen cells, cell debris, pathogens (including viruses or bacteria) modified chemically or light irradiations such as attenuated or inactivated pathogens. For example, the antigens as the pharmaceutically active ingredient may be peptides, polypeptides, proteins, cells, cell extracts, polysaccharides, complex polysaccharides, lipids, glycolipids and carbohydrates. The antigens as the pharmaceutically active ingredients may comprise, but are not limited to, tumor antigens, animal antigens, vegetation antigens, viral antigens, bacterial antigens, fungal antigens, protozoan antigens, autoimmune antigens and/or allergic antigens each of which may be expressed from the coding region “CR”, “CR1” and “CR2”.

For example, the antigens may have secreted forms of surface antigens of tumor cells, viral pathogens, bacterial pathogens, fungal pathogens and/or protozoan pathogens. If necessary, the antigens may be in the nucleic acid molecule according to the present disclosure, or as heptene bound to an appropriate carrier. Other antigenic components, for example, inactivated or attenuated pathogens may be used.

In another exemplary embodiment, antibodies, preferably therapeutically effective antigens may be used as the pharmaceutically active ingredient. For examples, antibodies against the cancers or infectious diseases such as cell-surface proteins, peptides or proteins expressed from tumor-suppressor genes or inhibitor genes, growth factors or elongation factors, apoptosis-associated proteins, tumor antigens, and above-mentioned antigens, proteins or nucleic acids may be preferably used as the pharmaceutically active ingredient. Such antigens and/or antibodies are described above. For example, the pharmaceutical composition may be utilized as a vaccine in case of using the antigens as the pharmaceutically active ingredient or as disease therapeutics in case of using the antibodies as the pharmaceutically active ingredient.

In one exemplary embodiment, when the coding region “CR”, “CR1” and “CR2” encodes antigens, antibodies and fragments thereof in the nucleic acid molecules used as the adjuvant, the pharmaceutically active ingredient may be the antigens, antibodies and fragments thereof. For example, the pharmaceutically active ingredient may comprise, but are not limited to, spike peptide of MERS-CoV (SEQ ID NO: 20), L1 region of HPV, for example, L1 region of HPVs such as HPV-6, HPV-11, HPV-16, f HPV-18, HPV-31, HPV-33, HPV-35, HPV-45, HPV-52 and/or HPV-58, surface antigens of influenza virus such as Haemagglutin, for example iPR8 (influenza A/Puerto Rico/08/34; SEQ ID NO: 24), fragments thereof and equivalents thereof.

An amount of the pharmaceutical composition may be determined by common experiments using animal models. Such an animal model may comprise, but are not limited to, rabbit, sheep, mouse, dog and non-human primates.

If necessary, the pharmaceutical composition may further at least one auxiliary substances so as to further increase immunogenicity induced by the pharmaceutically active ingredient and/or the nucleic acid molecule as the adjuvant. For example, substances that allow maturation of dendritic cells (DCs), for example, lipopolysaccharides, TNF-alpha or CD40 ligand, form such auxiliary substances. In general, it is possible to use as auxiliary substance any agent that influences the immune system in the manner of a “danger signal” (LPS, GP96, and the likes) or cytokines, such as GM-CFS, which allow an immune response produced by the nucleic acid molecules.

If necessary, the pharmaceutical composition may further additional adjuvant as well as the nucleic acid molecule as the main adjuvant. The additional adjuvant enhances immunological activities of the pharmaceutically active ingredient and/or the nucleic acid molecule. In this case, the nucleic acid molecule combined with additional adjuvants. Suitable agents or adjuvants for these purposes are in particular those compounds that enhance (by one or more mechanisms) the biological property/properties of the nucleic acid molecule.

Particularly preferred as cationic or polycationic compounds are compounds selected from the group consisting of protamine, nucleoline, spermin, spermidine, oligoarginines as defined above, such as Arg7, Arg8, Arg9, Arg7, H5R9, R9H5, H5R9H5, YSSR9SSY, (RKH)4, Y(RKH)2R, and the likes.

Besides, any compound, which is known to be immune-stimulating due to its binding affinity (as ligands) to Toll-like receptors: TLR1, TLR2, TLR3, TLR4, TLR5, TLR6, TLR7, TLR8, TLR9, TLR10, TLR11, TLR12 or TLR13 may suitably be used as further component to further stimulate the immune response induced by nucleic acids of the invention in the inventive pharmaceutical compositions.

Another class of compounds, which may be added to the pharmaceutical composition of the disclosure, are CpG nucleic acids, in particular CpG-RNA or CpG-DNA. A CpG-RNA or CpG-DNA can be a single-stranded CpG-DNA (ss CpG-DNA), a double-stranded CpG-DNA (dsDNA), a single-stranded CpG-RNA (ss CpG-RNA) or a double-stranded CpG-RNA (ds CpG-RNA). The CpG nucleic acid is preferably in the form of CpG-RNA, more preferably in the form of single-stranded CpG-RNA (ss CpG-RNA). The CpG nucleic acid preferably contains at least one or more (mitogenic) cytidine (cytosine)/guanine dinucleotide sequence(s) (CpG motif(s)). According to a first preferred alternative, at least one CpG motif contained in these sequences, that is to say the C (cytidine (cytosine)) and the G (guanine) of the CpG motif, is unmethylated. All further cytidines (cytosines) or guanines optionally contained in these sequences can be either methylated or unmethylated. According to a further preferred alternative, however, the C (cytidine (cytosine)) and the G (guanine) of the CpG motif can also be present in methylated form.

When the nucleic acid molecule is mixed with another adjuvant, the mixing ratio is not specifically limited. For example, the nucleic acid molecule may be mixed with the additional adjuvant with a ratio of about 100:1 to about 1:100, preferably about 10:1 to about 1:10, more preferably about 5:1 to about 1:5, most preferably about 3:1 to 1:3 by weight. Besides, the contents or the concentration of the nucleic acid molecule as the adjuvant is not specifically limited. Particularly, when the nucleic acid molecule of RNA platform is used, it can be degraded rapidly in bodies so as to obtain improved safety and stability. In one exemplary embodiment, the nucleic acid molecule may be contained with a concentration of about 1 to about 1000 μg/mL, preferably about 10 to about 1000 μg/mL within the pharmaceutical composition.

In another embodiment, the pharmaceutical composition may be provided as vaccines. The vaccine composition may comprise immune-enhancing substances as the pharmaceutically active ingredient that induces adjusted immune response against specific antigen. Such adjusted immune response causes an individual to develop adaptive immune response induced by active or passive mode against specific pathogen or specific tumor.

The vaccines as the pharmaceutical composition may be used for treating following diseases and/or disorders. The pharmaceutical composition as the vaccine can be utilized as treating and/or preventing tumor-specific diseases or pathogen-specific diseases, infection diseases, allergic diseases and/or auto-immune diseases or disorders.

An important factor for a suitable immune response is the stimulation of different T-cell sub-populations. T-lymphocytes typically differentiate into two sub-populations, the T-helper 1 (Th1) cells and the T-helper 2 (Th2) cells, with which the immune system is capable of destroying intracellular (Th1) and extracellular (Th2) pathogens (e.g. antigens). The two T-helper (Th) cell populations differ in the pattern of effector proteins (cytokines) produced by them. Thus, Th1 cells assist the cellular immune response by activation of macrophages and cytotoxic T-cells. On the other hand, Th2 cells promote the humoral immune response by stimulation of B-cells for conversion into plasma cells and by formation of antibodies (e.g. against antigens). The Th1/Th2 ratio is therefore of great importance in the immune response. In an exemplary embodiment, the nucleic acid molecule can stimulate or enhance Th1 immune response.

In one exemplary embodiment, the pharmaceutical composition may be used for inducing tumor-specific or pathogen-specific immune response.

In another exemplary embodiment, the pharmaceutical composition including the nucleic acid molecule can be used for the treatment of infectious diseases, but are not limited to, such as influenza, malaria, SARS, yellow fever, AIDS, and the likes.

In still another exemplary embodiment, the pharmaceutical composition can be used for the preparation of a medicament for the treatment of an allergic disorder or disease. Allergy is a condition that typically involves an abnormal, acquired immunological hypersensitivity to certain foreign antigens or allergens. Allergies normally result in a local or systemic inflammatory response to these antigens or allergens and leading to immunity in the body against these allergens. Allergens in this context include e.g. grasses, pollens, molds, drugs, or numerous environmental triggers, and the likes Without being bound to theory, several different disease mechanisms are supposed to be involved in the development of allergies. According to a classification scheme by P. Gell and R. Coombs the word “allergy” was restricted to type I hypersensitivities, which are caused by the classical IgE mechanism. Type I hypersensitivity is characterized by excessive activation of mast cells and basophils by IgE, resulting in a systemic inflammatory response that can result in symptoms as benign as a runny nose, to life-threatening anaphylactic shock and death. Well known types of allergies include, without being limited thereto, allergic asthma (leading to swelling of the nasal mucosa), allergic conjunctivitis (leading to redness and itching of the conjunctiva), allergic rhinitis (“hay fever”), anaphylaxis, angiodema, atopic dermatitis (eczema), urticaria (hives), eosinophilia, respiratory, allergies to insect stings, skin allergies (leading to and including various rashes, such as eczema, hives (urticaria) and (contact) dermatitis), food allergies, allergies to medicine, and the likes.

For example, the pharmaceutical composition of the present disclosure may treat and/or prevent allergic disorders or diseases derived from an allergen (e.g. from a cat allergen, a dust allergen, a mite antigen, a plant antigen (e.g. a birch antigen) and the likes) either as a protein. The pharmaceutical composition may shift the exceeding immune response to a stronger TH1 response, thereby suppressing or attenuating the undesired IgE response.

In still another embodiment, the pharmaceutical composition may be used for the preparation of a medicament for the treatment of autoimmune diseases. Autoimmune diseases can be broadly divided into systemic and organ-specific or localized autoimmune disorders, depending on the principal clinico-pathologic features of each disease. Autoimmune disease, can be treated or prevented by the pharmaceutical composition, may be divided into the categories of systemic syndromes, including SLE, Sjφgren's syndrome, Scleroderma, Rheumatoid Arthritis and polymyositis or local syndromes which may be endocrinologic (DM Type 1, Hashimoto's thyroiditis, Addison's disease and the likes), dermatologic (pemphigus vulgaris), haematologic (autoimmune haemolytic anaemia), neural (multiple sclerosis) or can involve virtually any circumscribed mass of body tissue. The autoimmune diseases to be treated may be selected from the group consisting of type I autoimmune diseases or type II autoimmune diseases or type III autoimmune diseases or type IV autoimmune diseases, such as, for example, multiple sclerosis (MS), rheumatoid arthritis, diabetes, type I diabetes (Diabetes mellitus), systemic lupus erythematosus (SLE), chronic polyarthritis, Basedow's disease, autoimmune forms of chronic hepatitis, colitis ulcerosa, type I allergy diseases, type II allergy diseases, type III allergy diseases, type IV allergy diseases, fibromyalgia, hair loss, Bechterew's disease, Crohn's disease, Myasthenia gravis, neurodermitis, Polymyalgia rheumatica, progressive systemic sclerosis (PSS), psoriasis, Reiter's syndrome, rheumatic arthritis, psoriasis, vasculitis, etc, or type II diabetes.

In accordance with another aspect, the present disclosure relates to a method for stimulating, enhancing or inducing an immune response, comprising administering therapeutically effective amount of the nucleic acid molecule and/or the gene delivery vehicle including the nucleic acid molecule to a subject. If necessary, the nucleic acid molecule and/or the gene delivery vehicle may be administered together with a pharmaceutically acceptable carrier and/or additional additives used commonly in a pharmaceutical or medicinal field.

### Example 1: Fabrication of Nucleic Acid Molecule of RNA Platform

An artificial nucleic acid molecule of RNA platform including a viral IRES element derived from Sindbis virus (SV) was fabricated. A template DNA having the following ordered sequence was designed:

5′-KpnI recognition site (GGTACC)—T7 promoter (SEQ ID NO: 14)—SV 5′ UTR (SEQ ID NO: 1) as IRES element—DLP structure in NSP1 (SEQ ID NO: 11)—BamHI recognition site and Kozak sequence (GGATCC GACC) (SEQ ID NO: 30)—Renilla luciferase (R/L) as ORF (SEQ ID NO: 16)—EcoRV-SacI-EcoRI recognition sites (GATATC GCGAGC GAATTC)(SEQ ID NO: 40)—SV 3′ UTR (SEQ ID NO: 7)—poly A 50—NotI recognition site (GCGGCCGC)—3′.

The Renilla luciferase coding sequence was amplified using forward and reverse primers that covered the restriction site for the insertion of the MCS into each RNA platform. The GOIs were inserted into the MCS of the RNA platform using restriction endonucleases (New England Biolabs, USA). Escherichia coli DH5α-competent cells were used for plasmid preparation, and all plasmid clones were checked by restriction mapping and direct DNA sequencing (Cosmo Genetech, Korea). Transfection-grade plasmid was obtained using LaboPass Plasmid Mini Purification Kits, according to the manufacturer's instructions (Cosmo Genetech). The template DNA was cloned into pGH vector and linearized using restriction endonuclease. In vitro transcription (IVT) was performed using the Ribomax Large-scale RNA Production System T7 (Promega, USA). For in vitro transcription, all platforms were linearized with NotI. Transcription reactions contained 3 μg of Not I-cut plasmid DNA, T7 transcription buffer (5×), 25 mM rNTP, nuclease-free water, and T7 enzyme mix and were incubated for 4 h at 37° C. The transcripts were incubated with 1 μl of RNase-free DNase I (Promega) per 1 μg of plasmid DNA for 15 min at 37° C., followed by termination of the reaction by incubation at 65° C. for 10 min. DNase I treatment (Promega) was always performed to remove any DNA contamination during RNA purification using highyield RNA ultra-purification kits (RBC, Taiwan), according to the manufacturer's instructions. DNA and RNA purity and concentration were evaluated using a NanoDrop-2000 spectrophotometer (Thermo Fisher-Scientific, USA). The nucleic acid molecule fabricated in this Example will be referred as “pnon-SV-R/L”.

### Example 2: Fabrication of Nucleic Acid Molecule of RNA Platform

An artificial nucleic acid molecule of RNA platform including a viral IRES element derived from coxsackie B virus (CVB3) was fabricated by repeating the same process as Example 1 except undergoing ARCA reaction and using the following ordered template DNA:

5′-BamHI recognition site (GGATCC)—T7 promoter (SEQ ID NO: 14)—CVB3 5′ UTR (SEQ ID NO: 2) as IRES element—expression enhancer sequence (ATGGCAGCTCAA) (SEQ ID NO: 29)—SalI recognition site and Kozak sequence (GTCGAC GACC) (SEQ ID NO: 30)—R/L as ORF (SEQ ID NO: 16)—SacII-PvuI recognition sites (CCGCGG CGATCG) (SEQ ID NO: 31)—CVB3 3′ UTR (SEQ ID NO: 8)—poly A 50—NotI recognition site (GCGGCCGC)—3′.

The template DNA was treated with ARCA reaction for capping at the 5′ ends of RNA sequences after in vitro transcription. For capped transcript, 40 mM 3′-O-Me-m7G, (5′)ppp(5′)G ACRA was included, and the concentration of rGTP was decreased to 3 mM. The nucleic acid molecule fabricated in this Example will be referred as “pCAP-CVB3-R/L”.

### Example 3: Fabrication of Nucleic Acid Molecule of RNA Platform

An artificial nucleic acid molecule of RNA platform including a viral IRES element derived from Encephalomyocarditis virus (EMCV) was fabricated by repeating the same process as Example 1 except using the following ordered template DNA:

5′-EcoRI recognition site (GAATTC)—T7 promoter (SEQ ID NO: 14)—EMCV 5′ UTR (SEQ ID NO: 3) as IRES element—BamHI recognition site and Kozak sequence (GGATCC GACC)(SEQ ID NO: 30)—R/L as ORF (SEQ ID NO: 16)—SacII-PvuI recognition sites (CCGCGG CGATCG)(SEQ ID NO: 31)—EMCV 3′ UTR (SEQ ID NO: 9)—poly A 50—NotI recognition site (GCGGCCGC)—3′. The nucleic acid molecule fabricated in this Example will be referred as “pEMCV-R/L”

### Example 4: Fabrication of Nucleic Acid Molecule of RNA platform

Artificial nucleic acid molecule of RNA platform including a viral IRES element derived from Japanese encephalitis virus (JEV) was fabricated by repeating the same process as Example 1 except undergoing ARCA reaction and using the following ordered template DNA:

5′-KpnI recognition site (GGTACC)—T7 promoter (SEQ ID NO: 14)—JEV 5′ UTR (SEQ ID NO: 5) as IRES element—partial JEV core (SEQ ID NO: 13)—BamHI recognition site and Kozak sequence (GGATCC GACC) (SEQ ID NO: 30)—R/L as ORF (SEQ ID NO: 16)—SacII-PvuI recognition sites (CCGCGG CGATCG) (SEQ ID NO: 31)—JEV 3′ UTR (SEQ ID NO: 10)—poly A 50—NotI recognition site (GCGGCCGC)—3′. The template DNA was treated with ARCA reaction as Example 2. The nucleic acid molecule fabricated in this example will be referred as “pCAP-JEV-R/L”

### Example 5: Fabrication of Nucleic Acid Molecule of RNA Platform

An artificial nucleic acid molecule of RNA platform using IRES element derived from cricket paralysis virus (CrPV) was fabricated by repeating the same process as Example 1 except using the following ordered template DNA:

5′-KpnI recognition site (GGTATC)—T7 promoter (SEQ ID NO: 14)—CrPV IGR IRES (SEQ ID NO: 6) as IRES element—start codons (CCT GCT)—R/L as ORF (SEQ ID NO: 16)—SV40 late polyadenylation signal sequence (SEQ ID NO: 15)—NotI recognition site (GCGGCCGC)—3′. The nucleic acid molecule fabricated in this example will be referred as “pCrPV-R/L”

### Comparative Example 1: Fabrication of Nucleic Acid Molecule of RNA Platform

An artificial nucleic acid molecule of cap dependent RNA platform including eukaryotic UTRs derived from human ribosomal proteins, in accordance with WO 2015/101414 (assigned to CureVac AG) was fabricating by repeating the same process as Example 1 except undergoing ACRC reaction and using the following ordered template DNA:

5′-KpnI recognition site (GGTACC)—T7 promoter (SEQ ID NO: 14)—Human ribosomal protein large 32 5′ UTR (SEQ ID NO: 26)—linker sequence ((AAGCTTGAGG)(SEQ ID NO: 32)—BamHI recognition site and Kozak sequence (GGATCC GACC) (SEQ ID NO: 30)—R/L as ORF (SEQ ID NO: 16)—EcoRI recognition site (GAATTC)—linker sequence (GACTAGT)—Human ribosomal protein small 9 3′ UTR (SEQ ID NO: 27)—linker sequence (AGATCT)—poly A 64—linker sequence (ATGCATC)—Histone stem-loop sequence (SEQ ID NO: 28)—NotI recognition site (GCGGCCGC)—3′. The template DNA was treated with ARCA reaction as Example 2. The nucleic acid molecule fabricated in this example will be referred as “pCAP-curevac-R/L”

### Experimental Example 1: Measurement of Expression Efficiency of Nucleic Acid Molecule

Each of nucleic acid molecules in Examples 1-5 (pnon-SV-R/L, pCAP-CVB3-R/L, pEMCV-R/L, pCAP-JEV-R/L and pCrPV-R/L) and Comparative Example 1 (pCAP-curevac-R/L) of RNA platform was transfected into human muscle cells A204 and human embryonic kidney cells 293T. Human A204 rhabdomyosarcoma cells and human 293T embryonic kidney cells were obtained from the Korean Cell Line Bank (Korea). A204 cells were maintained in McCoy's 5A medium (Gibco, Thermo Fisher Scientific) supplemented with 10% fetal bovine serum (FBS; Gibco) and 1% antibiotics (AAs, Gibco). 293T cells were maintained in Dulbecco's Modified Eagle's Medium (Hyclone, GE Healthcare, UK) containing 10% FBS and 1% amino acids. All cells were maintained in a humidified atmosphere at 37° C. with 5% CO2. For luciferase assays, aliquots of 2×105 cells were seeded in 48-well plates and cultured at 37° C. for 24 hours, followed by replacement of the medium with medium lacking FBS before transfection. Cells were transfected at 80%-90% confluence using lipofectamine 2000 (Invitrogen, Thermo Fisher Scientific) transfection reagent, according to the manufacturer's instructions. In the assays, 500 ng of RNA (R/L), 5 ng of plasmid DNA (F/L, from Prof. Yoon H W, Medical Center, Seoul University), and 1.25 μg of lipofectamine were mixed with 50 μL of Opti-MEM medium (Gibco) per well and incubated at room temperature for 5 minutes. Subsequently, diluted RNA and DNA were mixed with diluted lipofectamine, incubated at room temperature for 15 min, and co-transfected into a prepared 48-well plate. In addition, 1 μg of a control green fluorescent protein plasmid was introduced, to normalize transfection efficiency. Cells were harvested 24 hours after transfection, and luciferase assays were carried out using a dual-luciferase assay (Promega). All reagents were prepared as described by the manufacturer. The 5× passive lysis buffer (PLB) was supplied by the manufacturer and used for cell lysis. Briefly, cells were resuspended in 80 μL/well of 1×PLB. After allowing lysis for 15 minutes, 20 μL of each lysate was transferred to a 96-well white assay plate (Corning Costar Corp., USA) and measurements were performed using a Glomax Discover system (Promega). An aliquot of 100 μL of firefly luciferase (F/L) reagent (LAR II) was added to the test sample and luminescence was measured; this was followed by the addition of 100 μL of Renilla luciferase reagent and firefly luciferase quenching reagent (Stop & Glo; Promega), to measure luminescence with an integration time of 10 seconds. The data are reported as the ratio of Firefly to Renilla luciferase activity. This ratio was generated from the results of the control RNA (pCAP-curevac-R/L) divided by those obtained from other RNA platforms. Results are presented as the mean relative light units and the standard deviations (SDs) of at least three independent repeats. To assess the statistical significance of differences in luciferase activity and mRNA expression levels between the various treatment groups, the results were analyzed using the Kruskal-Wallis test, followed by the Bonferroni post-hoc test for comparing multiple groups. Two-tailed p-values <0.05 were considered statistically significant. Data are expressed as the mean±SD. Statistical analysis of the data was performed using SAS software (v, 9.4; SAS Institute, Cary, USA). FIGS. 3A and 3B illustrate expression levels of R/L in A204 cells and in 293T cells. As illustrated in FIGS. 3A and 3B, pEMCV-R/L has a better expression efficiency that did the cap-dependent expression platform pCAP-curevac-R/L in both A204 and 293T cells. Also, pCrPV-R/L showed slightly lower or similar expression pattern compared with pCAP-curevac-R/L. Taken together, RNA expression platforms controlled by viral IRES elements, especially those from EMCV 5′ UTR and CrPV 5′ IGR, are not inferior to cap-dependent RNA expression platforms, at least when compared to the commercially developed pCAP-curevac-R/L expression system.

### Example 6: Fabrication of Nucleic Acid Molecule of RNA Platform

An artificial nucleic acid molecule of RNA platform including a viral IRES element derived from CVB3 was fabricated by repeating the same process and using the same template DNA as Example 2 except ARCA reaction was not performed. The nucleic acid molecule fabricated in this Example will be referred as “pCVB3-R/L”.

### Example 7: Fabrication of Nucleic Acid Molecule of RNA Platform

An artificial nucleic acid molecule of RNA platform including a viral IRES element derived from CVB3 was fabricated by repeating the same process as Example 6 except using a template DNA including multiple adenosines (MA-50) inserted between T7 promoter and CVB3 5′ UTR. The template DNA has the following ordered nucleotides:

5′-BamHI recognition site (GGATCC)—T7 promoter (SEQ ID NO: 14)—multiple adenosines (MA-50)—CVB3 5′ UTR (SEQ ID NO: 2) as IRES element—expression enhancer sequence (ATGGCAGCTCAA (SEQ ID NO: 29)—EcoRI recognition site and Kozak sequence (GAATTC GACC)(SEQ ID NO: 33)—R/L as ORF (SEQ ID NO: 16)—SacII recognition site (CCGCGG)—CVB3 3′ UTR (SEQ ID NO: 8)—poly A 50—NotI recognition site (GCGGCCGC)—3′. The nucleic acid molecule fabricated in this Example will be referred as “pMA-CVB3-R/L”.

### Experimental Example 2: Measurement of Expression Efficiency of Nucleic Acid Molecule

Each of nucleic acid molecules in Examples 2 (pCAP-CVB3-R/L), Example 6 (pCVB3-R/L), Example 7 (pMA-CVB3-R/L) and Comparative Example 1 (pCAP-curevac-R/L) was transfected into human muscle cells A204. Expression levels of Renilla luciferase (R/L) in the cell line were measured as the same process as Experimental Example 1. FIG. 4 illustrates expression levels of R/L in A204 cells. As illustrated in FIG. 4, the addition of multiple adenosines at the 5′ end of the CVB3 IRES (pMA-CVB3-R/L) increased the efficiency of IRES-dependent translation compared with the CVB3 IRES without multiple adenosines (pCVB3-R/L). Moreover, the addition of multiple adenosines at the 5′ end of the CVB3 IRES (pMA-CVB3-R/L) led to better expression levels compared with the cap-binding CVB3 IRES (pCAP-CVB3-R/L).

### Example 8: Fabrication of Nucleic Acid Molecule of RNA Platform

An artificial nucleic acid molecule of RAN platform including multiple viral IRES elements derived from CVB3 and EMCV by repeating the same process as Example 1 except using the following ordered template DNA:

5′-BamHI recognition site (GGATCC)—T7 promoter (SEQ ID NO: 14)—HpaI recognition site (GTTAAC)—multiple adenosines (MA-50)—HpaI recognition site (GTTAAC)—CVB3 5′ UTR (SEQ ID NO: 2) as a first IRES element—expression enhancer sequence (ATGGCAGCTCAA) (SEQ ID NO: 29)—R/L as a first ORF (SEQ ID NO: 17)—EMCV 5′ UTR (SEQ ID NO: 4) as a second IRES element—R/L as a second ORF (SEQ ID NO: 16)—CVB3 3′ UTR (SEQ ID NO: 8)—poly A 50—NotI recognition site (GCGGCCGC)—3′. The nucleic acid molecule fabricated in this Example will be referred as “pMA-CVB3-R/L-EMCV-R/L”.

### Example 9: Fabrication of Nucleic Acid Molecule of RNA Platform

An artificial nucleic acid molecule of RNA platform including multiple viral IRES elements derived from CrPV and EMCV by repeating the same process as Example 1 except using the following template DNA:

5′-BamHI recognition site (GGATCC)—T7 promoter (SEQ ID NO: 14)—PMeI recognition site (GTTTAAAC)—CrPV IGR IRES (SEQ ID NO: 6) as a first IRES element—Start codon (CCT GCT)—EcoRI recognition site (GAATTC)—R/L as a first ORF (SEQ ID NO: 17)—SacI recognition site (GAGCTC)—EMCV 5′ UTR (SEQ ID NO: 4) as a second IRES element—EcoRV-SalI-PacI recognition sites and Kozak sequences (GATATC GTCGAC TTAATTAA GACC)(SEQ ID NO: 34)—R/L as a second ORF (SEQ ID NO: 16)—SacII recognition site (CCGCGG)—SV40 late polyadenylation signal sequence (SEQ ID NO: 15)—NotI recognition site (GCGGCCGC)—3′. The nucleic acid molecule fabricated in this example will be referred as “pCrPV-R/L-EMCV-R/L”.

### Example 10: Fabrication of Nucleic Acid Molecule of RNA Platform

An artificial nucleic acid molecule of RNA platform including multiple viral IRES elements derived from CVB3 and EMCV by repeating the same process as Example 8 except using firefly luciferease ORF (F/L) as the second ORF. The template DNA has the following ordered nucleotides:

5′-BamHI recognition site (GGATCC)—T7 promoter (SEQ ID NO: 14)—HpaI recognition site (GTTAAC)—multiple adenosines (poly A-50)—HpaI recognition site (GTTAAC)—multiple adenosines (MA-50)—HpaI recognition site (GTTAAC)—CVB3 5′ UTR (SEQ ID NO: 2) as a first IRES element—expression enhancer sequence (ATGGCAGCTCAA) (SEQ ID NO: 29)—EcoRI recognition site and Kozak sequence (GATTC GACC)—R/L as a first ORF (SEQ ID NO: 17)—SacI recognition site (GAGCTC)—EMCV 5′ UTR (SEQ ID NO: 4) as a second IRES element—EcoRV-SalI-PacI recognition sites and Kozak sequences (GATATC GTCGAC TTAATTAA GACC) (SEQ ID NO: 34)—F/L as a second ORF (SEQ ID NO: 18)—SacII recognition site (CCGCGG)—CVB3 3′ UTR (SEQ ID NO: 8)—poly A 50—NotI recognition site (GCGGCCGC)—3′. The nucleic acid molecule fabricated in this Example will be referred as “pMA-CVB3-R/L-EMCV-F/L”.

### Example 11: Fabrication of Nucleic Acid Molecule of RNA Platform

An artificial nucleic acid molecule of RNA platform including multiple viral IRES elements derived from CrPV and EMCV by repeating the same process as Example 9 except using firefly luciferease ORF (F/L) as the second ORF. The template DNA has the following sequence:

5′-BamHI recognition site (GGATCC)—T7 promoter (SEQ ID NO: 14)—PMeI recognition site (GTTTAAAC)—CrPV IGR IRES (SEQ ID NO: 6) as a first IRES element—Start codon (CCT GCT)—EcoRI recognition site (GAATTC)—R/L as a first ORF (SEQ ID NO: 17)—SacI recognition site (GAGCTC)—EMCV 5′ UTR (SEQ ID NO: 4) as a second IRES element—EcoRV-SalI-PacI recognition sites and Kozak sequences (GATATC GTCGAC TTAATTAA GACC) (SEQ ID NO: 34)—F/L as a second ORF (SEQ ID NO: 18)—SacII recognition site (CCGCGG)—SV40 late polyadenylation signal sequence (SEQ ID NO: 15)—NotI recognition site (GCGGCCGC)—3′. The nucleic acid molecule fabricated in this example will be referred as “pCrPV-R/L-EMCV-F/L”

### Experimental Example 3: Measurement of Expression Efficiency of Nucleic Acid Molecule

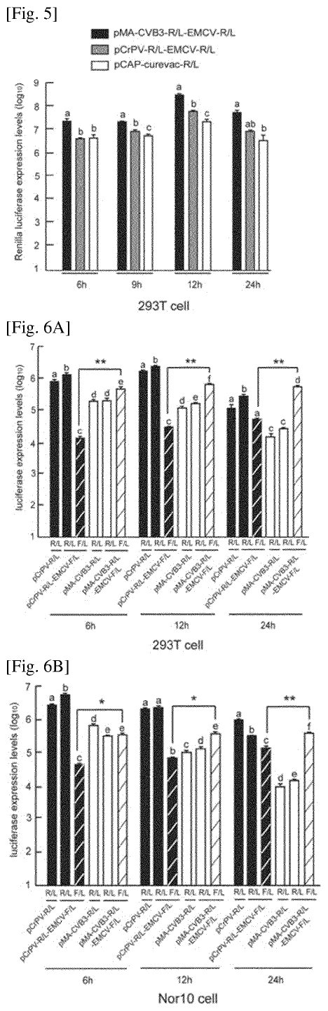

Each of nucleic acid molecules in Examples 8 to 9 (pMA-CVB3-R/L-EMCV-R/L, and pCrPV-R/L-EMCV-R/L) and Comparative Example 1 (pCAP-curevac-R/L) was transfected into 293T cells and mouse Nor10 muscle fibroblasts. Mouse Nor10 muscle fibroblasts were obtained from the Korean Cell Line Bank (Korea). 293T cells and Nor10 fibroblasts were maintained in Dulbecco's Modified Eagle's Medium (Hyclone, GE Healthcare, UK) containing 10% FBS and 1% amino acids. Expression levels of Renilla luciferase (R/L) in the cell line were measured as the same process as Experimental Example 1. FIG. 5 illustrates expression levels of R/L in 295T cells. As illustrated in FIG. 5, pMA-CVB3-R/L-EMCV-R/L showed an express level that was much higher than that of CrPV-R/L-EMCV-R/L, and even higher than that of pCAP-curevac-R/L in 293T cells. Besides, pCrPV-R/L-EMCV-RL showed an expression level that was higher than that of pCAP-curevac-R/L in 293T cells.

Then, each of nucleic acid molecules in Example 5 (pCrPV-R/L0, Example 7 (pMA-CVB3-R/L), Examples 10 to 11 (pCrPV-R/L-EMCV-F/L and pMA-CVB3-R/L-EMCV-F/L) was transfected into 293T cells mouse Nor10 muscle fibroblasts. Mouse Nor10 muscle fibroblasts were obtained from the Korean Cell Line Bank (Korea). 293T cells and Nor10 fibroblasts were maintained in Dulbecco's Modified Eagle's Medium (Hyclone, GE Healthcare, UK) containing 10% FBS and 1% amino acids. Expression levels of Renilla luciferase (R/L) in the cell line were measured as the same process as Experimental Example 1. FIGS. 6A and 6B illustrate expression levels of R/L and F/L in 295T cells and Nor10 cells. As illustrated in FIGS. 6A and 6B, the expression of R/L from pCrPV-R/L-EMCV-F/L and pMA-CVB3-R/L-EMCV-F/L was not significantly different from that observed from pCrPV-R/L and pMA-CVB3-R/L. However, the expression level of F/L regulated by the EMCV IRES was higher in pMA-CVB3-R/L-EMCV-F/L than it was in pCrPV-R/L-EMCV-F/L in Nor10 and 293T cells.

### Example 12: Fabrication of Nucleic Acid Molecule of RNA Platform

An artificial nucleic acid molecule of RNA platform including a viral IRES element derived from CVB3 was fabricated by repeating the same process as Example 7 except using a template DNA including only multi-cloning site (MCS) without ORF (R/L). The template DNA has the following ordered nucleotides:

5′-BamHI recognition site (GGATCC)—T7 promoter (SEQ ID NO: 14)—multiple adenosines (MA-50)—CVB3 5′ UTR (SEQ ID NO: 2) as IRES element—expression enhancer sequence (ATGGCAGCTCAA) (SEQ ID NO: 29)—MCS of EcoRI-ClaI-PacI-SacI recognition sites (GAATTC ATCGAT TTAATTAA GAGCTC)(SEQ ID NO: 35)—CVB3 3′ UTR (SEQ ID NO: 8)—poly A 50—NotI recognition site (GCGGCCGC)—3′. The nucleic acid molecule fabricated in this Example will be referred as “pMA-CVB3”.

### Example 13: Fabrication of Nucleic Acid Molecule of RNA Platform

An artificial nucleic acid molecule of RNA platform including a viral IRES element derived from EMCV was fabricated by repeating the same process as Example 3 except using a template DNA including multiple adenosines (MA 50) inserted between T7 promoter and EMCV 5′ UTR and only MCS without ORF (R/L). The template DNA has the following ordered nucleotides:

5′-EcoRI recognition site (GAATTC)—T7 promoter (SEQ ID NO: 14)—multiple adenosines (MA-50)—EMCV 5′ UTR (SEQ ID NO: 4) as an IRES element—MCS of BamHI-ClaI-PacI-SacI recognition sites (GGATCC ATCGAT TTAATTAA GAGCTC)(SEQ ID NO: 36)—EMCV 3′ UTR (SEQ ID NO: 9)—poly A 50—NotI recognition site (GCGGCCGC)—3′. The nucleic acid molecule fabricated in this Example will be referred as “pMA-EMCV”

### Example 14: Fabrication of Nucleic Acid Molecule of RNA Platform

An artificial nucleic acid molecule of RNA platform including a viral IRES element derived from CrPV was fabricated by repeating the same process as Example 5 except using a template DNA including multiple adenosines (MA-50) inserted between T7 promoter and CrPV IGR IRES and only MCS without ORF (R/L). The template DNA has the following ordered nucleotides:

5′-BamHI recognition site (GGATCC)—T7 promoter (SEQ ID NO: 14)—multiple adenosines (MA-50)—CrPV IGR IRES (SEQ ID NO: 6) as IRES element—MCS of BamHI-EcoRI-PacI-SacI recognition sites (GGATCC GAATTC TTAATTAA GAGCTC)(SEQ ID NO: 37)—SV40 late polyadenylation signal sequence (SEQ ID NO: 15)—NotI recognition site (GCGGCCGC)—3′. The nucleic acid molecule fabricated in this example will be referred as “pMA-CrPV”

### Example 15: Fabrication of Nucleic Acid Molecule of RNA Platform

An artificial nucleic acid molecule of RNA platform including a viral IRES element derived from CVB3 was fabricating by repeating the same process as Example 12 except using a template DNA including multiple thymidines (MT-50) instead between T7 promoter and EMCV 5′ UTR. The template DNA has the following ordered nucleotides:

5′-BamHI recognition site (GGATCC)—T7 promoter (SEQ ID NO: 14)—multiple thymidines (MT-50)—CVB3 5′ UTR (SEQ ID NO: 2) as IRES element—expression enhancer sequence (ATGGCAGCTCAA) (SEQ ID NO: 29)—MCS of EcoRI-Cle-PacI-SacI recognition sites (GAATTC ATCGAT TTAATTAA GAGCTC)(SEQ ID NO: 35)—CVB3 3′ UTR (SEQ ID NO: 8)—poly A 50—NotI recognition site (GCGGCCGC)—3′. The nucleic acid molecule fabricated in this Example will be referred as “pMT-CVB3”.

### Example 16: Fabrication of Nucleic Acid Molecule of RNA Platform

An artificial nucleic acid molecule of RNA platform including a viral IRES element derived from EMCV was fabricating by repeating the same process as Example 13 except using a template DNA including multiple thymidines (MT-50) instead between T7 promoter and EMCV 5′ UTR. The template DNA has the following ordered nucleotides:

5′-EcoRI recognition site (GAATTC)—T7 promoter (SEQ ID NO: 14)—multiple thymidines (MT-50)—EMCV 5′ UTR (SEQ ID NO: 4) as an IRES element—MCS of BamHI-ClaI-PacI-SacI recognition sites (GGATCC ATCGAT TTAATTAA GAGCTC)(SEQ ID NO: 36)—EMCV 3′ UTR (SEQ ID NO: 9)—poly A 50—NotI recognition site (GCGGCCGC)—3′. The nucleic acid molecule fabricated in this Example will be referred as “pMT-EMCV”

### Example 17: Fabrication of Nucleic Acid Molecule of RNA Platform

An artificial nucleic acid molecule of RNA platform including a viral IRES element derived from CrPV was fabricating by repeating the same process as Example 14 except using a template DNA including multiple thymidines (MT-50) instead between T7 promoter and CrPV IGR IRES. The template DNA has the following ordered nucleotides:

5′-BamHI recognition site (GGATCC)—T7 promoter (SEQ ID NO: 14)—multiple thymidines (MT-50)—CrPV IGR IRES (SEQ ID NO: 6) as IRES element—MCS of BamHI-EcoRI-PacI-SacI recognition sites (GGATCC GAATTC TTAATTAA GAGCTC)(SEQ ID NO: 37)—SV40 late polyadenylation signal sequence (SEQ ID NO: 15)—NotI recognition site (GCGGCCGC)—3′. The nucleic acid molecule fabricated in this example will be referred as “pMT-CrPV”

### Example 18: Fabrication of Nucleic Acid Molecule of RNA Platform

An artificial nucleic acid molecule of RNA platform including a viral IRES element derived from CVB3 was fabricated by repeating the same process as Example 6 except using a template DNA including only multi-cloning site (MCS) without ORF (R/L). The template DNA has the following ordered nucleotides:

5′-BamHI recognition site (GGATCC)—T7 promoter (SEQ ID NO: 14)—CVB3 5′ UTR (SEQ ID NO: 2) as IRES element—expression enhancer sequence—MCS of SalI-EcoRV-SacII-PvuI recognition sites (GTCGAC GATATC CCGCGG CGATCG)(SEQ ID NO: 38)—CVB3 3′ UTR (SEQ ID NO: 8)—poly A 50—NotI recognition site (GCGGCCGC)—3′. The nucleic acid molecule fabricated in this Example will be referred as “pCVB3”.

### Example 19: Fabrication of Nucleic Acid Molecule of RNA Platform

An artificial nucleic acid molecule of RNA platform including a viral IRES element derived from EMCV was fabricated by repeating the same process as Example 3 except using a template DNA including only multi-cloning site (MCS) without ORF (R/L). The template DNA has the following ordered nucleotides:

5′-EcoRI recognition site (GAATTC)—T7 promoter (SEQ ID NO: 14)—EMCV 5′ UTR (SEQ ID NO: 4) as an IRES element—MCS of BamHI-SacI-SalI-PvuI recognition sites (GGATCC CCGCGG GTCGAC CGATCG)(SEQ ID NO: 39)—EMCV 3′ UTR (SEQ ID NO: 9)—poly A 50—NotI recognition site (GCGGCCGC)—3′. The nucleic acid molecule fabricated in this Example will be referred as “p-EMCV”

### Example 20: Fabrication of Nucleic Acid Molecule of RNA Platform

An artificial nucleic acid molecule of RNA platform including a viral IRES element derived from CrPV was fabricated by repeating the same process as Example 5 except using a template DNA including only multi-cloning site (MCS) without ORF (R/L). The template DNA has the following ordered nucleotides:

5′-BamHI recognition site (GGATCC)—T7 promoter (SEQ ID NO: 14)—multiple thymidines (MT-50)—CrPV IGR IRES (SEQ ID NO: 6) as IRES element—MCS of BamHI-EcoRI-PacI-SacI recognition sites (GGATCC GAATTC TTAATTAA GAGCTC)(SEQ ID NO: 37)—SV40 late polyadenylation signal sequence (SEQ ID NO: 15)—NotI recognition site (GCGGCCGC)—3′. The nucleic acid molecule fabricated in this example will be referred as “pCrPV”

### Experimental Example 4: Immunoassay of Nucleic Acid Molecule

Immunoassay using the nucleic acid molecules synthesized in Examples 12 to 20 was performed to investigate the molecule of RNA platform as an adjuvant. C57BL/6 mice aged 6 weeks were inoculated by intramuscular injection, 2 times at 1 week interval, with 20 MERS spike (S) soluble protein vaccine (SEQ ID NO: 21; SK bioscience, South Korea) and alum 120 μg with or without 5 μg of each of the nucleic acid molecule as indicated in Table 1 below.

TABLE 1
Formulation of RNA and MERS protein

| Group | Substrate | Dose(per mouse) | |
|---|---|---|---|
| G1 | PBS | 60 μL | |
| G2 | MERS protein | 5 μg | SK MERS |
| G3 | MERS protein + Alum | Protein 5 μg; Alum 120 μg | SK MERS |
| G4 | MERS protein + Alum + RNA | Protein 5 μg; RNA 20 μg | pMA-CVB3 |
| G5 | MERS protein + Alum + RNA | Protein 5 μg; RNA 20 μg | pMA-EMCV |
| G6 | MERS protein + Alum + RNA | Protein 5 μg; RNA 20 μg | pMA-CrPV |
| G7 | MERS protein + Alum + RNA | Protein 5 μg; RNA 20 μg | PMT-CVB3 |
| G8 | MERS protein + Alum + RNA | Protein 5 μg; RNA 20 μg | pMT-EMCV |
| G9 | MERS protein + Alum + RNA | Protein 5 μg; RNA 20 μg | pMT-CrPV |
| G10 | MERS protein + Alum + RNA | Protein 5 μg; RNA 20 μg | pCVB3 |
| G11 | MERS protein + Alum + RNA | Protein 5 μg; RNA 20 μg | pEMCV |
| G12 | MERS protein + Alum + RNA | Protein 5 μg; RNA 20 μg | pCrPV |

Blood samples from all experimental mice were taken at two weeks after second immunization, and measured IgG1 and IgG2c within the collected mouse serum using ELISA. Antigen-specific IgG1 and IgG2c in mouse serum were measured by ELISA. The 96-well plates (Corning®) were coated with 50 ng/well MERS spike soluble protein vaccine overnight at 4° C. After incubation, the wells were blocked with 200 μL blocking buffer (PBS-1% BSA) for 1 hour at room temperature. Diluted serum samples were added to the plates and incubated for 1 hour at room temperature. After incubation, the wells were washed three times with 200 μL PBS-T (PBS-0.05% tween 20). The anti-mouse IgG1 and IgG2c-HRP (Bethyl, Invitrogen, and Novus, respectively) diluted 1/1000-1/10000 in PBS were added to the plate and incubated for 1 hour at room temperature. After three washes with PBS-T, TMB substrate was added and incubated for 15 min and then 2N H2SO4 was used to stop the reaction. The O.D. values were measured at 450 nm using a GloMax explorer 817 microplate reader (Promega).

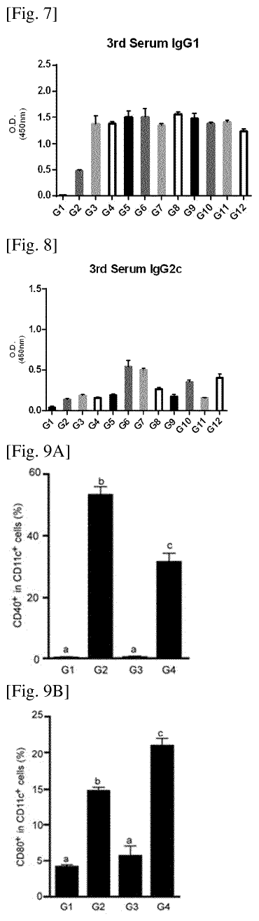

As illustrated in FIG. 7, IgG1 levels, which indicates a predominantly Th2 immune response, were higher in alum-formulated group (G3) and alum and RNA formulated groups (G4-G12) compared to only MERS S soluble protein group (G2). This means that nucleic acid molecules including only viral IRES elements without any coding region may act as an immune stimulator that induce Th2 immune response, i.e. humoral immune response. In contrast, as illustrated in FIG. 8, IgG2c levels, which indicates a predominantly Th1 immune response, were higher in alum and RNS formulated groups, particularly G6 (alum+pMA-CrPV), G7 (alum+pMT-CVB3), G10 (alum+pCVB3) and G12 (alum+pCrPV) compared to only alum formulated group (G3). Considering the results in FIGS. 7 and 8, the nucleic acid molecules including only viral IRES elements without any coding region may act as an immune stimulator that induce T cell activation via Th1 immune response and Th2 immune response and showed excellent adjuvanicity.

### Experimental Example 5: Immunoassay of Nucleic Acid Molecule

Immunoassay using the nucleic acid molecule in Example 5 (pCrPV-R/L) was performed to investigate the molecule as the immune-stimulatory component such as adjuvant. Female C57BL/6 mice were purchased from Dae-Han Bio-Link (Korea). Bone marrow cells from C57BL/6 mice were placed into 100 mm cell culture dishes at 3×107 cells/mL in dendritic cell (DC) conditioned medium, which consisted of RPMI (Hyclone Laboratories) supplemented with 10% heat-inactivated FBS (Life Technologies), 2.05 mL L-glutamine (Hyclone), and anti-anti solution (Gibco) containing 10 ng/mL recombinant murine GM-CSF (BD PharMingen), 1 ng/mL recombinant murine IL-4 (BD PharMingen) and 0.05 mM β-mercaptoethanol. Half of DC medium was replaced on day 3 and 6, and the cells were harvested on day 7. The mBMDCs were differentiated completely on day seven, and 5×106 cells/mL mBMDCs were dispensed again to well-plate. The RNA, i.e. pCrPV-R/L was formulated with protamine (Sigma Aldrich) with a ratio of 2:1 and then completely-differentiated mBMDCs were treated with pCrPV-R/L formulated with protamine to analyze cytokines secreted thereby using immunoassay. PBS (phosphorated buffered saline) treated mBMDCs, LPS (lipopolysaccharide) treated mBMDCs, and only protamine treated mBMDCs were uses as controls as indicated in Table 2 below.

TABLE 2
Formulation of RNA

| Group | G1 | G2 | G3 | G4 |
|---|---|---|---|---|
| LPS | − | + | − | − |
| pCrPV-R/L | − | − | − | + |
| Protamine | − | − | + | + |

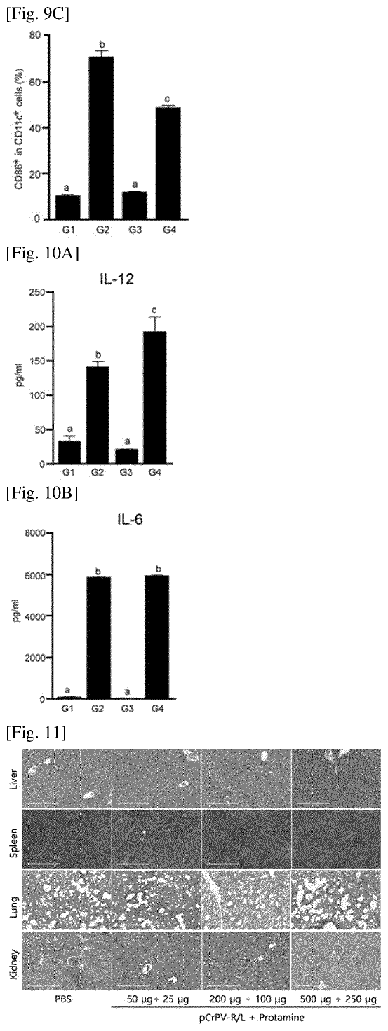

Flow cytometry assay was performed as follows: for surface staining, mBMDCs were stained with the following antibodies for 15 minutes at room temperature, CD40-APC (clone 1C10), CD80-PE (clone 16-10A1) and CD86 (clone GL1). The stained cells were analyzed using a FACS Accuri Flow Cytometer (BD Bioscience). As illustrated in FIGS. 9A to 9C, G4 (pCrPV-R/L formulated with protamine) activates dendritic cells such as CD11c+CD40+ cells, CD11c+CD80+ and CD11c+CD86+ cells.

ELISA assay was performed to measure levels of cytokines in the culture supernatant using ELISA kits (eBiosceince for IL-6 and IL-12 and Invitrogen for TNF-α) were used following the manufacturer's protocol. As illustrated in FIGS. 10A and 10B, G4 (pCrPV-R/L formulated with protamine) activates secretions of IL-12 and IL-6 each of which is a cytokine associated with Th1 immune response.

Besides, image processing was performed to investigate the tissue changes by inoculation of the nucleic acid molecules formulated with protamine. A laser-scanning intravital confocal microscope (IVM-C, IVIM Technology) was used to visualize kidney tissues, lung tissues, spleen tissues and liver tissues in mice. As illustrated in FIG. 11, there were any inflammations within the mice tissues treated with the nucleic acid molecules. This means that the nucleic acid molecule is degraded in the host rapidly and has high stabilities while it activates immune cells like LPS which causes strong inflammatory responses in the host in spite of the strong adjuvanicity.

### Example 21: Fabrication of Nucleic Acid Molecule of RNA Platform

An artificial nucleic acid molecule of RNA platform including a viral IRES element derived from CrPV was fabricated by repeating the same process as Example 5 except using a template DNA including nucleotides (SEQ ID NO: 19) encoding MERS S soluble protein in place of Renilla luciferase as an ORF. The template DNA has the following ordered nucleotides:

5′-KpnI recognition site (GGTATC)—T7 promoter (SEQ ID NO: 14)—CrPV IGR IRES (SEQ ID NO: 6) as IRES element—start codons (CCT GCT)—MERS S as ORF (SEQ ID NO: 20)—SV40 late polyadenylation signal sequence (SEQ ID NO: 15)—NotI recognition site (GCGGCCGC)—3′. The nucleic acid molecule fabricated in this example will be referred as “pCrPV-MERS”

### Experimental Example 6: Immunoassay of Nucleic Acid Molecule

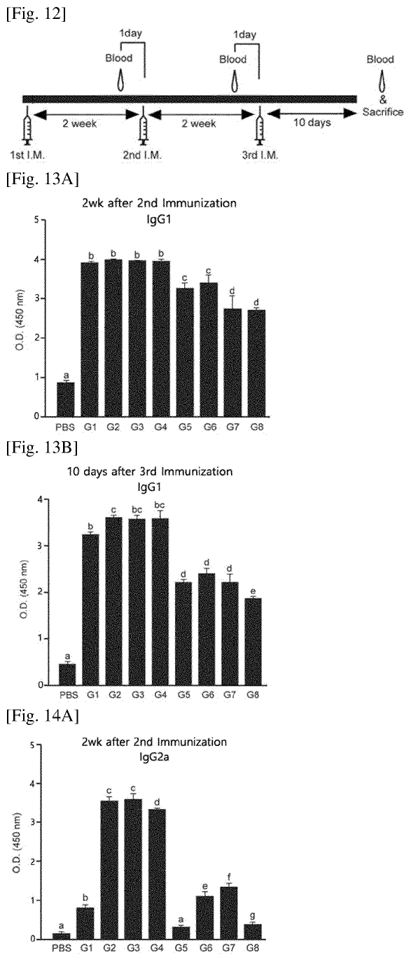

Immunoassay using the nucleic acid molecules synthesized in Example 21 was performed to investigate the molecule of RNA platform as an adjuvant as Experimental Example 4. C57BL/6 mice aged 6 weeks were inoculated by intramuscular injection or intranasal injection, three times at 2 weeks interval with following formulations; 5 μg/mice of MERS spite (S) soluble protein vaccine formulated with or without adjuvant, 20 μg/mice of RNA (pCrPV-MERS) pre-formulated with 10 μg of protamine and/or 120 μg/mice of alum (Thermo Scientific) as indicated in Table 3 below and FIG. 12. 100 μL/mice of immunogen was injected in intramuscular injection and 45 μL/mice of immunogen was injected in intranasal injection.

TABLE 3
Formulation of RNA and MERS Protein

| | | Intramuscular (I.M.) | | | | Intranasal (I.N.) | | | |
|---|---|---|---|---|---|---|---|---|---|
| | PBS | G1 | G2 | G3 | G4 | G5 | G6 | G7 | G8 |
| MERS S protein | − | + | + | + | + | + | + | + | + |
| Alum | − | + | + | − | − | + | + | − | − |
| pCrPV-MERS | − | − | + | + | − | − | + | + | − |
| pCrPV-R/L | − | − | − | − | + | − | − | − | + |

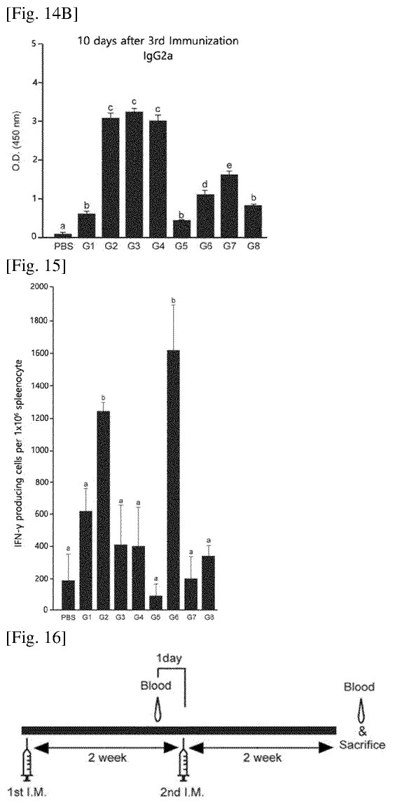

We performed immunoassays using sera of mice immunized with MERS S soluble protein vaccines with or without RNA. FIGS. 13A, 13B, 14A and 14C illustrate MERS 5-specific IgG levels by ELISA. As illustrated in FIG. 13A, IgG1 levels, which indicates a predominantly Th2 immune response, were slightly higher in groups immunized intramuscularly (G1-G4) than groups immunized intranasally (G5-G8) after second immunization. IgG1 is not induced in PBS treated group. Besides, as illustrated in FIG. 13B, IgG1 levels were higher about 1.5 to two times in groups immunized intramuscularly (G1-G4) as groups immunized intranasally (G5-G8). IgG1 levels in the third sera were high in all groups immunized with PCrPV-MERS (G2, G3, G6 and G7).

On contrary, as illustrated in FIG. 14A, IgG2a levels, which indicates predominantly Th1 immune response, were extremely higher in groups immunized intramuscularly (G2-G4) than groups immunized intranasally in the 2nd sera of the mice. Also, IgG2a levels were very low in groups immunized only with MERS S soluble protein vaccine (G1 and G7). This means that the protein vaccine formulated with only alum does not induce Th1 immune response, i.e. cell-mediated immune response.

Also, as illustrated in FIG. 14B, IgG2a levels in the 3rd sera of the mice was generally similar IgG2a level in the 2nd sera. However, IgG2a levels in the 3rd sera of the mice was extremely higher in groups immunized intramuscularly (G2-G4) than groups immunized intranasally (G6-G8), unlike IgG1 level in the 3rd serum of the mice. Besides, Groups immunized only with MERS S soluble protein vaccine showed very low IgG2a levels (G1 and G5), which re-confirmed that the protein vaccine formulated with only alum does not induce Th1 immune response, i.e. cell-mediated immune response. Such results indicate that protein vaccines or antigens induce Th2 immune response with regard to humoral immune responses to produce antibodies while they induce Th1 immune response poorly with regard to cell-mediated immune responses. However, Immunization using pharmaceutically active ingredients such as protein or peptide vaccines or antigens formulated with the nucleic acid molecule of the present disclosure as the adjuvant can induce Th1 immune response as well as Th2 immune response. This means that immunization using the protein and peptides as immunogens together with the nucleic acid molecules is an excellent strategy for inducing balanced immune responses caused by the immunogens.

Spleenocytes (1×106 cells/100 uL/well) were transferred to a 96-well plate, and stimulated with 2 ug/well of MERS Spike T-cell epitope for 48 hours at 37° C. ELISPOT detection of IFN-γ was performed according to the manufacture's instruction (Mabtech). As illustrated in FIG. 15, Groups 2 and 6, which immunized with MERS S protein vaccine together with alum and RNA (pCrPV-MERS), showed the highest MERS Spike T cell epitope-specific IFN-γ secreted T cell populations. Considering such results, in case of immunizing peptides or proteins an immunogens formulated with the nucleic acid molecule encoding the peptides or proteins, the encoded ORF induce cytotoxic T-cell response caused by the immunogens.

### Example 22: Fabrication of Nucleic Acid Molecule of RNA Platform

An artificial nucleic acid molecule of RNA platform including multiple viral IRES elements derived from CrPV and EMCV by repeating the same process as Example 9 except using a template DAN including nucleotides (SEQ ID NO: 19) encoding MERS spike soluble protein in place of Renilla luciferase as an ORF. The template DNA has the following ordered nucleotides:

5′-BamHI recognition site (GGATCC)—T7 promoter (SEQ ID NO: 14)—PMeI recognition site (GTTTAAAC)—CrPV IGR IRES (SEQ ID NO: 6) as a first IRES element—Start codon (CCT GCT)—EcoRI recognition site (GAATTC)—MERS S protein as a first ORF (SEQ ID NO: 19)—SacI recognition site (GAGCTC)—EMCV 5′ UTR (SEQ ID NO: 4) as a second IRES element—EcoRV-SalI-PacI recognition sites and Kozak sequences (GATATC GTCGAC TTAATTAA GACC) (SEQ ID NO: 34)—MERS S protein as a second ORF (SEQ ID NO: 19)—SacII recognition site (CCGCGG)—SV40 late polyadenylation signal sequence (SEQ ID NO: 15)—NotI recognition site (GCGGCCGC)—3′. The nucleic acid molecule fabricated in this example will be referred as “pCrPV-MERS-EMCV-MERS”

### Experimental Example 7: Immunoassay of Nucleic Acid Molecule

Immunoassay using the nucleic acid molecules synthesized in Example 22 was performed to investigate the molecule of RNA platform as an adjuvant as Experimental Example 6. C57BL/6 mice aged 6 weeks were inoculated by intramuscular injection into the upper thigh, two times at two weeks interval with the following formulations; 5 μg/mice of MERS spite (S) soluble protein vaccine formulated with or without adjuvant, 20 μg/mice of RNA (pCrPV-MERS-EMCV-MERS) pre-formulated with 10 μg of protamine and/or 120 μg/mice of alum (Thermo Scientific) as indicated in Table 4 below and FIG. 16.

TABLE 4
Formulation of RNA and MERS Protein

IM

| Group | 1^{st} | 2^{nd} |
|---|---|---|
| PBS | PBs | PBS |
| S1-2 | MERS S/Alum | MERS S/Alum |
| S1-4 | RNA + MERS S/Alum | RNA + MERS S/Alum |

We performed immunoassays using sera of mice immunized with MERS S soluble protein vaccines with or without RNA (pCrPV-MERS-EMCV-MERS). MERS-CoV specific neutralizing antibody levels after 1st and 2nd immunization were determined by PRNT after 2 weeks after each immunization. Serum of MCRS-CoV infected mice were serially diluted from 1:10 to 1:5120 with serum-free media. The virus-serum mixture was prepared by mixing 100 PFU MERS-CoV with the diluted serum samples and incubated at 37° C. for 1 hour. The virus-antibody mixture was inoculated onto Vero cells. The plates were incubated for 1 h at 37° C. in 5% CO2. After virus adsorption, agar overlay media was added and the plates were incubated at 37° C. in 5% CO2 for 3 days. The cells were stained with 0.1% crystal violet solution (Sigma). Plaques were counted with the naked eye. The percentage neutralization represented the reduction value, which was calculated as 100× the number of plaques in the 100 PFU virus-infected well/the number of plaques in the virus-serum mixture infected well. As illustrated in FIG. 17, groups S1-2 (immunized with MERS S protein+alum) and S1-4 (immunized RNA as well as MERS S protein and alum) showed high MERS-CoV spike protein-specific neutralizing antibody values after 1st and 2nd immunizations.

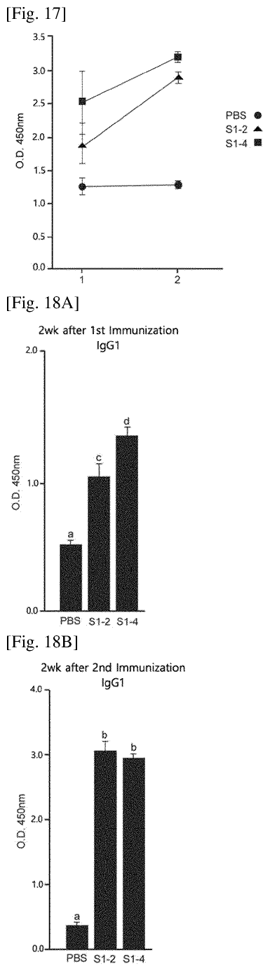

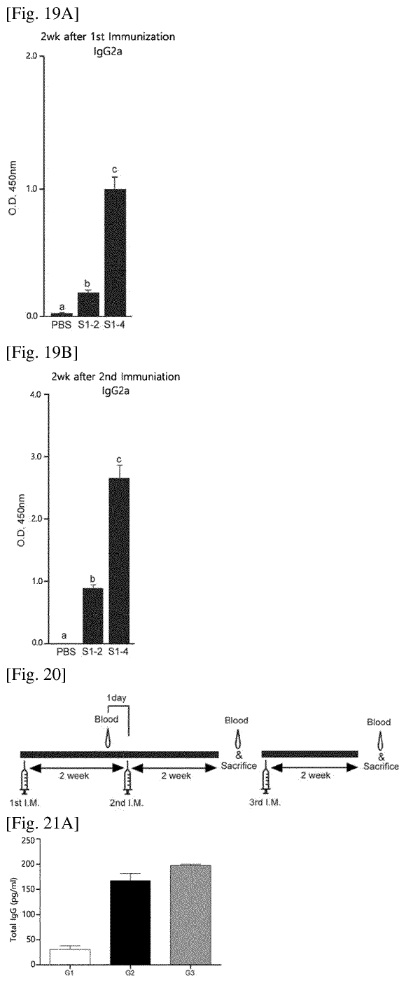

Also, as indicated in FIGS. 18A and 18B, IgG1 levels, which indicates a predominantly Th2 immune response, were high in both groups S1-2 and 51-4. Particularly, as indicated in FIGS. 19A and 19B, IgG2a levels, which indicates predominantly Th1 immune response, were extremely higher in a group immunized with RNA and MERS S protein formulated with alum (S1-4) than a group immunized with only MERS S protein formulated with alum (S1-2). Such result indicates the RNA (pCrPV-MERS-EMCV-MERS) induces excellent Th1 immune response.

### Example 23: Fabrication of Nucleic Acid Molecule of RNA Platform

An artificial nucleic acid molecule of RNA platform including a viral IRES element derived from CrPV was fabricated by repeating the same process as Example 21 except using a template DNA including nucleotides (SEQ ID NO: 21) encoding L1 of HPV-16 in place of MERS S soluble protein as an ORF. The template DNA has the following ordered nucleotides:

5′-KpnI recognition site (GGTATC)—T7 promoter (SEQ ID NO: 14)—CrPV IGR IRES (SEQ ID NO: 6) as IRES element—start codons (CCT GCT)—L1 of HPV-16 as ORF (SEQ ID NO: 21)—SV40 late polyadenylation signal sequence (SEQ ID NO: 15)—NotI recognition site (GCGGCCGC)—3′. The nucleic acid molecule fabricated in this example will be referred as “pCrPV-HPV16”

### Example 24: Fabrication of Nucleic Acid Molecule of RNA Platform

An artificial nucleic acid molecule of RNA platform including a viral IRES element derived from CrPV was fabricated by repeating the same process as Example 21 except using a template DNA including nucleotides (SEQ ID NO: 22) encoding L1 of HPV-18 in place of MERS S soluble protein as an ORF. The template DNA has the following ordered nucleotides:

5′-KpnI recognition site (GGTATC)—T7 promoter (SEQ ID NO: 14)—CrPV IGR IRES (SEQ ID NO: 6) as IRES element—start codons (CCT GCT)—L1 of HPV-18 as ORF (SEQ ID NO: 22)—SV40 late polyadenylation signal sequence (SEQ ID NO: 15)—NotI recognition site (GCGGCCGC)—3′. The nucleic acid molecule fabricated in this example will be referred as “pCrPV-HPV18”

### Experimental Example 8: Immunoassay of Nucleic Acid Molecule

Immunoassay using the nucleic acid molecules synthesized in Examples 23 and 24 to investigate the molecule of RNA platform as an adjuvant as Experimental Example 4. BALB/c mice (Dae-Han Bio-Link) mice aged 6 weeks were inoculated by intramuscular injection into the upper thigh, three times at two weeks interval with the following formulations; 6 μg/mice of 10 value HPV vaccines (mixed with 6, 11, 16, 18, 31, 33, 35, 45, 52 and 58 L1; SK Bioscience) as the virus like particle (VLPs) vaccine with or without adjuvant, pre-formulated with 10 μg of protamine and/or 120 μg/mice of alum (Thermo Scientific) as indicated in Table 5 below and FIG. 20. 100 μL/mice of immunogen was injected intramuscularly.

TABLE 5
Formulation of RNA and HPV VLPs

| Group | G1 | G2 | G3 |
|---|---|---|---|
| HPV VLPs + Alum | − | + | + |
| pCrPV-HPV16, pCrPV-HPV18 | − | − | + |
| Protamine | − | − | + |

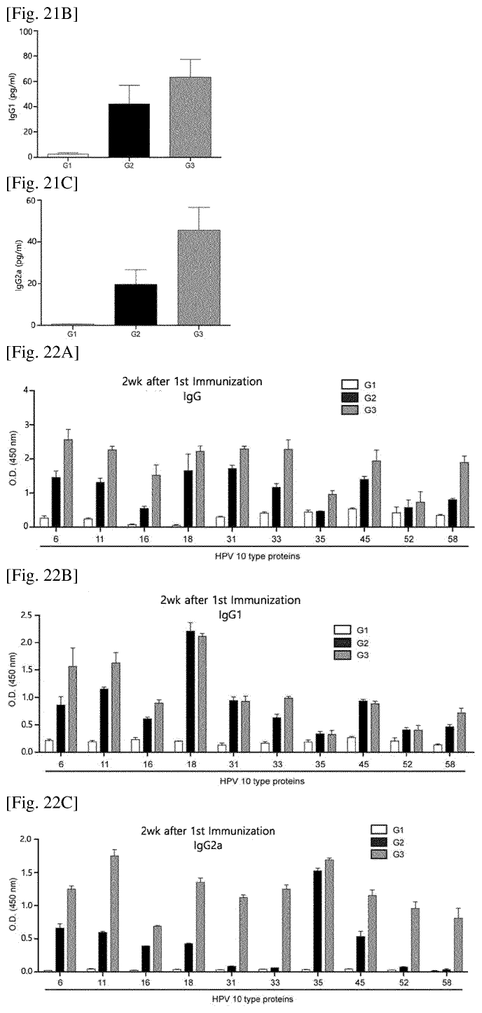

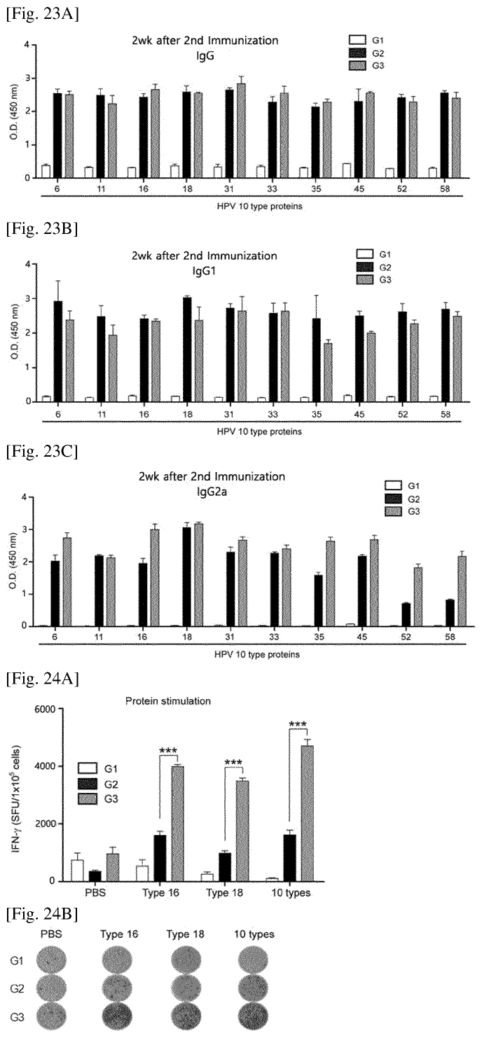

We performed immunoassays using sera of mice immunized with HPV VLPs with or without RNA (pCrPV-HPV16 and pCrPV-HPV18). As illustrated in FIGS. 21A to 23C, total IgG level, which indicates Th1 immune response as an innate immune response), IgG1 levels and IgG2a levels were generally higher in a group immunized with HPV VLPs formulated with RNA and protamine (G3) than a group immunized with HPV VLPs only (G2). Such results indicate that the RNA (pCrPV-HPV16 and/or pCrPV-HPV18) induces excellent Th1 immune response.

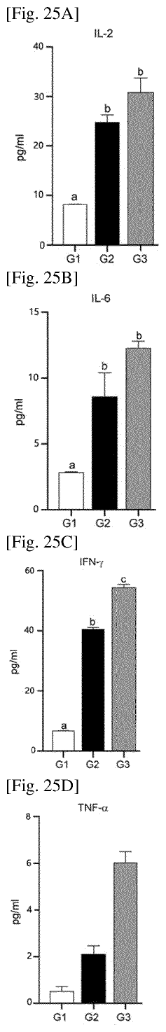

Spleenocytes (1×106 cells/100 uL/well) were transferred to a 96-well plate, and stimulated with 2 ug/well of MERS Spike T-cell epitope for 48 hours at 37° C. ELISPOT detection of IFN-γ was performed according to the manufacture's instruction (Mabtech) and ELISA assay for IL-2, IL-7, IFN-γ and TNF-α, each of which is a cytokine associated with Th1 immune response, was performed. As illustrated in FIGS. 24A, 24B, the group immunized with HPV VLPs formulated with RNA and protamine (G3) showed much higher HPV L1 protein-specific IFN-γ secreted T cell population. This result indicates that the RNA induce CTL effectively. Also, as indicated in FIGS. 25A to 25D, the group immunized with HPV VLPs formulated with RNA and protamine (G3) much activated secretion of Th1 immune response associated cytokines, i.e. IFN-γ, IL-2, IL-6 and TNF-α than the group immunized only with HPV VLPs (G2).

### Example 25: Fabrication of Nucleic Acid Molecule of RNA Platform

An artificial nucleic acid molecule of RNA platform including multiple viral IRES elements derived from CrPV and EMCV by repeating the same process as Example 9 except using a template DAN including nucleotides (SEQ ID NO: 25) encoding haemagglutin (HA) of influenza virus in place of Renilla luciferase as ORF. The template DNA has the following nucleotides:

5′-BamHI recognition site (GGATCC)—T7 promoter (SEQ ID NO: 14)—PMeI recognition site (GTTTAAAC)—CrPV IGR IRES (SEQ ID NO: 6) as a first IRES element—Start codon (CCT GCT)—EcoRI recognition site (GAATTC)—HA as a first ORF (SEQ ID NO: 23)—SacI recognition site (GAGCTC)—EMCV 5′ UTR (SEQ ID NO: 4) as a second IRES element—EcoRV-SalI-PacI recognition sites and Kozak sequences (GATATC GTCGAC TTAATTAA GACC) (SEQ ID NO: 34)—HA as a second ORF (SEQ ID NO: 23)—SacII recognition site (CCGCGG)—SV40 late polyadenylation signal sequence (SEQ ID NO: 15)—NotI recognition site (GCGGCCGC)—3′. The nucleic acid molecule fabricated in this example will be referred as “pCrPV-HA-EMCV-HA”

### Example 26: Fabrication of Nucleic Acid Molecule of RNA Platform

An artificial nucleic acid molecule of RNA platform including multiple viral IRES elements derived from CVB3 and EMCV by repeating the same process as Example 8 except using a template DAN including nucleotides (SEQ ID NO: 23) encoding haemagglutin (HA) of influenza virus in place of Renilla luciferase as ORF. The template DNA has the following nucleotides:

5′-BamHI recognition site (GGATCC)—T7 promoter (SEQ ID NO: 14)—HpaI recognition site (GTTAAC)—multiple adenosines (poly A-50)—HpaI recognition site (GTTAAC)—multiple adenosines (MA-50)—HpaI recognition site (GTTAAC)—CVB3 5′ UTR (SEQ ID NO: 2) as a first IRES element—expression enhancer sequence (ATGGCAGCTCAA) (SEQ ID NO: 29)—EcoRI recognition site and Kozak sequence (GATTC GACC)—HA as a first ORF (SEQ ID NO: 23)—SacI recognition site (GAGCTC)—EMCV 5′ UTR (SEQ ID NO: 4) as a second IRES element—EcoRV-SalI-PacI recognition sites and Kozak sequences (GATATC GTCGAC TTAATTAA GACC) (SEQ ID NO: 34)—HA as a second ORF (SEQ ID NO: 23)—SacII recognition site (CCGCGG)—CVB3 3′ UTR (SEQ ID NO: 8)—poly A 50—NotI recognition site (GCGGCCGC)—3′. The nucleic acid molecule fabricated in this Example will be referred as “pMA-CVB3-HA-EMCV-HA”.

### Experimental Example 9: Immunoassay of Nucleic Acid Molecule

Immunoassay using the nucleic acid molecules synthesized in Examples 25 and 26 was performed to investigate the molecule of RNA platform as an adjuvant as Experimental Example 6. BALB/mice (Samtako Biokorea) aged 5 weeks were inoculated by intramuscular injection (I.M.) or electroporation injection (EP), three times at two week interval with the following formulations; 2.5×105 pfu/50 μL/mice of inactivated influenza virus vaccine (SEQ ID NO: 24; SKY Cellflu; iPR8) with or without adjuvant, 120 μg/mice of alum (Thermo Scientific), 50 μg/mice of RNA (pCrPV-HA-EMCV-HA or pCVB3-HA-EMCV-HA) pre-formulated with 250 μg of protamine and/or LNP as indicated in Table 6 below and FIG. 26. 100 μL/mice of immunogen was injected in intramuscular injection except using LNP (lipid nano particle) (120 μL/mice of immunogen), 45 μL/mice of immunogen was injected in electroporation injection.

TABLE 6
Formulation of RNA and Influenza Protein

| | | | | pCrPV-HA- | pMA-CVB3-HA- | | |
|---|---|---|---|---|---|---|---|
| Group | | iPR8 | Alum | EMCV-Ha | EMCV-HA | protamine | LNP |
| I.M. | PBS | − | − | − | − | − | − |
| | HA1 | + | − | − | − | − | − |
| | HA2 | + | + | − | − | − | − |
| | HA3 | + | − | + | − | − | − |
| | HA4 | + | − | − | + | − | − |
| EP | HA5 | − | − | + | − | − | − |
| | HA6 | − | − | + | − | + | − |
| | HA7 | − | − | − | + | − | − |
| | HA8 | − | − | − | + | + | − |
| I.M. | HA9 | − | − | + | − | − | + |
| | HA10 | − | − | − | + | − | + |

We performed immunoassays using sera of mice immunized with MERS S soluble protein vaccines with or without RNA (pCrPV-HA-EMCV-HA or pMA-CVB3-HA-EMCV-HA). Influenza specific neutralizing antibody levels after 1st immunization were determined by PRNT as Experimental Example 7. As illustrated in FIG. 27, Influenza virus-specific neutralizing antibodies were not induced in all groups except group “HA2”, which was immunized with iPR8 virus formulated with alum, after 1st immunization. However, groups HA′ (iPR8 virus), HA3 (iPR8+pCrPV-HA-EMCV-HA) and HA4 (iPR8+pMA-CVB3-HA-EMCV-HA) as well as HA2 showed increased influenza virus specific neutralizing antibodies by second immunization (1st boosting) and third immunization. These result meant that immunizing the nucleic acid molecules formulated with influenza vaccine and alum by intramuscular injection can induce enough antibodies by second immunization (1st boosting).

After eight days from the third immunization, influenza virus vaccine was challenged into the immunized mice. After four days from the challenge, Spleenocytes (1×106 cells/100 uL/well) were transferred to a 96-well plate, and stimulated with MHC I peptides (pR8 T cell epitope) and 2 ug/well of recombinant pR8 proteins and ELISPOT detection of the obtained HA specific Th1 cells was performed. As illustrated in FIG. 28, Group “HA2” (iPR8+alum), “HA3” and “HA4” (iPR8 virus+RNA) showed relatively high IFN-γ secreted T cell population in case of stimulating with MHC I peptide. Besides, groups “HA9” and “HA10”, each of which formulated with RNA and LNP, was somewhat high IFN-γ secreted T cell population compared to groups “HA5” and “HA6”, formulated with only RNA, or groups “HA7” and “HA8” formulated with RNA and protamine.

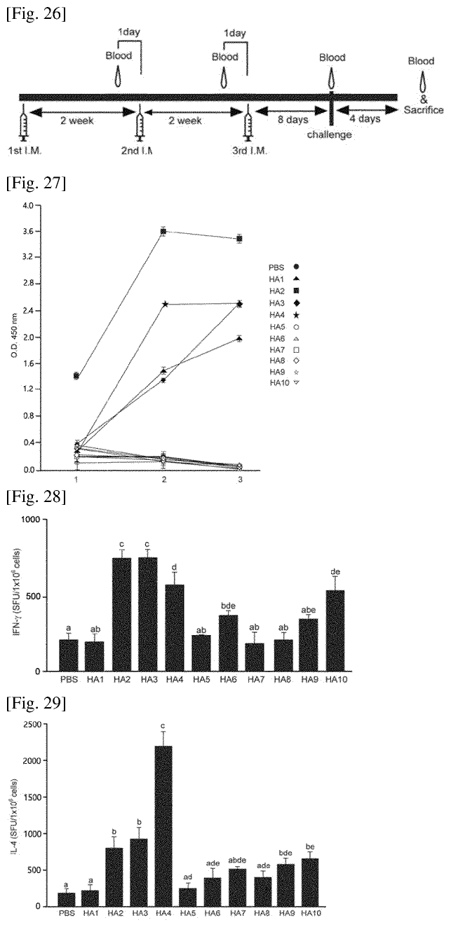

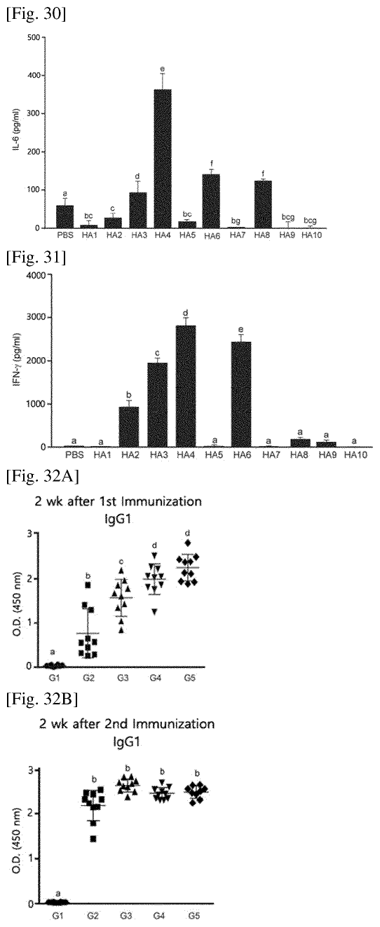

On the contrary, groups “HA3” and “HA4”, formulated with iPR8 virus and RNA, showed higher IL-4 secreted T cell population than group “HA2”, formulated with iPR8 virus and alum, as illustrated in FIG. 29. Besides, groups “HA3” and “HA4”, formulated with iPR8 virus and RNA, showed much higher IL-6 secreted T cell population that groups “HA5” and “HA6”, formulated with only RNA, and groups “HA7” and “HA8”, formulated with RNA and protamine, as illustrated in FIG. 30. Particularly, group “HA2”, formulated iPR8 virus and alum, showed relatively low IL-6 secreted T cell population in case of stimulating with peptides.

Also, as illustrated in FIG. 31, which shows ELISA assay, Group “HA3” and “HA4” formulated with iPR8 virus and RNA” showed extremely high IFN-γ secretion than group “HA2”, formulated iPR8 virus and alum.

Group “HA2” (iPR8+alum), “HA3” and “HA4” (iPR8 virus+RNA) showed relatively high IFN-γ secreted T cell population in case of stimulating with MHC I peptide. Besides, groups “HA9” and “HA10”, each of which formulated with RNA and LNP, was somewhat high IFN-γ secreted T cell population compared to groups “HA5” and “HA6”, formulated with only RNA, or groups “HA7” and “HA8” formulated with RNA and protamine.

### Experimental Example 10: Immunoassay of Nucleic Acid Molecule

Immunoassay using the nucleic acid molecules synthesized in Example 21 was performed to confirm the molecule of RNA platform as an adjuvant as Experimental Example 6. C57BL/6 mice aged 6 weeks were inoculated by intramuscular injection one or two times at 2 weeks interval with the following formulations; 1 μg/mice of MERS spite (S) soluble protein vaccine formulated with or without adjuvant, 20 μg/mice of RNA (pCrPV-MERS) pre-formulated with 10 μg of protamine and/or 500 μg/mice of alum (Thermo Scientific) as indicated in Table 7 below.

TABLE 7
Formulation of RNA and MERS Protein

| Group | G1 | G2 | G3 | G4 | G5 |
|---|---|---|---|---|---|
| MERS S protein | − | + | + | + | + |
| Alum | − | − | + | − | + |
| RNA (pCrPV-MERS) | − | − | − | + | + |

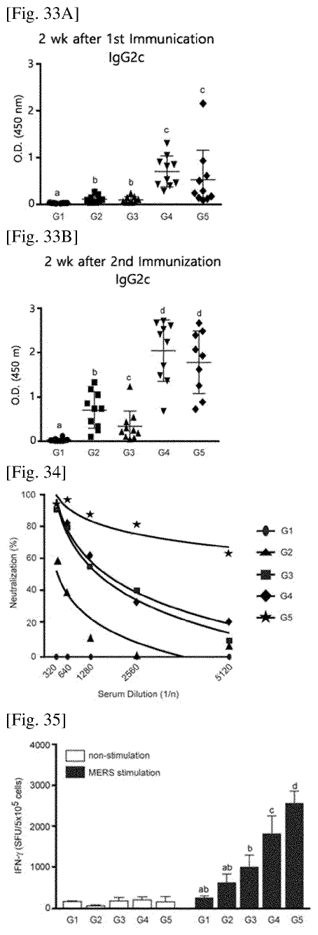

We performed immunoassays using sera of mice immunized with MERS S soluble protein vaccines with or without RNA (pCrPV-MERS). As illustrated in FIGS. 32A and 32B, at 2 weeks after 1st immunization, G5 (S protein+alum+RNA adjuvant) showed the highest IgG1 level (indicating a predominantly Th2 response), and G4 (S protein+RNA adjuvant) and G3 (S protein+alum) showed an increase in IgG1 compared to G2 (S protein). However, after boosting (2 weeks after 2nd immunization), G2 to G5 showed similar IgG1 levels. On the other hand, as illustrated in FIGS. 33A and 33B, IgG2c level (indicating a predominantly Th1 response) was only induced in G4 and G5, suggesting that the RNA (pCrPV-MERS) can induce Th1 response.

Moreover, G5 showed the highest neutralizing antibody levels (indicating strong Th2 responses) and G3 and G4 showed similar levels, as illustrated in FIG. 34. In addition, G5 and G4 showed higher IFN-γ secreting cells after stimulating with MERS S protein than G2 and G3 (See, FIG. 35), indicating that the RNA induced MERS S protein-specific Th1 responses.

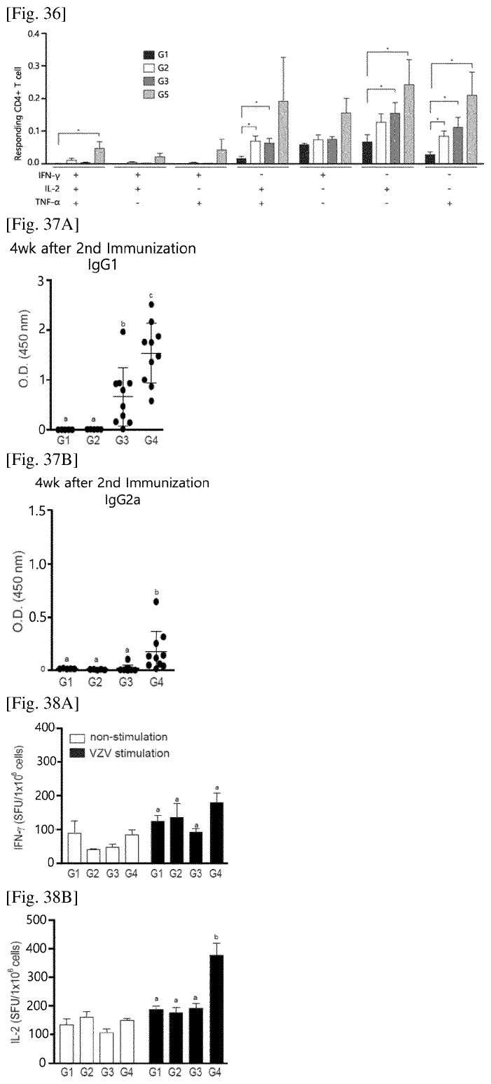

To perform flow cytometry assay, spleenocytes and isolated immune cells from muscle were stained with the CD4 (Clone GK1.5, 862 eBioscience; Clone H129.19, Bio Legend) for the surface staining. The stained cells were permeabilized using Cytofix/Cytoperm kit (eBioscience) and then stained with anti-IFN-γ-APC, anti-TNF-α-FITC, and anti-IL-2-PE (Clone XMG1.2, BD Biosciences; Clone MP6-XT22, Invitrogen; Clone JES6-5H4, eBioscience). Cells were fixed with 1% paraformaldehyde, analyzed using an LSRII flow cytometer (BD Biosciences), and T cells positive for the various combinations of cytokines and degranulation were analyzed and quantified using a Boolean gating function in FlowJo (TreeStar). FIG. 36 illustrates the frequencies of IFN-γ, IL-2, and TNF-α-1319 producing polyfunctional CD4 T cells were assessed by flow cytometry. As illustrated in FIG. 36, polyfunctional CD4 T cells, secreting various 222 immune-related cytokines, such as IFN-γ, IL-2, and TNF-α, were also highly increased in G5. These results suggest that the RNA adjuvant promotes CD4 T cell responses, especially Th1, consequently inducing to antigen-specific cellular immune responses. Furthermore, administering alum with the RNA adjuvant generated a synergistic effect, leading to an increase in the neutralizing antibody levels by stimulating balanced Th1/Th2 responses.

### Example 27: Fabrication of Nucleic Acid Molecule of RNA Platform

An artificial nucleic acid molecule of RNA platform including a viral IRES element derived from CrPV was fabricated by repeating the same process as Example 5 except using a template DNA including nucleotides (SEQ ID NO: 25) encoding VZV gE subunit in place of Renilla luciferase as an ORF. The template DNA has the following ordered nucleotides:

5′-KpnI recognition site (GGTATC)—T7 promoter (SEQ ID NO: 14)—CrPV IGR IRES (SEQ ID NO: 6) as IRES element—start codons (CCT GCT)—VZV gE subunit as ORF (SEQ ID NO: 20)—SV40 late polyadenylation signal sequence (SEQ ID NO: 15)—NotI recognition site (GCGGCCGC)—3′. The nucleic acid molecule fabricated in this example will be referred as “pCrPV-ZVZ”

### Experimental Example 11: Immunoassay of Nucleic Acid Molecule

Immunoassay using the nucleic acid molecules synthesized in Example 27 was performed to confirm the molecule of RNA platform as an adjuvant as Experimental Example 6. First, we investigated whether the RNA adjuvant encoding VZV gE gene (pCrPV-ZVZ) would enhance the immune response by VZV gE as a subunit protein vaccine. We primed C57BL/6 mice with 2000 PFU of live-attenuated VZV bulk 343 (Oka/SK; SK Bioscience (termed LAV) for mimicking the immune system of VZV seropositive individuals. At 5 weeks after priming all groups were immunized with the following formulations; 10 μg VZV gE protein with or without 20 μg of RNA (pCrPV-ZVZ). Also, Guinea pigs aged 6 week were primed with VZV bulk (Oka/SK) containing approximately 5000 780 PFU/mouse and inoculated by subcutaneous injection at 35 days after priming, two times at 2 week intervals with the following formulations; human dose (0.5 ml) of SkyZoster, which is a live-attenuated herpes zoster vaccine, with/without RNA adjuvant-VZV (50 μg). The formulation groups are indicted in Table 8 below.

TABLE 8
Formulation of RNA and ZVZ Protein

| Group | G1 | G2 | G3 | G4 |
|---|---|---|---|---|
| LAV priming | − | + | + | + |
| VZV gE protein | − | − | + | − |
| pCrPV-VZV | − | − | − | + |

IgG1 and IgG2a levels were analyzed with serum collected at 4 weeks after 2nd immunization from the primed mice. IgG1 and IgG2a levels of G4 were higher than G3, as illustrated in FIGS. 37A and 37B. In addition, VZV-specific cytokines (IFN-γ and IL-2), which are known Th1 cytokines released from the spleenocytes, were measured by ELISPOT to confirm VZV-specific Th1 response. The frequency of both IL-2 and IFN-γ secreting cells in the cultured spleenocytes increased about 2- to 3-fold in G4 compared to that in G3, as illustrated in FIGS. 38A and 38B. In particular, increase in the IL-2 secreting immune cells indicated the increases in T cell activation, expansion, development, and maintenance and the differentiation of CD8 T cells into terminal effector cells and memory cells. These results suggested that the RNA adjuvant-VZV can be an ideal adjuvant for VZV to increase the gE subunit vaccine efficacy by inducing T cell activation and cellular immune response.

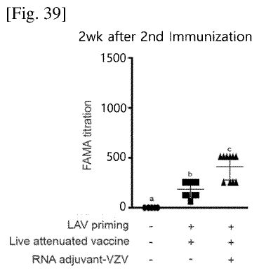

Next, we tested the effect of RNA adjuvant-VZV in the live-attenuated vaccine, SkyZoster (SK Bioscience), for routine shingles vaccination. After a 5 week priming (LAV) with 5000 PFU of VZV bulk (Oka/SK), the guinea pigs (GP) (Dunkin-Hartley strain, KOSA Bio) were immunized with various combinations of human dose (0.5 ml) of live-attenuated vaccine and 50 μg RNA (pCrPV-VZV) subcutaneously. Two doses at a 2 weeks interval were administered. VZV-specific neutralizing antibody, which was measured using fluorescent antibody to membrane antigen (FAMA) in the RAN. To determine anti-VZV IgG, 30 μL DPBS was added into U-bottom 96-well plates. Serum from the guinea pigs was serially diluted from 1/2 to 1/1024. Cell-associated virus (30 μL) from infected cells was added to wells and incubated for 30 min at 37° C. After centrifugation at 886 32 2000 rpm for 5 m, the supernatant was removed, and the cells were washed with 1% gelatin-DPBS (2:1) buffer and 30 μL 1/200 dilution of anti-human IgG-FITC conjugate was added to all wells and the plate was incubated for 30 m at 37° C. After washing with 1% gelatin-DPBS buffer, 4 μl glycerol-DPBS (2:1) was added to each well and visualized by confocal microscopy. As illustrated in FIG. 39, VZV-specific neutralizing antibody level in the RNA-adjuvant-VZV was about 2-fold higher than in the live-attenuated vaccine. Therefore, RNA (pCrPV-ZVZ) could activate the humoral immune response in live-attenuated vaccine similar to the protein-subunit vaccine.

While the present disclosure has been described with reference to exemplary embodiments and examples, these embodiments and examples are not intended to limit the scope of the present disclosure. Rather, it will be apparent to those skilled in the art that various modifications and variations can be made in the present disclosure without departing from the spirit or scope of the invention. Thus, it is intended that the present invention cover the modifications and variations of the present disclosure provided they come within the scope of the appended claims and their equivalents.

### INDUSTRIAL APPLICABILITY

The present disclosure relates to a nucleic acid molecule, and more specifically, to a nucleic acid molecule enhancing expression efficiency, a expression vector comprising the nucleic acid molecule and pharmaceutical use thereof.

1. A nucleic acid molecule comprising:
at least one expression control sequence comprising a viral Internal Ribosomal Entry Site (IRES) element having a viral 5′ untranslated region (5′ UTR);
at least one coding region linked operatively to the at least one expression control sequence and encoding a peptide or a protein; and
at least one of multiple adenosines and multiple thymidines located upstream of the at least one expression control sequence.

2. The nucleic acid molecule of claim 1, wherein the viral IRES element is derived from at least one of Picornaviridae family, Togaviridae family, Dicistroviridae family, Flaviridae family, Retroviridae family and Herpesviridae family.

3. The nucleic acid molecule of claim 1, wherein the viral IRES element is derived from at least one of coxsackie B virus, Cricket paralysis virus, Japanese Encephalitis virus, Encephalomyocarditis virus and Sindbis virus.

4. The nucleic acid molecule of claim 1, further comprising a viral 3′ untranslated region (3′ UTR) located downstream of the 5′ UTR, and wherein the at least one coding region is located between the 5′ UTR and the 3′ UTR.

5. The nucleic acid molecule of claim 1, wherein the at least one coding region encodes at least one of antigen, antigen's fragments, antigen's variants, antigen's derivatives, peptides for treating disease and proteins for treating disease.

6. The nucleic acid molecule of claim 1, wherein the at least one expression control sequence comprises a first expression control sequence having a first IRES element and a second expression control sequence located downstream of the first expression control sequence and having a second IRES element.

7. The nucleic acid molecule of claim 6, wherein the at least one coding region comprises a first coding region located between the first and second expression control sequences and a second coding region located downstream of the second expression control sequence.

8. The nucleic acid molecule of claim 6, wherein the first expression control sequence comprises a first viral IRES element derived from coxsackie B virus or Cricket paralysis virus, and the second expression control sequence comprises a second viral IRES element derived from Encephalomyocarditis virus.

9. The nucleic acid molecule of claim 1, further comprising a transcription control sequence upstream of the at least one expression control sequence, and a polyadenylation signal sequence or a poly adenosine located sequence downstream of the at least one coding region.

10. A recombinant vector comprising a nucleic acid molecule according to claim 1.

11. The recombinant vector of claim 10, wherein the at least one expression control sequence comprises a first expression control sequence having a first IRES element and a second expression control sequence located downstream of the first expression control sequence and having a second IRES element.

12. A method of stimulating an immune response in a subject, the method comprising administering a pharmaceutically effective amount of a nucleic acid molecule, wherein the nucleic acid molecule comprising: at least one expression control sequence comprising a viral Internal Ribosomal Entry Site (IRES) element having a viral 5′ untranslated region (5′ UTR).

13. The method of claim 12, wherein the nucleic acid molecule further comprise at least one coding region linked operatively to the at least one expression control sequence and encoding a peptide or a protein.

14. The method of claim 13, wherein the at least one coding region encodes an antigen or fragments thereof.

15. The method of claim 13, wherein the at least one coding region encodes a peptide or a protein selected from the group consisting of a viral pathogen, a viral antigen and combination thereof.

16. The method of claim 13, wherein the at least one expression control sequence comprises a first expression control sequence having a first IRES element and a second expression control sequence located downstream of the first expression control sequence and having a second IRES element.

17. The method of claim 16, wherein the at least one coding region comprises a first coding region located between the first and second expression control sequences and a second coding region located downstream of the second expression control sequence.

18. The method of claim 13, wherein the first expression control sequence comprises a first viral IRES element derived from coxsackie B virus or Cricket paralysis virus, and the second expression control sequence comprises a second viral IRES element derived from Encephalomyocarditis virus.

19. The method of claim 13, wherein the viral IRES element is derived from at least one of Picornaviridae family, Togaviridae family, Dicistroviridae family, Flaviridae family, Retroviridae family and Herpesviridae family.

20. The method of claim 16, wherein the viral IRES element is derived from at least one of coxsackie B virus, Cricket paralysis virus, Japanese Encephalitis virus, Encephalomyocarditis virus and Sindbis virus.

21. The method of claim 13, the nucleic acid molecule further comprises a viral 3′ untranslated region (3′ UTR) located downstream of the 5′ UTR, and wherein the at least one coding region is located between the 5′ UTR and the 3′ UTR.


SEQUENCE LISTING (40 sequences)

SEQ ID NO: 1 - DNA, 59 bp, Sindbis virus
attgacggcgtagtacacactattgaatcaaacagccgaccaattgcactaccatcaca

SEQ ID NO: 2 - DNA, 742 bp, Coxsackievirus B
ttaaaacagcctgtgggttgatcccacccacagggcctattgggcgctagcactctggtatcacggtacctttgtgcgcctgttttatatcccctcccccaactgtaacttagaagtaacacactccgatcaacagtcagcgtggcacaccagccatgttttgatcaagcacttctgttaccccggactgagtatcaatagactgctcacgcggttgaaggagaaagcgttcgttatccggccaactacttcgaaaaacccagtaacaccatagaggttgcagagtgtttcgctcagcactaccccagtgtagaccaggccgatgagtcaccgcattccccacgggcgaccgtggcggtggctgcgttggcggcctgcctatggggaaacccataggacgctctaatacagacatggtgcgaagagtctattgagctagttggtaatcctccggcccctgaatgcggctaatcctaactgcggacagcacaccctcaaaccagagggcagtgtgtcgtaacgggcaactctgcagcggaaccgactactttgggtgtccgtgtttcattttattcctatactggctgcttatggtgacaattgagagattgttaccatatagctattggattggccatccggtgtctaatagagctattatatatctctttgttggatttataccacttagcttgagagaggttaaaacattacaattcattgttaaattgaatacaacaaa

SEQ ID NO: 3 - DNA, 833 bp, Encephalomyocarditis virus
ttgaaagccgggggtgggagatccggattgccagtctgctcgatatcgcaggctgggtccgtgactacccactccccctttcaacgtgaaggctacgatagtgccagggcgggtactgccgtaagtgccaccccaaaataacaacagaccccccccccccccccccccccccccccccccccccccccccccccccccccccccccccccccccccccccccccccccccccccccccccccccccccccccccccccccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataat

SEQ ID NO: 4 - DNA, 574 bp, Encephalomyocarditis virus
cccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataat

SEQ ID NO: 5 - DNA, 95 bp, Japanese encephalitis virus
agaagtttatctgtgtgaacttcttggcttagtatcgttgagaagaatcgagagattagtgcagtttaaacagttttttagaacggaagataacc

SEQ ID NO: 6 - DNA, 189 bp, Cricket paralysis virus
aaagcaaaaatgtgatcttgcttgtaaatacaattttgagaggttaataaattacaagtagtgctatttttgtatttaggttagctatttagctttacgttccaggatgcctagtggcagccccacaatatccaggaagccctctctgcggtttttcagattaggtagtcgaaaaacctaagaaattta

SEQ ID NO: 7 - DNA, 319 bp, Sindbis virus
ccgctacgccccaatgatccgaccagcaaaactcgatgtacttccgaggaactgatgtgcataatgcatcaggctggtacattagatccccgcttaccgcgggcaatatagcaacactaaaaactcgatgtacttccgaggaagcgcagtgcataatgctgcgcagtgttgccacataaccactatattaaccatttatctagcggacgccaaaaactcaatgtatttctgaggaagcgtggtgcataatgccacgcagcgtctgcataacttttattatttcttttattaatcaacaaaattttgtttttaacatttc

SEQ ID NO: 8 - DNA, 103 bp, Coxsackievirus B
taaattagagacaatttgatctgatttgaattggcttaaccctactgtactaaccgaactagacaacggtgcagtaggggtaaattctccgcattcggtgcgg

SEQ ID NO: 9 - DNA, 123 bp, Encephalomyocarditis virus
tagtgtagtcactggcacaacgcgttacccggtaagccaatcgggtatacacggtcgtcatactgcagacagggttcttctactttgcaagatagtctagagtagtaaaataaatagatagag

SEQ ID NO: 10 - DNA, 585 bp, Japanese encephalitis virus
tagtgtgatttaaagtagaaaagtagactatgtaaataatgtaaatgagaaaatgcatgcatatggagtcaggccagcaaaagctgccaccggatactgggtagacggtgctgtctgcgtctcagtcccaggaggactgggttaacaaatctgacaacagaaagtgagaaagccctcagaaccgtctcggaagtaggtccctgctcactggaagttgaaagaccaacgtcaggccacaaatttgtgccaccccgctagggggtgcggcctgcgcagccccaggaggactgggttaccaaagccgttgagcccccacggcccaagcctcgtctaggatgcaatagacgaggtgtaaggactagaggttagaggagaccccgtggaaacaacaatatgcggcccaagccccctcgaagctgtagaggaggtggaaggactagaggttagaggagaccccgcatttgcatcaaacagcatattgacacctgggaatagactgggagatcttctgctctatctcaacatcagctactaggcacagagcgccgaagtatgtagctggtggtgaggaagaacacaggatct

SEQ ID NO: 11 - DNA, 144 bp, Sindbis virus
atggagaagccagtagtaaacgtagacgtagacccccagagtccgtttgtcgtgcaactgcaaaaaagcttcccgcaatttgaggtagtagcacagcaggtcactccaaatgaccatgctaatgccagagcattttcgcatctg

SEQ ID NO: 12 - DNA, 12 bp, Artificial Sequence
  Expression Enhancer Sequence
atggcagctcaa

SEQ ID NO: 13 - DNA, 102 bp, Japanese encephalitis virus
atgactaaaaaaccaggagggcccggtaaaaaccgggctatcaatatgctgaaacgcggcctaccccgcgtattcccactagtgggagtgaagagggtagta

SEQ ID NO: 14 - DNA, 18 bp, Bacteriophage T7
taatacgactcactatag

SEQ ID NO: 15 - DNA, 220 bp, Simian virus 40
agacatgataagatacattgatgagtttggacaaaccacaacaagaatgcagtgaaaaaaatgctttatttgtgaaatttgtgatgctattgctttatttgtaaccattataagctgcaataaacaagttaacaacaacaatgcattcattttatgtttcaggttcagggggagatgtgggaggttttttaaagcaagtaaaacctctacaaatgtggta

SEQ ID NO: 16 - DNA, 936 bp, Renilla reniformis
atgacttcgaaagtttatgatccagaacaaaggaaacggatgataactggtccgcagtggtgggccagatgtaaacaaatgaatgttcttgattcatttattaattattatgattcagaaaaacatgcagaaaatgctgttatttttttacatggtaacgcggcctcttcttatttatggcgacatgttgtgccacatattgagccagtagcgcggtgtattataccagaccttattggtatgggcaaatcaggcaaatctggtaatggttcttataggttacttgatcattacaaatatcttactgcatggtttgaacttcttaatttaccaaagaagatcatttttgtcggccatgattggggtgcttgtttggcatttcattatagccatgagcatcaagataagatcaaagcaatagttcacgctgaaagtgtagtagatgtgattgaatcatgggatgaatggcctgatattgaagaagatattgcgttgatcaaatctgaagaaggagaaaaaatggttttggagaataacttcttcgtggaaaccatgttgccatcaaaaatcatgagaaagttagaaccagaagaatttgcagcatatcttgaaccattcaaagagaaaggtgaagttcgtcgtccaacattatcatggcctcgtgaaatcccgttagtaaaaggtggtaaacctgacgttgtacaaattgttaggaattataatgcttatctacgtgcaagtgatgatttaccaaaaatgtttattgaatcggacccaggattcttttccaatgctattgttgaaggtgccaagaagtttcctaatactgaatttgtcaaagtaaaaggtcttcatttttcgcaagaagatgcacctgatgaaatgggaaaatatatcaaatcgttcgttgagcgagttctcaaaaatgaacaataa

SEQ ID NO: 17 - DNA, 933 bp, Renilla reniformis
acttcgaaagtttatgatccagaacaaaggaaacggatgataactggtccgcagtggtgggccagatgtaaacaaatgaatgttcttgattcatttattaattattatgattcagaaaaacatgcagaaaatgctgttatttttttacatggtaacgcggcctcttcttatttatggcgacatgttgtgccacatattgagccagtagcgcggtgtattataccagaccttattggtatgggcaaatcaggcaaatctggtaatggttcttataggttacttgatcattacaaatatcttactgcatggtttgaacttcttaatttaccaaagaagatcatttttgtcggccatgattggggtgcttgtttggcatttcattatagctatgagcatcaagataagatcaaagcaatagttcacgctgaaagtgtagtagatgtgattgaatcatgggatgaatggcctgatattgaagaagatattgcgttgatcaaatctgaagaaggagaaaaaatggttttggagaataacttcttcgtggaaaccatgttgccatcaaaaatcatgagaaagttagaaccagaagaatttgcagcatatcttgaaccattcaaagagaaaggtgaagttcgtcgtccaacattatcatggcctcgtgaaatcccgttagtaaaaggtggtaaacctgacgttgtacaaattgttaggaattataatgcttatctacgtgcaagtgatgatttaccaaaaatgtttattgaatcggacccaggattcttttccaatgctattgttgaaggtgccaagaagtttcctaatactgaatttgtcaaagtaaaaggtcttcatttttcgcaagaagatgcacctgatgaaatgggaaaatatatcaaatcgttcgttgagcgagttctcaaaaatgaacaataa

SEQ ID NO: 18 - DNA, 1653 bp, Unknown
  Firefly
atggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaa

SEQ ID NO: 19 - DNA, 4017 bp, Unknown
  Middle East respiratory syndrome coronavirus (MERS-CoV)
atgattcactctgtgttcctgctgatgttcctgctgacaccaacagagtcctatgtggatgtgggacctgactctgtgaagtctgcctgtattgaggtggacatccaacagaccttctttgacaagacctggccaagaccaattgatgtgagcaaggctgatggcatcatctacccacagggcaggacctacagcaacatcaccatcacctaccagggactgtttccataccagggagatcatggagatatgtatgtctactctgctggtcatgccacaggcaccacaccacagaaactgtttgtggctaactacagccaggatgtgaagcagtttgccaatggctttgtggtgaggattggagcagcagccaacagcacaggcacagtgattatcagcccaagcacctctgccaccatcaggaagatttaccctgcctttatgctgggctcctctgtgggcaacttctctgatggcaagatgggcaggttcttcaaccacaccctggtgctgctgcctgatggctgtggcaccctgctgagggctttctactgtatcttggaaccaaggtctggcaaccactgtcctgctggcaactcctacacctcctttgccacctaccacacacctgccacagactgttctgatggcaactacaacaggaatgcctccctgaactccttcaaggaatacttcaacctgaggaactgtacctttatgtacacctacaacatcacagaggatgagattttggagtggtttggcatcacccagacagcccagggagtgcatctgttctcgagcagatatgtggacctctatggaggcaatatgttccagtttgccaccctgcctgtctatgacaccatcaaatactacagcatcatcccacacagcatcaggagcatccagtctgacaggaaggcttgggctgccttctatgtctacaaactccaaccactgaccttcctgctggacttctctgtggatggctacatcaggagggctattgactgtggcttcaatgacctgagccaacttcactgttcctatgagtcctttgatgtggagtctggagtctactctgtgtcctcctttgaggctaagccatctggctctgtggtggaacaggctgagggagtggagtgtgacttcagcccactgctgtctggcacacctccacaggtctacaacttcaagagactggtgttcaccaactgtaactacaacctgaccaaactgctgtccctgttctctgtgaatgacttcacttgtagccagattagccctgctgccattgccagcaactgttactcctccctgattctggactacttctcctacccactgagtatgaagtctgacctgtctgtgtcctctgctggaccaatcagccagttcaactacaagcagtccttcagcaacccaacttgtctgattctggctacagtgccacacaacctgaccaccatcaccaagccactgaaatactcctacatcaacaagtgtagcagactgctgtctgatgacaggacagaggtgccacaactagtgaatgccaaccaatacagcccatgtgtgagcattgtgccaagcacagtgtgggaggatggagactactacaggaagcaacttagcccattggagggaggaggctggctggtggcatctggcagcacagtggctatgacagaacaactccaaatgggctttggcatcacagtccaatatggcacagacaccaactctgtgtgtccaaaattggagtttgccaatgacaccaagattgccagccaacttggcaactgtgtggaatactccctctatggagtgtctggcaggggagtgttccagaactgtactgctgtgggagtgagacaacagaggtttgtctatgatgcctaccagaacctggtgggctactactctgatgatggcaactactactgtctgagggcttgtgtgtctgtgcctgtgtctgtgatttatgacaaggagaccaagacccatgccaccctgtttggctctgtggcttgtgaacacatctccagcacaatgagtcaatacagcaggagcaccaggagtatgctgaaaaggagggacagcacatatggaccactccaaacacctgtgggctgtgtgctgggactggtgaactcctccctgtttgtggaggactgtaaactgccactgggacaatccctgtgtgccctgcctgacacaccaagcaccctgacaccaaggtctgtgaggtctgtgcctggagagatgagactggcaagcattgccttcaaccacccaatccaggtggaccaacttaactcctcctacttcaaactgagcatcccaaccaacttctcctttggagtgacccaggaatacatccagaccaccatccagaaggtgacagtggactgtaagcaatatgtgtgtaatggcttccagaagtgtgaacaacttctgagggaatatggacaattctgtagcaagataaaccaggctcttcatggagccaacctgagacaggatgactctgtgaggaacctgtttgcctctgtgaagtccagccagtccagcccaatcatccctggctttggaggagacttcaacctgaccctgttggaaccggtgagcatcagcacaggcagcaggtctgccaggtctgccattgaggacctgctgtttgacaaggtgaccattgctgaccctggctatatgcagggctatgatgactgtatgcaacagggacctgcctctgccagggacctgatttgtgcccaatatgtggctggctacaaggtgctgcctccactgatggatgtgaatatggaggctgcctacacctcctccctgctgggcagcattgctggagtgggctggactgcaggactgtcctcctttgctgccatcccatttgcccagagcatcttctacagactgaatggagtgggcatcacccaacaggtgctgtctgagaaccagaaactgattgccaacaagttcaaccaggctctgggagctatgcagacaggcttcaccaccaccaatgaggctttccagaaggtccaggatgctgtgaacaacaatgcccaggctctgagcaaactggcatctgaactgagcaacacctttggagccatctctgctagcattggagacatcatccagagactggatgtgttggaacaggatgcccagattgacagactgataaatggcagactgaccaccctgaatgcctttgtggctcaacaacttgtgaggtctgagtctgctgccctgtctgcccaacttgccaaggacaaggtgaatgagtgtgtgaaggctcaaagcaagaggtctggcttctgtggacaaggcacccacattgtgtcctttgtggtgaatgccccaaatggactctactttatgcatgtgggctactacccaagcaaccacattgaggtggtgtctgcctatggactgtgtgatgctgccaacccaaccaactgtattgcccctgtgaatggctacttcatcaagaccaacaacaccaggattgtggatgagtggtcctacacaggctcctccttctatgcccctgaaccaatcacctccctgaacaccaaatatgtggctccacaggtgacctaccagaacatcagcaccaacctgcctcctccactgctgggcaacagcacaggcattgacttccaggatgaactggatgagttcttcaagaatgtgagcaccagcatcccaaactttggctccctgacccagataaacaccaccctgctggacctgacctatgagatgctgtccctccaacaggtggtgaaggctctgaatgagtcctacattgacctgaaagaactgggcaactacacctactacaacaagtggccatggtacatctggctgggcttcatcgctggcctggtggccctggcgctgtgcgtgttcttcatcctgtgctgcaccggctgcggcaccaactgcatgggcaagctgaagtgcaacaggtgctgcgactaa

SEQ ID NO: 20 - protein, 1296 aa, Unknown
  Middle East respiratory syndrome coronavirus (MERS-CoV)
MIHSVFLLMFLLTPTESYVDVGPDSVKSACIEVDIQQTFFDKTWPRPIDVSKADGIIYPQGRTYSNITITYQGLFPYQGDHGDMYVYSAGHATGTTPQKLFVANYSQDVKQFANGFVVRIGAAANSTGTVIISPSTSATIRKIYPAFMLGSSVGNFSDGKMGRFFNHTLVLLPDGCGTLLRAFYCILEPRSGNHCPAGNSYTSFATYHTPATDCSDGNYNRNASLNSFKEYFNLRNCTFMYTYNITEDEILEWFGITQTAQGVHLFSSRYVDLYGGNMFQFATLPVYDTIKYYSIIPHSIRSIQSDRKAWAAFYVYKLQPLTFLLDFSVDGYIRRAIDCGFNDLSQLHCSYESFDVESGVYSVSSFEAKPSGSVVEQAEGVECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRLLSDDRTEVPQLVNANQYSPCVSIVPSTVWEDGDYYRKQLSPLEGGGWLVASGSTVAMTEQLQMGFGITVQYGTDTNSVCPKLEFANDTKIASQLGNCVEYSLYGVSGRGVFQNCTAVGVRQQRFVYDAYQNLVGYYSDDGNYYCLRACVSVPVSVIYDKETKTHATLFGSVACEHISSTMSQYSRSTRSMLKRRDSTYGPLQTPVGCVLGLVNSSLFVEDCKLPLGQSLCALPDTPSTLTPRSVRSVPGEMRLASIAFNHPIQVDQLNSSYFKLSIPTNFSFGVTQEYIQTTIQKVTVDCKQYVCNGFQKCEQLLREYGQFCSKINQALHGANLRQDDSVRNLFASVKSSQSSPIIPGFGGDFNLTLLEPVSISTGSRSARSAIEDLLFDKVTIADPGYMQGYDDCMQQGPASARDLICAQYVAGYKVLPPLMDVNMEAAYTSSLLGSIAGVGWTAGLSSFAAIPFAQSIFYRLNGVGITQQVLSENQKLIANKFNQALGAMQTGFTTTNEAFQKVQDAVNNNAQALSKLASELSNTFGAISASIGDIIQRLDVLEQDAQIDRLINGRLTTLNAFVAQQLVRSESAALSAQLAKDKVNECVKAQSKRSGFCGQGTHIVSFVVNAPNGLYFMHVGYYPSNHIEVVSAYGLCDAANPTNCIAPVNGYFIKTNNTRIVDEWSYTGSSFYAPEPITSLNTKYVAPQVTYQNISTNLPPPLLGNSTGIDFQDELDEFFKNVSTSIPNFGSLTQINTTLLDLTYEMLSLQQVVKALNESYIDLKELGNYTYYNKWP

SEQ ID NO: 21 - DNA, 1570 bp, Human papillomavirus type 16
atgagcctgtggctgcccagcgaggccaccgtgtacctgccccccgtgcccgtgagcaaggtggtgagcaccgacgagtacgtggccaggaccaacatctactaccacgccggcaccagcaggctgctggccgtgggccacccctacttccccatcaagaagcccaacaacaacaagatcctggtgcccaaggtgagcggcctgcagtacagggtgttcaggatccacctgcccgaccccaacaagttcggcttccccgacaccagcttctacaaccccgacacccagaggctggtgtgggcctgcgtgggcgtggaggtgggcaggggccagcccctgggcgtgggcatcagcggccaccccctgctgaacaagctggacgacaccgagaacgccagcgcctacgccgccaacgccggcgtggacaacagggagtgcatcagcatggactacaagcagacccagctgtgcctgatcggctgcaagccccccatcggcgagcactggggcaagggcagcccctgcaccaacgtggccgtgaaccccggcgactgcccccccctggagctgatcaacaccgtgatccaggacggcgacatggtggacaccggcttcggcgccatggacttcaccaccctgcaggccaacaagagcgaggtgcccctggacatctgcaccagcatctgcaagtaccccgactacatcaagatggtgagcgagccctacggcgacagcctgttcttctacctgaggagggagcagatgttcgtgaggcacctgttcaacagggccggcgccgtgggcgagaacgtgcccgacgacctgtacatcaagggcagcggcagcaccgccaacctggccagcagcaactacttccccacccccagcggcagcatggtgaccagcgacgcccagatcttcaacaagccctactggctgcagagggcccagggccacaacaacggcatctgctggggcaaccagctgttcgtgaccgtggtggacaccaccaggagcaccaacatgagcctgtgcgccgccatcagcaccagcgagaccacctacaagaacaccaacttcaaggagtacctgaggcacggcgaggagtacgacctgcagttcatcttccagctgtgcaagatcaccctgaccgccgacgtgatgacctacatccacagcatgaacagcaccatcctggaggactggaacttcggcctgcagcccccccccggcggcaccctggaggacacctacaggttcgtgaccagccaggccatcgcctgccagaagcacaccccccccgcccccaaggaggaccccctgaagaagtacaccttctgggaggtgaacctgaaggagaagttcagcgccgacctggaccagttccccctgggcaggaagttcctgctgcaggccggcctgaaggccaagcccaagttcaccctgggcaagaggaaggccacccccaccaccagcagcaccagcaccaccgccaagaggaagaagaggaagctgtgaaagctacccacggccgaatagccgtgagccggaatcctgcacgctagcatta

SEQ ID NO: 22 - DNA, 1524 bp, Human papillomavirus type 18
atggccctctggagaccatccgataacacagtgtacttgcccccacccagcgtcgcccgggtggtgaacacagacgactacgtcaccagaacctcaatcttctaccacgccgggtccagccggctgctgaccgtgggcaacccctacttccgcgtgcccgccggcggcggaaacaaacaagacatccccaaagtcagcgcctatcagtaccgggtgttccgcgtccaactgcccgatcccaacaagttcggcctgcccgacacctccatctacaaccccgagacccagaggctggtctgggcttgcgccggcgtcgagatcgggaggggccaacccctgggcgtggggttgtccggccaccccttctacaacaagctggacgataccgagtccagccacgcagcaaccagcaacgtctccgaagatgtgcgcgataacgtcagcgtggactacaaacaaacccaactgtgcatcctgggatgcgcacccgccatcggcgagcattgggccaaggggaccgcctgcaagagcaggcccctgagccaaggggactgtccacccctggagttgaagaataccgtgctcgaggacggcgacatggtggacaccggctacggcgctatggatttctccaccctccaggacaccaagtgcgaagtgcccctcgacatctgccaaagcatctgcaagtaccccgactacctccagatgagcgccgacccctacggcgacagcatgttcttctgtctcagaagggaacaattgttcgcccgccacttctggaaccgggccggcacaatgggagatacagtcccccagagcctgtacatcaaggggaccggaatgagggccagccccgggtcctgcgtctacagcccaagcccctccgggagcatcgtcacaagcgatagccaactcttcaacaagccctactggctccacaaagcccaaggccacaataacggggtgtgttggcacaaccagctgttcgtgaccgtcgtggacacaaccaggtccacaaacctgaccatctgcgccagcacccaaagccccgtgcccggccagtacgacgccacaaagttcaaacaatactctcggcacgtggaagagtacgacctccaattcatcttccaactctgcaccatcaccctcaccgccgacgtgatgagctacatccactccatgaactcctccatcctggaagactggaatttcggcgtgccaccaccccctaccacctccctcgtcgacacctacagattcgtgcagagcgtggccatcacatgccagaaagacgccgcccccgccgagaacaaagacccatacgacaaactgaaattctggaacgtcgacctgaaagagaaattcagcctggatctggaccagtacccattgggcaggaagttcctcgtgcaagccggcctcaggagaaaaccaacaatcgggcccaggaagaggagcgcccccagcgcaaccaccagcagcaagcccgcaaaaagggtcagagtgagggcacgcaaatga

SEQ ID NO: 23 - DNA, 1698 bp, Influenza A virus
atgaaggccaacctgctcgtgctgctgtgcgccctcgcggccgccgacgccgacaccatctgcatcggctaccacgccaacaacagcaccgacacggtcgacaccgtgctggagaagaacgtgaccgtcacccactccgtgaacctgctcgaggacagccacaacgggaagctgtgccggctgaagggcatcgcgcccctccagctggggaagtgcaacatcgccggctggctgctcgggaacccggagtgcgaccccctgctgcccgtgcgctcctggagctacatcgtcgagacgcccaactccgagaacggcatctgctacccgggcgacttcatcgactacgaggaactccgggagcagctgagttccgtgagttccttcgagcgcttcgagatcttccccaaggagagttcctggcccaaccacaacaccaacggggtgaccgccgcctgcagccacgagggcaagtccagcttctaccggaacctgctctggctgaccgagaaggaggggtcctaccccaagctgaagaacagctacgtcaacaagaagggcaaggaggtgctcgtgctgtgggggatccaccacccgcccaactccaaggagcagcagaacctgtaccagaacgagaacgcgtacgtcagcgtggtgacgtccaactacaaccgccggttcacccccgagatcgccgagcgccccaaggtccgggaccaggccggccgcatgaactactactggaccctcctgaagccgggcgacaccatcatcttcgaggccaacgggaacctgatcgccccgatgtacgcgttcgccctcagccggggcttcgggagcggcatcatcacgtccaacgccagcatgcacgagtgcaacaccaagtgccagacccccctgggcgccatcaactccagcctgccctaccagaacatccacccggtgaccatcggggagtgccccaagtacgtgcgctccgccaagctccggatggtcacgggcctgcgcaacaaccccagcatccagtcccgggggctgttcggcgcgatcgccgggttcatcgagggcggctggaccgggatgatcgacggctggtacgggtaccaccaccagaacgagcagggcagcgggtacgccgccgaccagaagtccacccagaacgccatcaacggcatcaccaacaaggtgaacacggtgatcgagaagatgaacatccagttcaccgcggtcggcaaggagttcaacaagctcgagaagcgcatggagaacctgaacaagaaggtggacgacgggttcctggacatctggacctacaacgccgaactcctggtgctgctcgagaacgagcggaccctggacttccacgacagcaacgtcaagaacctgtacgagaaggtgaagtcccagctcaagaacaacgccaaggagatcggcaacgggtgcttcgagttctaccacaagtgcgacaacgagtgcatggagagcgtccgcaacggcacgtacgactaccccaagtactccgaggagagcaagctgaaccgggagaaggtggacggggtgaagctggagtccatgggcatctaccagatcctcgccatctacagcaccgtcgcctccagcctggtgctgctggtgtccctcggcgcgatcagcttctggatgtgcagcaacgggtccctgcagtgccgcatctgcatctga

SEQ ID NO: 24 - protein, 565 aa, Unknown
  Inactivated Influenza Vaccine
MKANLLVLLCALAAADADTICIGYHANNSTDTVDTVLEKNVTVTHSVNLLEDSHNGKLCRLKGIAPLQLGKCNIAGWLLGNPECDPLLPVRSWSYIVETPNSENGICYPGDFIDYEELREQLSSVSSFERFEIFPKESSWPNHNTNGVTAACSHEGKSSFYRNLLWLTEKEGSYPKLKNSYVNKKGKEVLVLWGIHHPPNSKEQQNLYQNENAYVSVVTSNYNRRFTPEIAERPKVRDQAGRMNYYWTLLKPGDTIIFEANGNLIAPMYAFALSRGFGSGIITSNASMHECNTKCQTPLGAINSSLPYQNIHPVTIGECPKYVRSAKLRMVTGLRNIPSIQSRGLFGAIAGFIEGGWTGMIDGWYGYHHQNEQGSGYAADQKSTQNAINGITNKVNTVIEKMNIQFTAVGKEFNKLEKRMENLNKKVDDGFLDIWTYNAELLVLLENERTLDFHDSNVKNLYEKVKSQLKNNAKEIGNGCFEFYHKCDNECMESVRNGTYDYPKYSEESKLNREKVDGVKLESMGIYQILAIYSTVASSLVLLVSLGAISFWMCSNGSLQCRICI

SEQ ID NO: 25 - DNA, 1866 bp, Unknown
  Varicella-zoster virus (VZV)
gggacagttaataaacctgtggtgggggtattgatggggttcggaattatcacgggaacgttgcgtataacgaatccggtcagagcatccgtcttgcgatacgatgattttcacatcgatgaagacaaactggatacaaactccgtatatgagccttactaccattcagatcatgcggagtcttcatgggtaaatcggggagagtcttcgcgaaaagcgtacgatcataactcaccttatatatggccacgtaatgattatgatggatttttagagaacgcacacgaacaccatggggtgtataatcagggccgtggtatcgatagcggggaacggttaatgcaacccacacaaatgtctgcacaggaggatcttggggacgatacgggcatccacgttatccctacgttaaacggcgatgacagacataaaattgtaaatgtggaccaacgtcaatacggtgacgtgtttaaaggagatcttaatccaaaaccccaaggccaaagactcattgaggtgtcagtggaagaaaatcacccgtttactttacgcgcaccgattcagcggatttatggagtccggtacaccgagacttggagctttttgccgtcattaacctgtacgggagacgcagcgcccgccatccagcatatatgtttaaaacatacaacatgctttcaagacgtggtggtggatgtggattgcgcggaaaatactaaagaggatcagttggccgaaatcagttaccgttttcaaggtaagaaggaagcggaccaaccgtggattgttgtaaacacgagcacactgtttgatgaactcgaattagacccccccgagattgaaccgggtgtcttgaaagtacttcggacagaaaaacaatacttgggtgtgtacatttggaacatgcgcggctccgatggtacgtctacctacgccacgtttttggtcacctggaaaggggatgaaaaaacaagaaaccctacgcccgcagtaactcctcaaccaagaggggctgagtttcatatgtggaattaccactcgcatgtattttcagttggtgatacgtttagcttggcaatgcatcttcagtataagatacatgaagcgccatttgatttgctgttagagtggttgtatgtccccatcgatcctacatgtcaaccaatgcggttatattctacgtgtttgtatcatcccaacgcaccccaatgcctctctcatatgaattccggttgtacatttacctcgccacatttagcccagcgtgttgcaagcacagtgtatcaaaattgtgaacatgcagataactacaccgcatattgtctgggaatatctcatatggagcctagctttggtctaatcttacacgacgggggcaccacgttaaagtttgtagatacacccgagagtttgtcgggattatacgtttttgtggtgtattttaacgggcatgttgaagccgtagcatacactgttgtatccacagtagatcattttgtaaacgcaattgaagagcgtggatttccgccaacggccggtcagccaccggcgactactaaacccaaggaaattacccccgtaaaccccggaacgtcaccacttctacgatatgccgcatggaccggagggcttgcagcagtagtacttttatgtctcgtaatatttttaatctgtacggctaaacgaatgagggttaaagcctatagggtagacaagtccccgtataaccaaagcatgtattacgctggccttccagtggacgatttcgaggactcggaatctacggatacggaagaagagtttggtaacgcgattggagggagtcacgggggttcgagttacacggtgtatatagataagacccgg

SEQ ID NO: 26 - DNA, 44 bp, Homo sapiens
ggggcgctgcctacggaggtggcagccatctccttctcggcatc

SEQ ID NO: 27 - DNA, 70 bp, Homo sapiens
gtccacctgtccctcctgggctgctggattgtctcgttttcctgccaaataaacaggatcagcgctttac

SEQ ID NO: 28 - DNA, 28 bp, Unknown
  Histone stem-loop
aaaggctcttttcagagccaccagaatt

SEQ ID NO: 29 - DNA, 12 bp, Artificial Sequence
  Synthetic
atggcagctcaa

SEQ ID NO: 30 - DNA, 10 bp, Artificial Sequence
  Synthetic
ggatccgacc

SEQ ID NO: 31 - DNA, 12 bp, Artificial Sequence
  Synthetic
ccgcggcgatcg

SEQ ID NO: 32 - DNA, 10 bp, Artificial Sequence
  Synthetic
aagcttgagg

SEQ ID NO: 33 - DNA, 10 bp, Artificial Sequence
  Synthetic
gaattcgacc

SEQ ID NO: 34 - DNA, 24 bp, Artificial Sequence
  Synthetic
gatatcgtcgacttaattaagacc

SEQ ID NO: 35 - DNA, 26 bp, Artificial Sequence
  Synthetic
gaattcatcgatttaattaagagctc

SEQ ID NO: 36 - DNA, 26 bp, Artificial Sequence
  Synthetic
ggatccatcgatttaattaagagctc

SEQ ID NO: 37 - DNA, 26 bp, Artificial Sequence
  Synthetic
ggatccgaattcttaattaagagctc

SEQ ID NO: 38 - DNA, 24 bp, Artificial Sequence
  Synthetic
gtcgacgatatcccgcggcgatcg

SEQ ID NO: 39 - DNA, 24 bp, Artificial Sequence
  Synthetic
ggatccccgcgggtcgaccgatcg

SEQ ID NO: 40 - DNA, 18 bp, Artificial Sequence
  Synthetic
gatatcgcgagcgaattc